SEQUENCE LISTING (103 sequences)

SEQ ID NO: 1 - protein, 9 aa, Homo sapiens
  SITE (4)..(5): The peptide is optionally O-GlcNAcylated at one or both of these sites
ALTTSAHSV

SEQ ID NO: 2 - protein, 10 aa, Homo sapiens
  SITE (4)..(6): The peptide is optionally O-GlcNAcylated at one or more of these sites
  SITE (4)..(6): The peptide is optionally hexose-GlcNAcylated at one or more of these sites
  SITE (6)..(6): The peptide is optionally O-GlcNAcylated at this site
APPSTSAAAL

SEQ ID NO: 3 - protein, 9 aa, Homo sapiens
  SITE (5)..(5): The peptide is optionally hexose-GlcNAcylated at this site
APRGNVTSL

SEQ ID NO: 4 - protein, 9 aa, Homo sapiens
  SITE (4)..(4): The peptide is optionally O-GlcNAcylated at this site
APRTNGVAM

SEQ ID NO: 5 - protein, 8 aa, Homo sapiens
  SITE (4)..(4): The peptide is optionally O-GlcNAcylated at this site
APTSAAAL

SEQ ID NO: 6 - protein, 9 aa, Homo sapiens
  SITE (4)..(4): The peptide is optionally O-GlcNAcylated at this site
  SITE (6)..(6): The peptide is optionally O-GlcNAcylated at this site
APTSASNVM

SEQ ID NO: 7 - protein, 9 aa, Homo sapiens
  SITE (4)..(4): The peptide is optionally O-GlcNAcylated at this site
APVSASASV

SEQ ID NO: 8 - protein, 9 aa, Homo sapiens
  SITE (4)..(4): The peptide is optionally O-GlcNAcylated at this site
APVSSKSSL

SEQ ID NO: 9 - protein, 9 aa, Homo sapiens
  SITE (3)..(5): The peptide is optionally O-GlcNAcylated at one of these sites
EPSSTVVSL

SEQ ID NO: 10 - protein, 10 aa, Homo sapiens
  SITE (4)..(4): The peptide is optionally O-GlcNAcylated at this site
GLSSLAEEAA

SEQ ID NO: 11 - protein, 9 aa, Homo sapiens
  SITE (4)..(4): The peptide is optionally O-GlcNAcylated at this site
HPMSTASQV

SEQ ID NO: 12 - protein, 9 aa, Homo sapiens
  SITE (4)..(4): The peptide is optionally O-GlcNAcylated at one or both of these sites
HPSSTAAVL

SEQ ID NO: 13 - protein, 9 aa, Homo sapiens
  SITE (3)..(3): The peptide is optionally O-GlcNAcylated at these sites
  SITE (5)..(5): The peptide is optionally O-GlcNAcylated at these sites
HPSSTAAVL

SEQ ID NO: 14 - protein, 10 aa, Homo sapiens
  SITE (3)..(5): The peptide is optionally O-GlcNAcylated at one of these sites
HPSSTASTAL

SEQ ID NO: 15 - protein, 8 aa, Homo sapiens
  SITE (4)..(4): The peptide is optionally O-GlcNAcylated at this site
HPTTVASY

SEQ ID NO: 16 - protein, 9 aa, Homo sapiens
  SITE (4)..(4): The peptide is optionally O-GlcNAcylated at this site
IPISLHTSL

SEQ ID NO: 17 - protein, 9 aa, Homo sapiens
  SITE (4)..(4): The peptide is optionally O-GlcNAcylated at this site
IPTSSVLSL

SEQ ID NO: 18 - protein, 9 aa, Homo sapiens
  SITE (4)..(4): The peptide is optionally O-GlcNAcylated at this site
  SITE (4)..(4): The peptide is optionally hexose-GlcNAcylated at this site
IPVSKPLSL

SEQ ID NO: 19 - protein, 9 aa, Homo sapiens
  SITE (4)..(4): The peptide is optionally O-GlcNAcylated at this site
  SITE (4)..(5): The peptide is optionally O-GlcNAcylated at both of these sites
  SITE (4)..(4): The peptide is optionally hexose-GlcNAcylated at this site
IPVSSHNSL

SEQ ID NO: 20 - protein, 10 aa, Homo sapiens
  SITE (4)..(5): The peptide is optionally O-GlcNAcylated at one of these sites
  SITE (5)..(5): The peptide is optionally hexose-GlcNAcylated at this site
KPPTSQSSVL

SEQ ID NO: 21 - protein, 9 aa, Homo sapiens
  SITE (4)..(4): The peptide is optionally hexose-GlcNAcylated at this site
KPPVSFFSL

SEQ ID NO: 22 - protein, 9 aa, Homo sapiens
  SITE (5)..(6): The peptide is optionally hexose-GlcNAcylated at one of these sites
KPTLYNVSL

SEQ ID NO: 23 - protein, 8 aa, Homo sapiens
  SITE (5)..(6): The peptide is optionally O-GlcNAcylated at these sites
LPRNSTMM

SEQ ID NO: 24 - protein, 9 aa, Homo sapiens
  SITE (4)..(4): The peptide is optionally O-GlcNAcylated at this site
LPTSLPSSL

SEQ ID NO: 25 - protein, 10 aa, Homo sapiens
  SITE (7)..(7): The peptide is optionally O-GlcNAcylated at this site
MPVRPTTNTF

SEQ ID NO: 26 - protein, 9 aa, Homo sapiens
  SITE (4)..(4): The peptide is optionally O-GlcNAcylated at this site
MPVTSSSFF

SEQ ID NO: 27 - protein, 8 aa, Homo sapiens
  SITE (4)..(4): The peptide is optionally O-GlcNAcylated at this site
NPVSLPSL

SEQ ID NO: 28 - protein, 9 aa, Homo sapiens
  SITE (4)..(5): The peptide is optionally hexose-GlcNAcylated at one of these sites
PPSTSAAAL

SEQ ID NO: 29 - protein, 9 aa, Homo sapiens
  SITE (1)..(1): The peptide is optionally methylated or dimethylated at this site
  SITE (5)..(5): The peptide is optionally O-GlcNAcylated at this site
RPPITQSSL

SEQ ID NO: 30 - protein, 10 aa, Homo sapiens
  SITE (5)..(5): The peptide is optionally hexose-GlcNAcylated at this site
RPPQSSSVSL

SEQ ID NO: 31 - protein, 9 aa, Homo sapiens
  SITE (4)..(6): The peptide is optionally O-GlcNAcylated at one of these site
RPPSSSQQL

SEQ ID NO: 32 - protein, 10 aa, Homo sapiens
  SITE (5)..(5): The peptide is optionally O-GlcNAcylated at this site
RPPVTKASSF

SEQ ID NO: 33 - protein, 10 aa, Homo sapiens
  SITE (4)..(4): The peptide is optionally O-GlcNAcylated at this site
RPVTASITTM

SEQ ID NO: 34 - protein, 10 aa, Homo sapiens
  SITE (5)..(5): The peptide is optionally O-GlcNAcylated at this site
TPASSRAQTL

SEQ ID NO: 35 - protein, 9 aa, Homo sapiens
  SITE (4)..(4): The peptide is optionally O-GlcNAcylated at this site
TPASSSSAL

SEQ ID NO: 36 - protein, 9 aa, Homo sapiens
  SITE (4)..(4): The peptide is optionally O-GlcNAcylated at this site
TPISQAQKL

SEQ ID NO: 37 - protein, 9 aa, Homo sapiens
  SITE (4)..(4): The peptide is optionally O-GlcNAcylated at this site
TPVSSANMM

SEQ ID NO: 38 - protein, 9 aa, Homo sapiens
  SITE (4)..(4): The peptide is optionally O-GlcNAcylated at this site
VLTSNVQTI

SEQ ID NO: 39 - protein, 9 aa, Homo sapiens
  SITE (4)..(4): The peptide is optionally O-GlcNAcylated at this site
VPASSTSTL

SEQ ID NO: 40 - protein, 10 aa, Homo sapiens
  SITE (4)..(4): The peptide is optionally O-GlcNAcylated at this site
VPATHGQVTY

SEQ ID NO: 41 - protein, 8 aa, Homo sapiens
  SITE (3)..(5): The peptide is optionally O-GlcNAcylated at one of these sites
VPTTSSSL

SEQ ID NO: 42 - protein, 9 aa, Homo sapiens
  SITE (4)..(4): The peptide is optionally O-GlcNAcylated at this site
VPVSGTQGL

SEQ ID NO: 43 - protein, 9 aa, Homo sapiens
  SITE (4)..(4): The peptide is optionally O-GlcNAcylated at this site
VPVSNQSSL

SEQ ID NO: 44 - protein, 9 aa, Homo sapiens
  SITE (4)..(4): The peptide is optionally O-GlcNAcylated at this site
VPVSSASEL

SEQ ID NO: 45 - protein, 9 aa, Homo sapiens
  SITE (4)..(4): The peptide is optionally O-GlcNAcylated at this site
VPVSVGPSL

SEQ ID NO: 46 - protein, 9 aa, Homo sapiens
AAQERRVPR

SEQ ID NO: 47 - protein, 9 aa, Homo sapiens
HLFGYSWYK

SEQ ID NO: 48 - protein, 9 aa, Homo sapiens
YLSGADLNL

SEQ ID NO: 49 - protein, 9 aa, Homo sapiens
LLGPGRPYR

SEQ ID NO: 50 - protein, 9 aa, Homo sapiens
EIWTHSYKV

SEQ ID NO: 51 - protein, 9 aa, Homo sapiens
ALLAVGATK

SEQ ID NO: 52 - protein, 16 aa, Homo sapiens
WNRQLYPEWTEAQRLD

SEQ ID NO: 53 - protein, 9 aa, Homo sapiens
ALNFPGSQK

SEQ ID NO: 54 - protein, 9 aa, Homo sapiens
KTWGQYWQV

SEQ ID NO: 55 - protein, 9 aa, Homo sapiens
ITDQVPFSV

SEQ ID NO: 56 - protein, 9 aa, Homo sapiens
IMDQVPFSV

SEQ ID NO: 57 - protein, 9 aa, Homo sapiens
YLEPGPVTA

SEQ ID NO: 58 - protein, 10 aa, Homo sapiens
VLYRYGSFSV

SEQ ID NO: 59 - protein, 9 aa, Homo sapiens
LIYRRRLMK

SEQ ID NO: 60 - protein, 9 aa, Homo sapiens
KIFGSLAFL

SEQ ID NO: 61 - protein, 9 aa, Homo sapiens
VLRENTSPK

SEQ ID NO: 62 - protein, 14 aa, Homo sapiens
LLKYRAREPVTKAE

SEQ ID NO: 63 - protein, 9 aa, Homo sapiens
EADPTGHSY

SEQ ID NO: 64 - protein, 9 aa, Homo sapiens
SLFRAVITK

SEQ ID NO: 65 - protein, 9 aa, Homo sapiens
EVDPIGHLY

SEQ ID NO: 66 - protein, 15 aa, Homo sapiens
TSYVKVLHHMVKISG

SEQ ID NO: 67 - protein, 9 aa, Homo sapiens
GLYDGMEHL

SEQ ID NO: 68 - protein, 9 aa, Homo sapiens
AAGIGILTV

SEQ ID NO: 69 - protein, 23 aa, Homo sapiens
RNGYRALMDKSLHVGTQCALTRR

SEQ ID NO: 70 - protein, 20 aa, Homo sapiens
  MISC_FEATURE (12)..(12): The amino acid at the noted position is optionally phosphorylated
VPNAPPAYEKLSAEQSPPPY

SEQ ID NO: 71 - protein, 12 aa, Homo sapiens
  MISC_FEATURE (11)..(11): The amino acid at the noted position is optionally phosphorylated
PNAPPAYEKLSA

SEQ ID NO: 72 - protein, 12 aa, Homo sapiens
  MISC_FEATURE (10)..(10): The amino acid at the noted position is optionally phosphorylated
NAPPAYEKLSAE

SEQ ID NO: 73 - protein, 9 aa, Homo sapiens
  MISC_FEATURE (9)..(9): The amino acid at the noted position is optionally phosphorylated
APPAYEKLS

SEQ ID NO: 74 - protein, 12 aa, Homo sapiens
  MISC_FEATURE (9)..(9): The amino acid at the noted position is optionally phosphorylated
APPAYEKLSAEQ

SEQ ID NO: 75 - protein, 15 aa, Homo sapiens
  MISC_FEATURE (9)..(9): The amino acid at the noted position is optionally phosphorylated
APPAYEKLSAEQSPP

SEQ ID NO: 76 - protein, 16 aa, Homo sapiens
  MISC_FEATURE (9)..(9): The amino acid at the noted position is optionally phosphorylated
APPAYEKLSAEQSPPP

SEQ ID NO: 77 - protein, 17 aa, Homo sapiens
  MISC_FEATURE (9)..(9): The amino acid at the noted position is optionally phosphorylated
APPAYEKLSAEQSPPPY

SEQ ID NO: 78 - protein, 9 aa, Homo sapiens
  MISC_FEATURE (8)..(8): The amino acid at the noted position is optionally phosphorylated
PPAYEKLSA

SEQ ID NO: 79 - protein, 12 aa, Homo sapiens
  MISC_FEATURE (8)..(8): The amino acid at the noted position is optionally phosphorylated
PPAYEKLSAEQS

SEQ ID NO: 80 - protein, 9 aa, Homo sapiens
  MISC_FEATURE (7)..(7): The amino acid at the noted position is optionally phosphorylated
PAYEKLSAE

SEQ ID NO: 81 - protein, 12 aa, Homo sapiens
  MISC_FEATURE (7)..(7): The amino acid at the noted position is optionally phosphorylated
PAYEKLSAEQSP

SEQ ID NO: 82 - protein, 12 aa, Homo sapiens
  MISC_FEATURE (6)..(6): The amino acid at the noted position is optionally phosphorylated
AYEKLSAEQSPP

SEQ ID NO: 83 - protein, 12 aa, Homo sapiens
  MISC_FEATURE (5)..(5): The amino acid at the noted position is optionally phosphorylated
YEKLSAEQSPPP

SEQ ID NO: 84 - protein, 10 aa, Homo sapiens
ASGPGGGAPR

SEQ ID NO: 85 - protein, 16 aa, Homo sapiens
AQYIKANSKFIGITEL

SEQ ID NO: 86 - protein, 9 aa, Homo sapiens
RLSNRLLLR

SEQ ID NO: 87 - protein, 16 aa, Homo sapiens
AQNILLSNAPLGPQFP

SEQ ID NO: 88 - protein, 11 aa, Homo sapiens
SSDYVIPIGTY

SEQ ID NO: 89 - protein, 13 aa, Homo sapiens
SDAEKSDICTDEY

SEQ ID NO: 90 - protein, 9 aa, Homo sapiens
KCDICTDEY

SEQ ID NO: 91 - protein, 10 aa, Homo sapiens
DYMDGTMSQV

SEQ ID NO: 92 - protein, 21 aa, Homo sapiens [sequence omitted]

SEQ ID NO: 93 - protein, 10 aa, Human herpesvirus 5
TPRVTGGGAM

SEQ ID NO: 94 - protein, 9 aa, Human herpesvirus 4
RPPIFIRRL

SEQ ID NO: 95 - protein, 9 aa, Influenza A virus
QPEWFRNVL

SEQ ID NO: 96 - protein, 15 aa, Clostridium tetani
QYIKANSKFIGITEL

SEQ ID NO: 97 - protein, 16 aa, Artificial Sequence
  Artificially synthesized tetanus peptide
AQYIKANSKFIGITEL

SEQ ID NO: 98 - protein, 13 aa, Artificial Sequence
  Artificially synthesized PADRE peptide
AKFVAAWTLKAAA

SEQ ID NO: 99 - protein, 6 aa, Artificial Sequence
  Artificially synthesized peptide fragment
  misc_feature (1)..(1): Xaa can be any naturally occurring amino acid
  misc_feature (6)..(6): Xaa can be any naturally occurring amino acid
XTQSSX

SEQ ID NO: 100 - protein, 9 aa, Artificial Sequence
  Artificially synthesized peptide fragment
  misc_feature (4)..(4): Xaa can be any naturally occurring amino acid
  misc_feature (9)..(9): Xaa can be any naturally occurring amino acid
RPPXTQSSX

SEQ ID NO: 101 - protein, 9 aa, Artificial Sequence
  Artificially synthesized peptide fragment
  misc_feature (1)..(1): Xaa can be Leu or Ile
  misc_feature (9)..(9): Xaa can be Leu or Ile
XPVSSHNSX

SEQ ID NO: 102 - protein, 5 aa, Artificial Sequence
  Artificially synthesized peptide fragment
  misc_feature (5)..(5): Xaa can be any naturally occurring amino acid
SHNSX

SEQ ID NO: 103 - protein, 4 aa, Artificial Sequence
  Artificially synthesized peptide fragment
  misc_feature (1)..(1): Xaa can be any naturally occurring amino acid
XPVS

(19) United States
(12) Patent Application Publication
(10) Pub. No.: US 2019/0015494 A1
(43) Pub. Date: Jan. 17, 2019

(54) IDENTIFICATION OF CLASS I MHC ASSOCIATED GLYCOPEPTIDES AS TARGETS FOR CANCER IMMUNOTHERAPY

(71) Applicants: University of Virginia Patent Foundation, Charolttesville, VA (US); The University of Birmingham, Birmingham (GB)

(72) Inventors: Donald F. Hunt, Charlottesville (US); Jeffrey Shabanowitz, Charlottesville (US); Stacy Alyse Malaker, Charlottesville (US); Mark Cobbold, Winchester (US); Sarah Penny, Birmingham (GB)

(73) Assignees: University of Virginia Patent Foundation, Charlottesville (US); The University of Birmingham, Birmingham (GB)

(21) Appl. No.: 15/750,607

(22) PCT Filed: Aug. 5, 2016
(86) PCT No.: PCT/US2016/045852

§ 371 Date: Feb. 6, 2018

Related U.S. Application Data
(60) Provisional application No. 62/202,359, filed on Aug. 7, 2015.

(51) Int. Cl. A61K 39/00; A61K 38/03; A61K 38/19; A61P 35/02; A61K 47/54; A61K 39/395

(52) U.S. Cl. CPC A61K 39/001114; A61K 38/03; A61K 38/193; A61P 35/02; A61K 39/39; A61K 39/39558; A61K 39/001141; A61K 39/001134; A61K 2039/572; A61K 47/549

(57) ABSTRACT

Provided are compositions that include one or more peptides, wherein each peptide is at least 8 amino acids long and has an amino acid sequence as set forth in any of SEQ ID NOs: 1-45. Also provided are in vitro populations of dendritic cells that include the disclosed compositions, in vitro populations of CD8⁺ T cells capable of being activated upon being brought into contact with the disclosed populations of dendritic cells, antibodies or antibody-like molecules that specifically bind to complexes of MHC class I molecules and the disclosed peptides, methods for treating and/or preventing cancer such as leukemia using the disclosed compositions and/or populations, methods for making cancer vaccines using the disclosed compositions, methods for screening target peptides for inclusion in an immunotherapy composition, methods for determining a prognosis of a leukemia patient, and kits that include at least one of the disclosed peptides.

### CROSS-REFERENCE TO RELATED APPLICATION

This application claims the benefit of U.S. Provisional Patent Application Ser. No. 62/202,359, filed Aug. 7, 2015, the disclosure of which is incorporated herein by reference in its entirety.

### GRANT STATEMENT

This invention was made with government support under Grant Nos. AI033993 and GM037537 awarded by The National Institutes of Health. The government has certain rights in the invention.

### REFERENCE TO SEQUENCE LISTING

The Sequence Listing associated with the instant disclosure has been electronically submitted to the United States Patent and Trademark Office as International Receiving Office as a 25 kilobyte ASCII text file created on Aug. 5, 2016 and entitled “3062_12_PCT_ST25.txt”. The Sequence Listing submitted via EFS-Web is hereby incorporated by reference in its entirety.

### TECHNICAL FIELD

The presently disclosed subject matter relates to diagnostics and therapeutics. In particular, it relates to immunotherapies and diagnostics in the context of proliferative diseases such as but not limited to cancer.

### BACKGROUND

Cells in the human body communicate their health status to the immune system by degrading cellular proteins and presenting fragments of each on the cell surface in association with class I MHC proteins (Blum et al., 2013). Cytotoxic T-lymphocytes (CTL; CD8⁺ T-cells) scrutinize the class I MHC molecules on the cell surface, sample the protein fragments (peptides) being presented, and kill those cells that express new peptides as a result of viral, bacterial and parasitic infection, tissue transplantation and cellular transformation (cancer). Tumor antigens recognized by T-cells are the subject of a recent review (Coule et al., 2014).

Dysregulation of protein kinase and phosphatase activities, normally tightly controlled, are known to play prominent roles in the hallmark traits of cancer (Hanahan & Weinberg, 2011; Giancotti, 2014). These traits include sustained proliferative signaling, evasion of growth suppressors, resistance to apoptotic signals, unlimited replicative potential, induction of angiogenesis, activation of invasion and metastasis, reprogramming of energy metabolism, and eventual evasion of the immune system.

Class I MEW phosphopeptides, identified in earlier work on leukemia (see PCT International Patent Application Publication No. WO 2014/036562, which corresponds to U.S. Patent Application Publication No. 2015/0224182), elicit pre-existing, central-memory, T-cell-recall responses in multiple, healthy blood donors (Cobbold et al., 2013). These recall responses to phosphopeptide antigens are absent in some leukemia patients and correlate with clinical outcome. The response is restored following allogenic stem cell transplantation. These results suggested that class I MHC phosphopeptides derived from dysregulated cell signaling pathways in cancer are targets of immune surveillance in humans and, therefore, are attractive candidates for future cancer immunotherapy strategies.

O-GlcNAcylation is another process involved in the dysregulation of cell signaling pathways in cancer (Wells et al., 2004; Hart et al., 2011; Slawson & Hart, 2011; De Queiroz et al., 2014; Hart, 2014). Here, the enzyme O-GlcNAc transferase (OGT) adds the monosaccharide β-N-acetylglucosamine (derived from glucose) to Ser and Thr residues on a large fraction of proteins in both the cytoplasm and nucleus of the cell. Since the donor molecule for this process is UDP-GlcNAc that is produced in the hexose biosynthetic pathway, O-GlcNAcylation functions as a nutrient sensor and regulates numerous cell signaling pathways by blocking and unblocking phosphorylation sites (see e.g., Slawson & Hart, 2011; De Queiroz et al., 2014). GlcNAcylation sites are usually identical to, or in close proximity with, those sites that are phosphorylated, and OGT often exists in complex with a phosphatase that allows it to replace phosphate with O-GlcNAc (Wells et al., 2004). Removal of O-GlcNAc modifications is accomplished by the enzyme O-GlcNAcase (OGA). Since aberrant O-GlcNAcylation correlates with augmented cancer cell proliferation, survival, invasion, and metastasis (De Queiroz et al., 2014), it was hypothesized that O-GlcNAcylated class I MHC peptides that result from aberrant signaling should also be excellent candidates for use in cancer immunotherapy.

Research by Haurum et al. in the nineties showed that synthetic class I MEW O-GlcNAcylated peptides were: (a) suitable substrates for TAP-mediated transport into the endoplasmic reticulum (ER); (b) able to bind efficiently to class I MHC molecules; and (c) could elicit glycopeptide-specific T-cell responses in mice (Haurum et al., 1994; Haurum et al., 1995). X-ray structures on two of these synthetic peptide MHC complexes confirmed that the O-GlcNAc group was solvent-exposed and accessible to the T-cell receptor (Githero et al., 1999). The Haurum group also employed GlcNAc-β1-4 galactosyltransferase to selectively tag O-GlcNAcylated peptides isolated from human spleen with [³H]-UDP-Gal, and estimated that 0.1-1.0% of all MHC-associated peptides were O-GlcNAcylated (Haurum et al., 1999; Kastrup et al., 2000). However, a lack of an appropriate mass spectrometry technology made it impossible to characterize individual O-GlcNAcylated peptides.

The presently disclosed subject matter thus provides compositions and methods useful for identifying and using peptides, particularly O-GlcNAcylated peptides, for immunotherapy.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides compositions and methods useful for identifying I-GlcNAcylated peptides and for using them in immunotherapy. Additionally, the presently disclosed subject matter provides a number of novel O-GlcNAcylated peptides and methods for employing the same in immunotherapy.

Disclosed herein are several experimental approaches that led to the detection and sequencing of O-GlcNAcylated peptides in the complex mixture of non-glycosylated peptides presented by HLA A*0201, HLA B*0702, and HLA B*35 class I MEW molecules on the surface of primary leukemia samples and cell lines (see Tables 3-7).

The presently disclosed subject matter relates in some embodiments to post-translationally modified immunogenic therapeutic target peptides.

In some embodiments, the peptides of the presently disclosed subject matter are immunogenic. In some embodiments, the peptides are useful as vaccines.

In some embodiments, target peptides can be modified and analogs can be synthesized that retain their ability to stimulate a particular immune response but which also gain one or more beneficial features, such as those described herein below. In some embodiments, the presently disclosed peptides are useful for treating and/or vaccinating against multiple cancer types. In some embodiments, the presently disclosed peptides are useful against leukemia.

In some embodiments, substitutions can be made in the target peptides at residues known to interact with the MEC molecule. Such substitutions can have the effect of increasing the binding affinity of the target peptides for the MHC molecule and can also increase the half-life of the target peptide-MHC complex, the consequence of which is that the substituted target peptide is a more potent stimulator of an immune response than is the original target peptide.

Additionally, in some embodiments the substitutions have no effect on the immunogenicity of the target peptide per se, but rather prolong its biological half-life and/or prevent it from undergoing spontaneous alterations which might otherwise negatively impact on the immunogenicity of the peptide.

One of ordinary skill in the art will appreciate that the peptides of the presently, disclosed subject matter can have differing levels of immunogenicity, MHC binding, and/or ability to elicit CTL responses against cells displaying a native target peptide (e.g., on the surface of a tumor cell).

When administered, the peptides can be administered singly or in combinations as a cocktail of peptides.

Thus, the presently disclosed subject matter provides in some embodiments compositions comprising at least or about 1, 2, 3, 4, 5, 6, 7, 8, 9, 10, or more synthetic target peptides. In some embodiments, each synthetic target peptide is about or at least 8, 9, 10, 11, 12, 13, 14, or 15 amino acids long and comprises an amino acid sequence as set forth in any of SEQ ID NOs: 1-45. In some embodiments, the disclosed compositions stimulate T cell-mediated immune responses to at least one of the synthetic target peptides. In some embodiments, at least one of the synthetic target peptides comprises a substitution of a serine residue with a homo-serine residue. In some embodiments, at least one of the synthetic target peptides is an O-GlcNAcylated peptide or a mimetic thereof. In some embodiments, at least one of the synthetic target peptides is a methylated peptide or a mimetic thereof. In some embodiments, at least one of the synthetic target peptides is a peptide that is both O-GlcNAcylated and methylated. In some embodiments, at least one of the synthetic target peptides comprises an O-GlcNAcylated serine and a methylated arginine. In some embodiments, the composition is immunologically suitable for administration to a leukemia patient. In some embodiments, the composition comprises at least 5, 10, 15, or 20 different target peptides. In some embodiments, at least one of the synthetic target peptides is capable of binding to an MHC class I molecule of the HLA-A*0201 allele, a B*0702 allele, or a B*35 allele.

In some embodiments, the composition is capable of increasing the 5-year survival rate of leukemia patients treated with the composition by at least 20 percent relative to average 5-year survival rates that could have been expected without treatment with the composition. In some embodiments, the composition is capable of increasing the survival rate of leukemia patients treated with the composition by at least 20 percent relative to a survival rate that could have been expected without treatment with the composition. In some embodiments, the composition is capable of increasing the treatment response rate of leukemia patients treated with the composition by at least 20 percent relative to a treatment response rate that could have been expected without treatment with the composition. In some embodiments, the composition is capable of increasing the overall median survival of patients of leukemia patients treated with the composition by at least two months relative to an overall median survival that could have been expected without treatment with the composition.

In some embodiments, the disclosed compositions further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

In some embodiments, the disclosed compositions further comprise an adjuvant. In some embodiments the adjuvant is selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof. In some embodiments, the presently disclosed compositions comprise a peptide capable of binding to an MEW class I molecule of the HLA A*0201 allele, the B*0702 allele, or the B*35 allele.

In some embodiments, the presently disclosed subject matter also provides compositions comprising at least one synthetic target peptide, wherein each synthetic target peptide is between 8 and 50 amino acids long, and comprises an amino acid sequence selected from the group consisting of SEQ ID NOs: 1-45. In some embodiments, at least one serine residue in at least one of the synthetic target peptides is replaced with a homo-serine. In some embodiments, the composition comprises at least 5, 10, 15, or 20 different peptides.

In some embodiments, the presently disclosed compositions further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP and TPS.

In some embodiments, the presently disclosed compositions further comprise an agent selected from the group consisting of anti-CTLA-4, vermurafenib, ipilimumab, dacarbazine, IL-2, temozolomide, imatinib, gefitinib, erlotinib, sunitinib, tyrphostins and telatinib. In some embodiments, the presently disclosed compositions further comprise darcarbazine, carmustine, and tamoxifen.

In some embodiments, the presently disclosed compositions further comprise an adjuvant, optionally an adjuvant selected from the group consisting of montanide ISA-51, QS-21, tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), incomplete Freunds adjuvant, complete Freunds adjuvant, mineral gels, aluminum hydroxide (Alum), lysolecithin, pluronic polyols, polyanions, peptides, oil emulsions, dinitrophenol, and diphtheria toxin (DT).

In some embodiments, at least one of the synthetic target peptides is O-GlcNAcylated. In some embodiments, at least one of the synthetic target peptides comprises the amino acid sequence set forth in any of SEQ ID NOs: 9, 13, 14, and 41, and further wherein the amino acid at the third position in the sequence is O-GlcNAcylated; and/or the amino acid sequence set forth in any of SEQ ID NOs: 1, 2, 4-20, 24-28, 31, 33, and 35-45, and further wherein the amino acid at the fourth position in the sequence is O-GlcNAcylated; and/or the amino acid sequence set forth in any of SEQ ID NOs: 2, 3, 9, 12, 14, 19, 21, 23, 28-32, 34, and 41, and further wherein the amino acid at the fifth position in the sequence is O-GlcNAcylated; and/or the amino acid sequence set forth in any of SEQ ID NOs: 2, 6, 23, and 31, and further wherein the amino acid at the sixth position in the sequence is 0-GlcNAcylated; and/or the amino acid sequence set forth in any of SEQ ID NOs: 22 and 25, and further wherein the amino acid at the seventh position in the sequence is O-GlcNAcylated. In some embodiments, at least one of the synthetic target peptides comprises the amino acid sequence set forth in SEQ ID NO: 2, optionally wherein the amino acid at the fourth, fifth or sixth position, or at both the fourth and fifth position, or at both the fifth and sixth position in the sequence is O-GlcNAcylated, and further optionally wherein the amino acid at the fourth, fifth or sixth position comprises a single GlcNAc moiety or a single hexose-GlcNAc moiety; or the amino acids at the fourth and fifth positions both comprise GlcNAc moieties or hexose-GlcNAc moieties; or the amino acids at the fifth and sixth positions both comprise GlcNAc moieties or hexose-GlcNAc moieties; or the amino acids at the fifth position comprises a hexose-GlcNAc moiety and the amino acid at the sixth position comprises a GlcNAc moiety. In some embodiments, at least one of the synthetic target peptides comprises the amino acid sequence set forth in SEQ ID NO: 3, optionally wherein the amino acid at the fifth position comprises an N-linked hexose-GlcNAc moiety. In some embodiments, at least one of the synthetic target peptides comprises the amino acid sequence set forth in SEQ ID NO: 12, optionally wherein the amino acid at the fourth position comprises a GlcNAc moiety or the amino acids at the fourth and fifth positions both comprise a GlcNAc moiety. In some embodiments, at least one of the synthetic target peptides comprises the amino acid sequence set forth in SEQ ID NO: 18, optionally wherein the amino acid at the fourth position comprises a GlcNAc moiety or a hexose-GlcNAc moiety. In some embodiments, at least one of the synthetic target peptides comprises the amino acid sequence set forth in SEQ ID NO: 19, optionally wherein the amino acid at the fourth position comprises a GlcNAc moiety, a hexose-GlcNAc moiety, or an acetyl-GlcNAc moiety; or the amino acids at both the fourth and fifth positions both comprise a GlcNAc moiety. In some embodiments, at least one of the synthetic target peptides comprises the amino acid sequence set forth in SEQ ID NO: 20, optionally wherein the amino acid at the fourth position comprises a GlcNAc moiety or a hexose-GlcNAc moiety. In some embodiments, at least one of the synthetic target peptides comprises the amino acid sequence set forth in SEQ ID NO: 21, optionally wherein the amino acid at the fifth position comprises a hexose-GlcNAc moiety. In some embodiments, at least one of the synthetic target peptides comprises the amino acid sequence set forth in SEQ ID NO: 22, optionally wherein the amino acid at the seventh position comprises an N-linked hexose-GlcNAc moiety. In some embodiments, at least one of the synthetic target peptides comprises the amino acid sequence set forth in SEQ ID NO: 25, optionally wherein the amino acid at the seventh position comprises a hexose-GlcNAc moiety or the amino acid at the fourth position comprises an asymmetric di-methyl moiety and the amino acid at the seventh position comprises a hexose-GlcNAc moiety. In some embodiments, at least one of the synthetic target peptides comprises the amino acid sequence set forth in SEQ ID NO: 28, optionally wherein the amino acid at the fourth position or the amino acid at the fifth position comprises an O-linked hexose-GlcNAc moiety. In some embodiments, at least one of the synthetic target peptides comprises the amino acid sequence set forth in SEQ ID NO: 29, optionally wherein the amino acid at the fifth position comprises a GlcNAc moiety, the amino acid at the first position comprises a mono-methyl moiety and the amino acid at the fifth position comprises a GlcNAc moiety, the amino acid at the first position comprises an asymmetric di-methyl moiety and the amino acid at the fifth position comprises a GlcNAc moiety, or the amino acid at the first position comprises an asymmetric di-methyl moiety and the amino acid at the fifth position comprises an acetyl-GlcNAc moiety. In some embodiments, at least one of the synthetic target peptides comprises the amino acid sequence set forth in SEQ ID NO: 30, optionally wherein the amino acid at the fifth position comprises an O-linked hexose-GlcNAc moiety. In some embodiments, the composition has the ability to stimulate a T cell mediated immune response to at least one of the synthetic target peptides.

In some embodiments, the presently disclosed subject matter provides compositions comprising synthetic target peptides and an adjuvant. In some embodiments, the synthetic target peptide is between 8 and 50 amino acids long and comprises the amino acid sequence set forth in any of SEQ ID NOs: 1-45. In some embodiments, the adjuvant is QS-21.

In some embodiments, the presently disclosed subject matter provides compositions that further comprise a pharmaceutically acceptable carrier, optionally a pharmaceutically acceptable carrier that is pharmaceutically acceptable for use in a human.

In some embodiments of the presently disclosed compositions, the compositions comprise a peptide capable of binding to an MHC class I molecule of the HLA A*0201 allele, the B*0702 allele, or the B*35 allele.

In some embodiments, the presently disclosed subject matter provides compositions comprising at least one synthetic target peptide, wherein each synthetic target peptide: (i) is between 8 and 50 amino acids long, and (ii) comprises an amino acid sequence selected from the group consisting of SEQ ID NOs: 1-45. Use, at least one serine residue in at least one of the synthetic target peptides is replaced with a homo-serine residue. In some embodiments, the composition comprises at least 5, 10, 15, or 20 different peptides. In some embodiments, the compositions further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS. In some embodiments, the compositions further comprise an agent selected from the group consisting of anti-CTLA-4 antibody, vermurafenib, ipilimumab, dacarbazine, IL-2, temozolomide, imatinib, gefitinib, erlotinib, sunitinib, tyrphostins, and telatinib. In some embodiments, the compositions further comprise darcarbazine, carmustine, and tamoxifen. In some embodiments, the composition further comprises an adjuvant. In some embodiments, at least one of the synthetic target peptides is O-GlcNAcylated. In some embodiments, at least one of the synthetic target peptides comprises: the amino acid sequence set forth in any of SEQ ID NOs: 9, 13, 14, and 41, and further wherein the amino acid at the third position in the sequence is O-GlcNAcylated; and/or the amino acid sequence set forth in any of SEQ ID NOs: 1, 2, 4-20, 24-28, 31, 33, and 35-45, and further wherein the amino acid at the fourth position in the sequence is O-GlcNAcylated; and/or the amino acid sequence set forth in any of SEQ ID NOs: 2, 3, 9, 12, 14, 19, 21, 23, 28-32, 34, and 41, and further wherein the amino acid at the fifth position in the sequence is O-GlcNAcylated; and/or the amino acid sequence set forth in any of SEQ ID NOs: 2, 6, 23, and 31, and further wherein the amino acid at the sixth position in the sequence is O-GlcNAcylated; and/or the amino acid sequence set forth in any of SEQ ID NOs: 22 and 25, and further wherein the amino acid at the seventh position in the sequence is O-GlcNAcylated. In some embodiments, at least one of the synthetic target peptides comprises the amino acid sequence set forth in SEQ ID NO: 2, and further wherein the amino acid at the fourth, fifth or sixth position, or at both the fourth and fifth positions, or at both the fifth and sixth positions in the sequence is O-GlcNAcylated. In some embodiments, the amino acid at the fourth, fifth or sixth position comprises a single GlcNAc moiety or a single hexose-GlcNAc moiety; or the amino acids at the fourth and fifth positions both comprise GlcNAc moieties or hexose-GlcNAc moieties; or the amino acids at the fifth and sixth positions both comprise GlcNAc moieties or hexose-GlcNAc moieties; or the amino acids at the fifth position comprises a hexose-GlcNAc moiety and the amino acid at the sixth position comprises a GlcNAc moiety. In some embodiments, at least one of the synthetic target peptides comprises the amino acid sequence set forth in SEQ ID NO: 3, and further wherein the amino acid at the fifth position comprises an N-linked hexose-GlcNAc moiety. In some embodiments, at least one of the synthetic target peptides comprises the amino acid sequence set forth in SEQ ID NO: 12, and further wherein the amino acid at the fourth position comprises a GlcNAc moiety or the amino acids at the fourth and fifth positions both comprise a GlcNAc moiety. In some embodiments, at least one of the synthetic target peptides comprises the amino acid sequence set forth in SEQ ID NO: 18, and further wherein the amino acid at the fourth position comprises a GlcNAc moiety or a hexose-GlcNAc moiety. In some embodiments, at least one of the synthetic target peptides comprises the amino acid sequence set forth in SEQ ID NO: 19, and further wherein the amino acid at the fourth position comprises a GlcNAc moiety, a hexose-GlcNAc moiety, or an acetyl-GlcNAc moiety; or the amino acids at the fourth and fifth positions both comprise a GlcNAc moiety. In some embodiments, at least one of the synthetic target peptides comprises the amino acid sequence set forth in SEQ ID NO: 20, and further wherein the amino acid at the fourth position comprises a GlcNAc moiety or a hexose-GlcNAc moiety. In some embodiments, at least one of the synthetic target peptides comprises the amino acid sequence set forth in SEQ ID NO: 21, and further wherein the amino acid at the fifth position comprises a hexose-GlcNAc moiety. Isem at least one of the synthetic target peptides comprises the amino acid sequence set forth in SEQ ID NO: 22, and further wherein the amino acid at the seventh position comprises an N-linked hexose-GlcNAc moiety. In some embodiments, at least one of the synthetic target peptides comprises the amino acid sequence set forth in SEQ ID NO: 25, and further wherein the amino acid at the seventh position comprises a hexose-GlcNAc moiety or the amino acid at the fourth position comprises an asymmetric di-methyl moiety and the amino acid at the seventh position comprises a hexose-GlcNAc moiety. In some embodiments, at least one of the synthetic target peptides comprises the amino acid sequence set forth in SEQ ID NO: 28, and further wherein the amino acid at the fourth position or the amino acid at the fifth position comprises an O-linked hexose-GlcNAc moiety. In some embodiments, at least one of the synthetic target peptides comprises the amino acid sequence set forth in SEQ ID NO: 29, and further wherein the amino acid at the fifth position comprises a GlcNAc moiety, the amino acid at the first position comprises a mono-methyl moiety and the amino acid at the fifth position comprises a GlcNAc moiety, the amino acid at the first position comprises an asymmetric di-methyl moiety and the amino acid at the fifth position comprises a GlcNAc moiety, or the amino acid at the first position comprises an asymmetric di-methyl moiety and the amino acid at the fifth position comprises an acetyl-GlcNAc moiety. In some embodiments, at least one of the synthetic target peptides comprises the amino acid sequence set forth in SEQ ID NO: 30, and further wherein the amino acid at the fifth position comprises an O-linked hexose-GlcNAc moiety. In some embodiments, the composition has the ability to stimulate a T cell mediated immune response to at least one of the synthetic target peptides. In some embodiments, the adjuvant is selected from the group consisting of montanide ISA-51, QS-21, tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), incomplete Freunds adjuvant, complete Freunds adjuvant, mineral gels, aluminum hydroxide (Alum), lysolecithin, pluronic polyols, polyanions, peptides, oil emulsions, dinitrophenol, and diphtheria toxin (DT).

In some embodiments, the presently disclosed subject matter provides compositions comprising a synthetic target peptide and an adjuvant. In some embodiments, the synthetic target peptide is between 8 and 50 amino acids long and comprises the amino acid sequence set forth in any of SEQ ID NOs: 1-45. In some embodiments, the adjuvant is QS-21.

In some embodiments, a composition of the presently disclosed subject matter further comprises a pharmaceutically acceptable carrier, optionally a pharmaceutically acceptable carrier that is pharmaceutically acceptable for use in a human.

In some embodiments of the presently disclosed compositions, the peptide is capable of binding to an MHC class I molecule of the HLA A*0201 allele, the B*0702 allele, or the B*35 allele.

In some embodiments, the presently disclosed subject matter provides an in vitro population of antigen presenting cells, optionally dendritic cells, comprising a presently disclosed composition, in some embodiments a composition comprising at least one target peptide comprising an amino acid sequence as set forth in any of SEQ ID NOs: 1-45.

In some embodiments, the presently disclosed subject matter also provides in vitro populations of CD8⁺ T cells capable of being activated upon being brought into contact with a population of antigen presenting cells, optionally dendritic cells, wherein the antigen presenting cells optionally comprise a composition as disclosed herein or a composition comprising at least one target peptide comprising an amino acid sequence as set forth in any of SEQ ID NOs: 1-45. In some embodiments, the in vitro population of CD8⁺ T cells is capable of being activated upon being brought into contact with a complex of an MHC class I molecule and a peptide comprising an amino acid sequence as set forth in any of SEQ ID NOs: 1-45.

In some embodiments, the presently disclosed subject matter also provides antibodies or antibody-like molecules that specifically bind to a complex of an MHC class I molecule and a peptide comprising an amino acid sequence as set forth in one or more of SEQ ID NOs: 1-45. In some embodiments, the peptide is O-GlcNAcylated and/or methylated, and wherein the antibody or antibody-like molecule does not substantially cross react with the corresponding non-O-GlcNAcylated and/or non-methylated peptide. In some embodiments, the antibody or antibody-like molecule is a member of the immunoglobulin superfamily. In some embodiments, the antibody or antibody-like molecule comprises a binding member selected from the group consisting of an Fab, Fab′, F(ab′)2, Fv, and a single-chain antibody. In some embodiments, the antibody or antibody-like molecule of the presently disclosed subject matter is conjugated to a therapeutic agent, optionally a therapeutic agent selected from the group consisting of an alkylating agent, an antimetabolite, a mitotic inhibitor, a taxoid, a vinca alkaloid, and an antibiotic. In some embodiments, the antibody or antibody-like molecule is a T cell receptor. In some embodiments, the antibody or antibody-like molecule is conjugated to a CD3 agonist.

In some embodiments, the presently disclosed subject matter also provides isolated polynucleotides encoding the presently disclosed antibodies or antibody-like molecules or a chain thereof, or the presently disclosed T cell receptors or a chain thereof.

In some embodiments, the presently disclosed subject matter provides vectors comprising the presently disclosed polynucleotides.

In some embodiments, the presently disclosed subject matter also provides recombinant host cells comprising the pd polynucleotids or the presently disclosed vectors. In some embodiments, the host cell is a T cell comprising a polynucleotide of the presently disclosed subject matter, wherein the polynucleotide encodes a T cell receptor of the presently disclosed subject matter or a chain thereof.

In some embodiments, the presently disclosed subject matter also provides methods for treating and/or preventing cancer. In some embodiments, the methods comprise administering to a subject in need thereof a therapeutically effective dose of a composition as disclosed herein or a composition comprising at least one target peptide comprising an amino acid sequence as set forth in any of SEQ ID NOs: 1-45 in combination with a pharmaceutically acceptable carrier.

In some embodiments, the presently disclosed subject matter also provides methods for treating and/or preventing leukemia. In some embodiments, the presently disclosed methods comprise administering to a subject in need thereof a therapeutically effective dose of a composition of the presently disclosed subject matter or a composition comprising at least one target peptide comprising an amino acid sequence as set forth in any of SEQ ID NOs: 1-45 in combination with a pharmaceutically acceptable carrier.

In some embodiments, the presently disclosed subject matter also provides methods for treating and/or preventing cancer comprising administering to a subject in need thereof a therapeutically effective dose of an in vitro population of APCs, optionally dendritic cells, as disclosed herein in combination with a pharmaceutically acceptable carrier.

In some embodiments, the presently disclosed subject matter also provides methods for treating and/or preventing cancer comprising administering to a subject in need thereof a therapeutically effective dose of an in vitro population of CD8⁺ T cells as disclosed herein in combination with a pharmaceutically acceptable carrier.

In some embodiments, the presently disclosed subject matter also provides methods for treating and/or preventing cancer comprising administering to a subject in need thereof a therapeutically effective dose of an antibody or antibody-like molecule of the presently disclosed subject matter, a T cell receptor of the presently disclosed subject matter, or a host cell of the presently disclosed subject matter in combination with a pharmaceutically acceptable carrier.

In some embodiments, the presently disclosed subject matter also provides methods for making a cancer vaccine. In some embodiments, the presently disclosed methods comprise combining a composition as disclosed herein with an adjuvant, optionally an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

In some embodiments, the presently disclosed subject matter also provides methods for screening target peptides for inclusion in a composition of the presently disclosed subject matter or for use in a method of using a composition of the presently disclosed subject matter. In some embodiments, the presently disclosed methods comprise (a) administering the target peptide to a human; (b) determining whether the target peptide is capable of inducing a target peptide-specific memory T cell response in the human; and (c) selecting the target peptide for inclusion in the composition or for use in the method of using the composition if the target peptide elicits a memory T cell response in the human.

In some embodiments, the presently disclosed subject matter also provides methods for determining a prognosis of a leukemia patient. In some embodiments, the methods comprise (a) administering to the patient a target peptide comprising an amino acid sequence as set forth in any of SEQ ID NOs: 1-45, wherein the target peptide is associated with the patient's leukemia; (b) determining whether the target peptide is capable of inducing a target peptide-specific memory T cell response in the patient; and (c) determining that the patient has a better prognosis if the patient mounts a memory T cell response to the target peptide than if the patient did not mount a memory T cell response to the target peptide.

In some embodiments, the presently disclosed subject matter also provides kits comprising at least one target peptide composition comprising at least one target peptide comprising an amino acid sequence as set forth in any of SEQ ID NOs: 1-45 and a cytokine and/or an adjuvant. In some embodiments, the presently disclosed kits comprise at least 2, 3, 4, or 5 target peptide compositions. In some embodiments, the at least one target peptide composition is one of the presently disclosed compositions. In some embodiments, the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF). In some embodiments, the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT). In some embodiments, the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT). In some embodiments, the presently disclosed kits further comprise at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS. In some embodiments, the at least one target peptide comprises an amino acid sequence as set forth in any of SEQ ID NOs: 1-45.

Various aspects and embodiments of the presently disclosed subject matter are described in further detail below.

These and other aspects and embodiments which will be apparent to those of skill in the art upon reading the specification provide the art with immunological tools and agents useful for diagnosing, prognosing, monitoring, and/or treating human cancers.

### BRIEF DESCRIPTION OF THE DRAWINGS

FIGS. 1A-1E depict the results of the identification of MHC class I associated glycopeptides on primary leukemia cells. FIG. 1A is a Higher Energy Collision Dissociation (HCD) mass spectrum of exemplary peptide IPVsSHNSL (SEQ ID NO: 19). Fragment ions that defined the complete amino acid sequence for the first O-GlcNAcylated peptide detected in ALL are labeled as b and y. Those that had lost the O-GlcNAc moiety are labeled with an asterisk. FIGS. 1B and 1C are fingerprint ions in the HCD spectra of O-GlcNAcylated (FIG. 1B) and O-GalNAcylated (FIG. 1C) peptides. Relative abundances of fragment ions derived from secondary fragmentation of the oxonium ion at m/z 204 are substantially different for O-GlcNAcylated and O-GalNAcylated peptides. FIG. 1D is a distribution of 36 HLA-B*0702-restricted glycopeptides among the different leukemia and healthy cells analyzed. ALL: acute lymphoblastic leukemia; Healthy cells: healthy donor tonsil/spleen cells; LCL: lymphoblastoid cell line; AML: acute myeloid leukemia; CLL: chronic lymphocytic leukemia. FIG. 1E is a bar graph of the number of copies per cell of the O-GlcNAcylated peptides identified on ALL versus healthy B cells for IPVsSHNSL (SEQ ID NO: 19), RPPItQSSL (SEQ ID NO: 29), and RPVtASITTM (SEQ ID NO: 33).

FIG. 2 is an Electron Transfer Dissociation (ETD) mass spectrum of exemplary peptide RPPItQSSL (SEQ ID NO: 29) containing an asymmetrically dimethylated Arg residue. The ETD spectrum for this peptide showed ions of type c (c3-c8) that defined the last 5 amino acids as XtQSSX (SEQ ID NO: 99). The CAD spectrum of this peptide contained an abundant ion corresponding to y8 at m/z 1045, so the dimethylated-Arg residue was at the N-terminus and the missing mass (194 Th) corresponded to two Pro residues. The sequence RPPXtQSSX (SEQ ID NO: 100) is uniquely found in RNA binding protein 27 as RPPITQSSL (SEQ ID NO: 29). Non-methylated and mono-methylated forms of this peptide were also detected and sequenced. Assignment of the two methyl groups on Arg as either symmetrical or asymmetrical was achieved by synthesizing the two possible structures and then recording ETD spectra on both. The insert shows that the symmetric structure accepted an electron into the protonated side chain of Arg and lost both CH₃N═C●-NHCH₃ (71 Th) and CH₃NH₂ (31 Th) whereas the asymmetric structure accepted an electron and lost both HN═C●-N(CH₃)₂ (71 Th) and HN(CH₃)₂ (45 Th). Thus, the RPPItQSSL (SEQ ID NO: 29) peptide was asymmetrically dimethylated on the side chain of the N-terminal Arg residue.

FIGS. 3A-3D depict the result of experiments showing healthy donor immunity to leukemia-associated post-translationally-modified neoantigens. FIG. 3A shows several FACS plots depicting the gating strategy used in the intracellular cytokine staining (ICS) protocol for TNFα, IFNγ, and IL-2 disclosed herein to determine healthy donor immunity to the O-GlcNAcylated peptides. Immunity to viral antigens was used as an internal control, for comparison. Pie charts depict the number of cells producing 1, 2, or all 3 cytokines tested. FIGS. 3B and 3C depict collated results of cytokine production (FIG. 3B) and degranulation (FIG. 3C) by healthy donor T cells in response to stimulation with post-translationally-modified leukemia neoantigens (APVsSKSSL (SEQ ID NO: 8); IPIsLHTSL (SEQ ID NO: 16); IPVsSHNSL (SEQ ID NO: 19); RPPItQSSL (SEQ ID NO: 29); Me-RPPItQSSL (monomethylated SEQ ID NO: 29); RPVtASITTM (SEQ ID NO: 33); TPIsQAQKL (SEQ ID NO: 36)). Three viral controls (TPRVTGGGAM (SEQ ID NO: 93; RPPIFIRRL (SEQ ID NO: 94); and QPEWFRNVL (SEQ ID NO: 95)) were included for comparison. FIG. 3D is a plot showing the correlation between the percentage of cells producing cytokine and degranulating for HD1. In FIGS. 3B and 3C, white boxes indicate that no response was detected; wide left to right hatched boxes indicate that signal was 1.5-2.0-fold over background; narrow broken right to left hatched boxes indicate that signal was 2.0-3.0-fold over background; narrow broken left to right hatched boxes indicate that signal was 3.0-10.0-fold over background; and wide right to left hatched boxes indicate that signal was greater than 10-fold over background.

FIGS. 4A-4F present the results of investigating T cell recognition of various forms of the peptide RPPITQSSL (SEQ ID NO: 29). FIGS. 4A and 4B are depictions of healthy donor immunity to the unmodified (RPPITQSSL; SEQ ID NO: 29), O-GlcNAcylated (RPPItQSSL; SEQ ID NO: 29), methylated (Me-RPPITQSSL; SEQ ID NO: 29), and both O-GlcNAcylated and methylated (Me-RPPItQSSL; SEQ ID NO: 29) peptide, measured by cytokine production (FIG. 4A) and degranulation (FIG. 4B). FIGS. 4C-4E are FACS plots of a T cell line grown from HD5 against the methylated RPPItQSSL (SEQ ID NO: 29) peptide. FIG. 4C shows the lymphocyte subpopulation (boxed area showing lymphocytes at 2.89%), FIG. 4D shows the percentage of the lymphocyte subpopulation that was CD8⁺ (85.2%), and FIG. 4E shows the percentage of cells recognizing the peptide assessed by overnight stimulation with the peptide and detection of CD137 and CD107a surface markers. The negative control accounted for 2.99% of the detections, and the T cell line targeting the Me-RPPItQSSL (SEQ ID NO: 29; methyl-RPPI(gT)QSSL) accounted for 21.3% of the detections. FIG. 4F is a bar graph of the results of this T cell line in a europium release killing assay to assess killing of autologous transformed B cells pulsed with different modifications of the peptide. Me-RPPItQSSL (SEQ ID NO: 29): methylated and O-GlcNAcylated peptide; Me-RPPITQSSL (SEQ ID NO: 29): methylated but non-GlcNAcylated peptide; RPPItQSSL (SEQ ID NO: 29): non-methylated O-GlcNAcylated peptide; RPPITQSSL (SEQ ID NO: 29) non-methylated and non-GlcNAcylated peptide. In FIGS. 4A and 4B, white boxes indicate that no response was detected; wide left to right hatched boxes indicate that signal was 1.5-2.0-fold over background; narrow broken right to left hatched boxes indicate that signal was 2.0-3.0-fold over background; and narrow broken left to right hatched boxes indicate that signal was 3.0-10.0-fold over background.

FIG. 5 is a depiction of a positional analysis of the O-GlcNAc peptides disclosed herein. It presents a Logoplot (Schneider & Stephens, 1990; Crooks et al., 2004) showing the probability of different amino acid residues at each position in the HLA-B*0702 O-GlcNAc peptides (see Tables 4 and 5).

FIG. 6 is a bar graph showing that the CD8 T cells producing cytokine in response to stimulation with various O-GlcNAcylated peptides (TPIsQAQKL (SEQ ID NO: 36); Me-RPPItQSSL (SEQ ID NO: 29); RPPItQSSL (SEQ ID NO: 29); IPVsSHNSL (SEQ ID NO: 19)) and viral-control peptides (QPEWFRNVL (SEQ ID NO: 95); RPPIFIRRL (SEQ ID NO: 94); TPRVTGGGAM (SEQ ID NO: 93) were divided into four subsets: central memory (CM; CD45RA⁻, CD27⁺; wide left to right hatched boxes); effector memory (EM; CD45RA⁻, CD2T; narrow right to left hatched boxes); terminally differentiated effector memory (TEMRA; CD45RA⁺, CD2T; wide right to left hatched boxes boxes); and naïve (CD45RA⁺, CD27; white boxes). HD1 T cells that produced cytokine in response to stimulation with peptides were stained with surface antibodies for phenotyping. As shown, comparing the proportions of cells of different phenotypes using ANOVA demonstrated that there were significantly fewer EM cells responding to O-GlcNAcylated peptides.

### BRIEF DESCRIPTION OF THE TABLES

Table 1 presents a summary of anchor residues for various HLA molecules.

Table 2 presents a listing of various tumor-associated peptides other than the O-GlcNAc peptides of the presently disclosed subject matter.

Tables 3-6 disclose 77 isoforms for 45 exemplary peptide sequences of the presently disclosed subject matter. As shown in Tables 3-6, multiple entries for the same exemplary peptide sequence show that for certain exemplary peptides, differences in the location and/or type of modification were observed, including but not limited to O-GlcNAc, di-O-GlcNAc, hexose-GlcNAc, di-hexose-GlcNAc, and GlcNAc+hexose-O-GlcNAc (see Tables 3-6). Small letters (s, t, n) in the peptide sequences presented in Tables 3-6 refer to Ser, Thr, and/or Asn residues that were modified by GlcNAcylation.

Table 7 discloses thirty-six peptides, several with multiple forms of glycosylation, that were isolated from HLA B*0702 Class I MHC molecules on several leukemias, cell lines, and healthy tissue.

### BRIEF DESCRIPTION OF THE SEQUENCE LISTING

A more complete understanding of the presently disclosed subject matter can be obtained by reference to the accompanying Sequence Listing, when considered in conjunction with the subsequent Detailed Description. The embodiments presented in the Sequence Listing are intended to be exemplary only and should not be construed as limiting the presently disclosed subject matter to the listed embodiments, in which SEQ ID NOs: 1-45 provide a listing of exemplary MHC class I target peptides associated with cancer. Additional details with respect to SEQ ID NOs: 1-45 are provided in Tables 3-7 herein below.

### DETAILED DESCRIPTION

Identifying neoantigens expressed by cells associated with cancer could be of value in providing methods for preventing and/or treating cancer, including leukemia. Cancer neoantigens are peptides presented on cancer cells, in some embodiments malignant cancer cells, but not healthy cells, and are targets of the spontaneously arising adaptive immunity to cancer. The response to these cancer antigens determines the ultimate fate of developing tumors (Gubin et al., 2015). Neoantigens are intrinsically linked to the cell's cancerous state; the best studied, to date, being mutational neoantigens, which contain nonsynonymous mutations in coding regions of expressed proteins (Coulie et al., 1995; Rooney et al., 2015; Linnemann et al., 2015; McGranahan et al., 2016). Some of the cancers with the best clinical responses to immunotherapies have some of the lowest mutational loads, particularly leukemias; therefore, these mutational neoantigens cannot account for the entire anti-cancer immune response (Lawrence et al., 2013; Ansell et al., 2015; Motzer et al., 2015). Another suggested source of neoantigens may be posttranslational modifications (PTMs) of proteins that occur in malignant and not healthy cells, particularly as dysregulated signaling is a hallmark of cancer (Hanahan & Weinberg, 2011). Indeed, a number of phosphorylated peptides have previously been identified as potent cancer antigens (Cobbold et al., 2013). Interestingly, immunity to these antigens was seen in healthy donors (HD), but lost in a subset of leukemia patients with poor clinical outcome and restored after stem cell transplant, suggesting a role for these antigens in the graft versus leukemia response. Dysregulation of cell signaling pathways in cancer is also caused by another PTM, β O-linked N-acetylglucosamine (O-GlcNAc), which is involved in cross-talk with phosphorylation (Wells et al., 2004; Slawson & Hart, 2011; Hart, 2014). As such, aberrant O-GlcNAcylation has been shown to correlate with augmented cancer cell proliferation, survival, invasion and metastasis (de Queiroz et al., 2014). It has also been shown that synthetic O-GlcNAc modified peptides can bind MHC class I complexes, and elicit glycopeptide-specific T cell responses in mice, with X-ray structures confirming that the O-GlcNAc group was solvent exposed and accessible to the T-cell receptor (Haurum et al., 1994; Haurum et al., 1995; Githero et al., 1999).

However, up until recently, limitations in proteomic technology made it impossible to characterize O-GlcNAcylated peptides from biological samples. Disclosed herein are three experimental approaches that allowed the detection and sequencing of O-GlcNAcylated peptides from a complex mixture of peptides presented by HLA-B*07:02 molecules on various primary leukemia samples. These methods allowed for the isolation of 36 unique glycopeptides in several different states of glycosylation and, surprisingly, methylation.

Also disclosed herein is the discovery that HDs have heterologous immunity to a number of these complex neoantigens and that T cells from these donors can specifically target and kill cells displaying only the modified peptide. Ultimately, these glycopeptide antigens can be employed in the design of novel cancer immunotherapeutics.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

The articles “a”, “an”, and “the” are used herein to refer to one or to more than one (i.e., to at least one) of the grammatical object of the article. By way of example, “an element” means one element or more than one element.

As used herein, the term “about” means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of in some embodiments ±20%, in some embodiments ±15%, in some embodiments ±10%, in some embodiments ±5%, in some embodiments ±1%, in some embodiments ±0.5%, in some embodiments ±0.1%, and in some embodiments less than ±0.1%.

As used herein, the terms “additional therapeutically active compound” and “additional therapeutic agent” refer to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” with respect to a compound, peptide, composition, etc. should be understood to mean providing a compound, peptide, composition, etc. of the presently disclosed subject matter or a prodrug of a compound, peptide, composition, etc. of the presently disclosed subject matter to a subject in need thereof, in some embodiments to ameliorate at least one symptom of a disease, disorder, or condition in the subject, to prevent the occurrence of at least one symptom of a disease, disorder, or condition in the subject, and/or to prevent the further development of at least one symptom of a disease, disorder, or condition in the subject.

As used herein, the term “aerosol” refers to suspension in the air. In particular, aerosol refers to the particlization or atomization of a formulation of the presently disclosed subject matter and its suspension in the air.

As used herein, an “agonist” is a composition of matter which, when administered to a mammal such as a human, enhances or extends a biological activity attributable to the level or presence of a target compound, peptide, composition, molecule of interest, etc. in the mammal.

An “antagonist” is a composition of matter which when administered to a mammal such as a human, inhibits a biological activity attributable to the level or presence of a target compound, peptide, composition, molecule of interest, etc. in the mammal.

As used herein, the phrase “alleviating a disease or disorder symptom” refers to reducing the severity of the symptom or the frequency with which such a symptom is experienced by a subject, or both. In some embodiments, “alleviating a disease or disorder symptom” refers to eliminating the symptom experienced by the subject.

As used herein, amino acids are represented by the full name thereof, by the three letter code corresponding thereto, and/or by the one-letter code corresponding thereto, as indicated in the following:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The phrase “amino acid” is used interchangeably with “amino acid residue”, and may refer to a free amino acid and/or to an amino acid residue of a peptide. It will be apparent from the context in which the term is used whether it refers to a free amino acid or a residue of a peptide.

Amino acids have the following general structure:

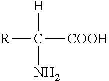

They may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” and the phrase “positively charged” as they relate to amino acids refer herein to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antigen” as used herein refers to a molecule that provokes an immune response in vitro and/or in vivo. This immune response can involve antibody production, the activation of specific immunologically-competent cells, or both. An antigen can be derived from an organism, a subunit of a protein, a killed or inactivated whole cell or lysate, or any other source to which an organism's immune system or a component thereof (e.g., an immune cell) can react.

The phrase “antigenic determinant” as used herein refers to that portion of an antigen that makes contact with a particular antibody (i.e., an epitope). When a protein, a fragment of a protein, or any other molecule is used to immunize a host animal, numerous regions of the antigen may induce the production of antibodies that bind specifically to a given region or three-dimensional structure on the protein. These regions or structures are referred to as antigenic determinants. An antigenic determinant may compete with the intact antigen (i.e., the “immunogen” used to elicit the immune response) for binding to an antibody.

The phrase “antimicrobial agents” as used herein refers to any naturally-occurring, synthetic, and/or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes but is not limited to antibacterial, antifungal, and antiviral agents.

As used herein, the phrases “antisense oligonucleotide” and “antisense nucleic acid” refer to a nucleic acid polymer, at least a portion of which is the reverse complement of a nucleic acid or subsequence thereof that is present in a normal cell or in an affected cell (e.g., a leukemic cell). “Antisense” refers particularly to the nucleic acid sequence of the non-coding strand of a double stranded DNA molecule encoding a protein, or to a sequence which is substantially homologous to the non-coding strand. As defined herein, in some embodiments an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro, ex vivo, in vivo, and/or in silico to bind preferentially to another compound (in some embodiments, to the peptides disclosed herein). Often, aptamers are nucleic acids or peptides because random sequences can be readily generated from nucleotides or amino acids (both naturally occurring and synthetically made) in large numbers, but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

As used herein, the phrase “binding partner” refers to a molecule capable of binding to another molecule. In some embodiments, binding partner bind to each other in vitro, ex vivo, in vivo, and/or under physiological conditions.

The term “biocompatible”, as used herein, refers to a material that does not elicit a substantial detrimental response in the host.

As used herein, the phrases “biologically active fragment” and “bioactive fragment” of polypeptides encompass natural and synthetic portions of full-length polypeptides that have one or more desirable characteristics of the full-length polypeptides, including but not limited to specific binding to their natural ligand(s) and/or performing desirable functions of the polypeptides.

The phrase “biological sample”, as used herein, refers to samples obtained and/or otherwise isolated from a subject, including, but not limited to, skin, hair, tissue, blood, plasma, cells, sweat, and urine.

The term “cancer”, as used herein, is defined as proliferation of cells whose unique trait—loss of normal growth control regulation—results in unregulated growth, lack of differentiation, local tissue invasion, and/or metastasis. Examples of cancers include but are not limited to, leukemia, melanoma, breast cancer, prostate cancer, ovarian cancer, uterine cancer, cervical cancer, skin cancer, pancreatic cancer, colorectal cancer, renal cancer, and lung cancer.

As used herein, the phrase “carrier molecule” refers to any molecule that is associated with and/or conjugated to an antigen of interest to thereby enable an immune response resulting in the production of antibodies specific to the native antigen.

The phrase “cell surface protein” means a protein found where at least part of the protein is exposed at the outer aspect of the cell membrane. Examples include growth factor receptors and other transmembrane molecules.

As used herein, the phrases “chemically conjugated” and “conjugating chemically” refer to linking an antigen to a carrier molecule. This linking can in some embodiments occur on the genetic level using recombinant technology, wherein a hybrid protein may be produced containing the amino acid sequences, or portions thereof, of both the antigen and the carrier molecule. This hybrid protein is in some embodiments produced by an oligonucleotide sequence encoding both the antigen and the carrier molecule, and/or portions thereof. This linking also in some embodiments includes covalent bonds created between the antigen and the carrier protein using other chemical reactions, such as but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkages between the antigen and the carrier molecule.

A “coding region” of a gene consists of the nucleotide residues of the coding strand of the gene and/or genetic locus and the nucleotides of the non-coding strand of the gene which are homologous with or complementary to, respectively, the coding region of an mRNA molecule which is produced by transcription of the gene. A “coding region” thus comprises the “open reading frame” of the genetic locus.

The phrase “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (in some embodiments at least 50%, 60%, 70%, 75%, 80%, 85%, 90%, 95%, 96%, 97%, 98%, 99%, or 100%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. The first region comprises a first portion and the second region comprises a second portion, whereby, when the first and second portions are arranged in an antiparallel fashion, in some embodiments at least about 50%, in some embodiments at least about 60%, in some embodiments at least about 70%, in some embodiments at least about 75%, in some embodiments at least about 80%, in some embodiments at least about 85%, in some embodiments at least about 90%, in some embodiments at least about 95%, in some embodiments at least about 96%, in some embodiments at least about 97%, in some embodiments at least about 98%, in some embodiments at least about 99%, and in some embodiments 100% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to any type of substance or agent that is commonly considered a drug, or a candidate for use as a drug, as well as combinations and mixtures of the above.

As used herein, the phrase “conservative amino acid substitution” is defined herein as an amino acid exchange within one of the following five groups:
A. Small aliphatic, nonpolar, or slightly polar residues: Ala, Ser, Thr, Pro, Gly;
B. Polar, negatively charged residues and their amides: Asp, Asn, Glu, Gln;
C. Polar, positively charged residues: His, Arg, Lys;
D. Large, aliphatic, nonpolar residues: Met, Leu, Ile, Val, Cys; and
E. Large, aromatic residues: Phe, Tyr, Trp.
   Thus, a conservative amino acid substitution includes a substitution of in some embodiments any small aliphatic, nonpolar, or slightly polar residue for any other small aliphatic, nonpolar, or slightly polar residues; in some embodiments any polar, negatively charged residue and its amide for any other polar, negatively charged residue and its amide; in some embodiments any polar, positively charged residue for any other polar, positively charged residue; in some embodiments any large, aliphatic, nonpolar residue for any other large, aliphatic, nonpolar residue; and/or in some embodiments any large, aromatic residue for any other large, aromatic residue.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the words “determine” and “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the detectable marker/reporter molecule in the presence of similar compounds that lack the detectable marker/reporter molecule. Detectable markers and reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization and/or altered light-scattering.

A “disease” is a state of health of an animal wherein the animal cannot maintain homeostasis, and wherein if the disease is not ameliorated then the animal's health would be expected to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular, and helical domains, and/or properties such as ligand binding, signal transduction, cell penetration, and the like. Specific examples of binding domains include, but are not limited to, DNA binding domains and ATP binding domains.

As used herein, an “effective amount” or “therapeutically effective amount” means an amount sufficient to produce a desired effect, such as ameliorating or alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is ameliorated and/or alleviated to a greater extent by one treatment relative to a second treatment to which it is being compared.

As used herein, the phrase “effector domain” refers to a domain capable of directly interacting with an effector molecule, chemical, and/or structure in the cytoplasm which is capable of regulating a biochemical pathway.

The term “elixir”, as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA, and mRNA) or a defined sequence of amino acids and the biological properties resulting therefrom. Thus, a genetic locus encodes a protein if transcription and translation of mRNA corresponding to that genetic locus produces the protein in a cell or other biological system. Both the coding strand (i.e., the nucleotide sequence of which is identical to the mRNA sequence and is usually provided in sequence listings), and the non-coding strand, used as the template for transcription of a gene or cDNA, can be referred to as encoding the protein or other product of that genetic locus or cDNA.

An “enhancer” is a DNA regulatory element that can increase the efficiency and/or rate of transcription, regardless of the distance or orientation of the enhancer relative to the start site of transcription.

The term “epitope” as used herein is defined as a small chemical group on an antigen molecule that can elicit and react with an antibody. An antigen can have one or more epitopes. Most antigens have many epitopes; i.e., they are multivalent. In general, epitopes are roughly five to eight amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95%, in some embodiments at least about 97%, and in some embodiments at least about 99%, by weight, of the total protein or total peptide in the preparation is the particular protein or peptide of interest.

A “fragment” or “segment” is a portion of an amino acid sequence (i.e., a subsequence) comprising at least one amino acid or a portion of a nucleic acid sequence comprising at least one nucleotide. The terms “fragment” and “segment” are used interchangeably herein.

As used herein, a “functional” biological molecule is a biological molecule in a form in which it exhibits a desirable property by which it can be characterized. A functional enzyme, for example, is one which exhibits the characteristic catalytic activity by which the enzyme is characterized.

As used herein, the term “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, the term “homology” is used synonymously with the term “identity”. Similarly, the term “homologous” is used synonymously with the term “identical”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., (BLAST and NBLAST) can be used.

The percent identity between two sequences can be determined using techniques similar to those described above, with or without allowing gaps. In calculating percent identity, typically exact matches are counted. In some embodiments, a percent identity is computed over a subsequence of the nucleic acid or amino acid, and in some embodiments the percent identity relates to comparing the full length sequence of a first nucleic acid or amino acid to either a subsequence of a second nucleic acid or amino acid or the full length sequence of the second nucleic acid or amino acid.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the phrase “immunizing a subject against an antigen” refers to administering to the subject a composition, a protein and/or peptide complex, a DNA encoding a protein and/or peptide complex, an antibody, or a DNA encoding a protein and/or peptide complex and/or an antibody, which elicits an immune response in the subject, and, for example, provides protection to the subject against a disease associated with and/or caused by the antigen or which prevents the function of the antigen.

The phrase “immunologically active fragments thereof” refers to a fragment of a polypeptide antigen (e.g., a peptide or subsequence thereof) comprising at least an epitope, which means that the fragment at least comprises 4, 5, 6, 7, 8, 9, or 10 contiguous amino acids from the sequence of the polypeptide antigen.

As used herein, the term “inhaler” refers both to devices for nasal and pulmonary administration of a drug, e.g., in solution, powder, and the like. For example, the term “inhaler” is intended to encompass a propellant driven inhaler, such as is used to administer antihistamine for acute asthma attacks, and plastic spray bottles, such as are used to administer decongestants.

As used herein “injecting” or “applying” includes administration of a compound (e.g., a peptide) of the presently disclosed subject matter by any number of routes including, but not limited to, topical, oral, buccal, intravenous, intramuscular, intraarterial, intramedullary, intrathecal, intraventricular, transdermal, subcutaneous, intraperitoneal, intranasal, enteral, topical, sublingual, vaginal, ophthalmic, pulmonary, and rectal routes of administration.

As used herein, an “instructional material” includes a publication, a recording, a diagram, and/or any other medium of expression which can be used to communicate the usefulness of the peptides of the presently disclosed subject matter in the kit for effecting alleviation of the various diseases or disorders recited herein. Alternately or in addition, the instructional material may describe one or more methods of ameliorating and/or alleviating the diseases or disorders in a cell or a tissue of a mammal. The instructional material of the kit of the presently disclosed subject matter may, for example, be affixed to a container which contains the identified compound(s) and/or peptide(s) of the presently disclosed subject matter or be shipped together with a container which contains the identified compound(s) and/or peptide(s). Alternatively or in addition, the instructional material may be shipped separately from the container with the intention that the instructional material and the compound(s) and/or peptide(s) be used cooperatively by the recipient.

By ‘interaction” between a first protein and a second protein is meant the interaction such as binding which is necessary for an event or process to occur, such as sperm-egg binding, fusion, and fertilization. In some embodiments, the interaction may be similar to a receptor-ligand type of binding or interaction.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA, DNA, and/or proteins, which naturally accompany it in the cell. The term therefore includes, for example, a recombinant DNA which is incorporated into a vector, into an autonomously replicating plasmid or virus, into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a molecule that specifically binds to a target molecule such as but not limited to a receptor. A “receptor” is a molecule that specifically binds to a ligand. In some embodiments, the attribution of a given molecule as being a “ligand” or a “receptor” is merely one of convenience in the event that the “receptor” can be a molecule that is not recognized as a “receptor” as that term might be understood with respect to cell biology and/or signal transduction.

As such, in some embodiments a ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or non-covalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The phrases “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. A level of expression can include rates of expression and can be measured in terms of the actual amount of an mRNA or protein present (sometimes referred to as the “abundance” of the mRNA or protein). Such assays are in some embodiments coupled with processes or systems to store and process information and to help quantify levels, signals, etc. and to digitize the information for use in comparing levels.

The phrase “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, and trans-mucosal administration of a drug is highly amenable to self-administration.

The phrase “nucleic acid” refers in some embodiments to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the phrase “nucleic acid” thus encompasses RNA as well as single-, double-, and/or triple-stranded DNA molecules and cDNAs. Furthermore, the terms “nucleic acid”, “DNA”, “RNA”, and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”. Similarly, non-coding sequences that are “upstream” or “5′ to” coding sequences are typically referred to as “upstream non-coding” or “5′ non-coding” sequences, and non-coding sequences that are “downstream” or “3′ to” coding sequences are typically referred to as “downstream non-coding” or “3′ non-coding” sequences

The phrase “nucleic acid construct” as used herein encompasses DNA and RNA sequences encoding a particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. It is well within the skill of one of ordinary skill in the art to determine each and every nucleotide sequence encoding a known amino acid sequence based on the redundancy of the genetic code. In some embodiments, nucleotide sequences that encode proteins and RNA can include one or more introns.

The term “oligonucleotide” typically refers to short polynucleotides, generally, no greater than about 10, 15, 20, 25, 30, 35, 40, 45, or 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” it is meant that a single-stranded or double-stranded nucleic acid comprises the two polynucleotides arranged within the nucleic acid in such a manner that at least one of the two polynucleotides is able to exert a physiological effect by which it is characterized upon the other. By way of example, a promoter operably linked to the coding region of a gene is able to promote transcription of the coding region. Other nucleic acid sequences that can be operably linked to other nucleic acid sequences include, but are not limited to origins of replication, enhancers, transcription terminators, polyadenylation signals and sites, ribosome entry sites, etc.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides. In some embodiments, a peptide of the presently disclosed subject matter is thus at least or about 6, 7, 8, 9, 10, 11, 12, 13, 14, or 15 amino acids long, including but not limited to at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids long. The peptides of the presently disclosed subject matter can in some embodiments also have a length that falls in the ranges of 6-8, 8-10, 9-12, 10-13, 11-14, 12-15, 15-20, 20-25, 25-30, 30-35, 35-40, and 45-50 amino acids. In some embodiments, exactly, about, or at least 1, 2, 3, 4, 5, 6, 7, 8, 9, 10, 11, 12, 13, 14, 15, 16, 17 or more of the amino acid residues within a recited sequence of a target peptide contains an O-GlcNAc moiety, a hexose-GlcNAc moiety, or any combination thereof.

The phrase “per application” as used herein refers to administration of a drug or compound to a subject.

The phrase “pharmaceutical composition” refers to a composition comprising at least one active ingredient, whereby the composition is amenable to administration for a specified, efficacious outcome to a mammal (for example, without limitation, a human). Those of ordinary skill in the art will understand and appreciate the techniques appropriate for determining whether an active ingredient has a desired efficacious outcome based upon the needs of the artisan.

As used herein, the phrase “pharmaceutically-acceptable carrier” means a chemical composition with which an appropriate compound and/or derivative can be combined and which, following the combination, can be used to administer the appropriate compound to a subject. In some embodiments, a pharmaceutically-acceptable carrier is pharmaceutically acceptable for use in a human, which means that the carrier is in some embodiments generally recognized as being safe (GRAS) for human consumption and/or administration. “Pharmaceutically acceptable” thus means physiologically tolerable, for either human or veterinary application.

As used herein, the phrase “physiologically acceptable” ester or salt means an ester or salt form of the active ingredient which is compatible with any other ingredients of the pharmaceutical composition, which is not deleterious to the subject to which the composition is to be administered.

As used herein, “pharmaceutical compositions” include formulations for human and veterinary use.

“Plurality” means at least two and, unless specifically limited herein, has no upper boundary.

As used herein, the term “polynucleotide” refers to a single strand or parallel and anti-parallel strands of a nucleic acid. Thus, a polynucleotide may be a single-stranded nucleic acid, a double-stranded nucleic acid, a triple-stranded nucleic acid, etc.

As used herein, the term “polypeptide” refers to a polymer composed of amino acid residues, related naturally occurring structural variants, and/or synthetic non-naturally occurring analogs thereof linked via peptide bonds, related naturally occurring structural variants, and synthetic non-naturally occurring analogs thereof.

As used herein, the phrase “synthetic peptides or polypeptides” refers to non-naturally occurring peptides and polypeptides. Synthetic peptides and polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene encodes a recombinant polynucleotide, produces a “recombinant polypeptide”.

A “recombinant polypeptide” is one which is produced upon expression of a recombinant polynucleotide.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include in some embodiments mammals, which in some embodiments can be a human.

As used herein, a “subject in need thereof” is a patient, animal, mammal, or human, who will benefit from the method of the presently disclosed subject matter.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

The phrase “substantially homologous nucleic acid sequence” refers to a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, in some embodiments a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine”, as used herein, is meant a composition which when inoculated into a subject has the effect of stimulating an immune response in the subject, which serves to fully or partially protect the subject against a condition, disease or its symptoms. In one aspect, the condition is conception. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### II. Target Peptides

The presently disclosed subject matter relates in some embodiments to post-translationally modified immunogenic therapeutic target peptides, e.g., O-GlcNAc peptides or other glycosylated peptides, for use in immunotherapy and diagnostic methods of using the target peptides, as well as methods of selecting the same to make compositions for immunotherapy, e.g., in vaccines and/or in compositions useful in adaptive cell transfer.

In some embodiments, the target peptides of the presently disclosed subject matter are post-translationally modified by being provided with one or more O-linked beta-N-acetylglucosamine (“O-GlcNAc”) moieties, one or more hexose-GlcNAc moieties, or any combination thereof (collectively referred to herein as “O-GlcNAc peptides”). In some embodiments, a target peptide of the presently disclosed subject matter is and/or comprises a mimetic of an O-GlcNAc peptide or amino acid. Exemplary, non-limiting glycosaminoglycan mimetics that can be employed in the presently disclosed subject matter are disclosed, for example, in U.S. Pat. No. 8,912,149, the disclosure of which is incorporated herein by reference in its entirety.

In some embodiments, a target peptide of the presently disclosed subject matter is and/or comprises a mimetic of a methylated peptide or amino acid.

The target peptides of the presently disclosed subject matter are in some embodiments not the entire proteins from which they are derived (i.e., are fragments and/or subsequences of larger polypeptides). They are in some embodiments from 8 to 50 contiguous amino acid residues of the native human protein. In some embodiments, they can contain exactly, about, or at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. The peptides of the presently disclosed subject matter can in some embodiments also have a length that falls in the ranges of 8-10, 9-12, 10-13, 11-14, 12-15, 15-20, 20-25, 25-30, 30-35, 35-40, and 45-50 amino acids. In some embodiments, exactly, about, or at least 1, 2, 3, 4, 5, 6, 7, 8, 9, 10, 11, 12, 13, 14, 15, 16, 17 or more of the amino acid residues within a recited sequence of a target peptide contains one or more O-GlcNAc moieties, one or more hexose-GlcNAc moieties, or any combination thereof.

Target peptides can be modified and analogs can be synthesized that retain their ability to stimulate a particular immune response but which also gain one or more beneficial features, such as those described herein below. Thus, a particular target peptide can, for example, have use for treating and vaccinating against multiple cancer types.

Substitutions can be made in the target peptides at residues known to interact with the MHC molecule. Such substitutions can have the effect of increasing the binding affinity of the target peptides for the MHC molecule and can also increase the half-life of the target peptide-MHC complex, the consequence of which is that the substituted target peptide is a more potent stimulator of an immune response than is the original target peptide.

Additionally, in some embodiments the substitutions have no effect on the immunogenicity of the target peptide per se, but rather prolong its biological half-life and/or prevent it from undergoing spontaneous alterations which might otherwise negatively impact on the immunogenicity of the peptide.

The target peptides disclosed herein can have differing levels of immunogenicity, MHC binding, and ability to elicit CTL responses against cells displaying a native target peptide (e.g., on the surface of a tumor cell).

An O-GlcNAc peptide as disclosed herein is in some embodiments modified such that its immunogenicity and/or its binding is enhanced. In some embodiments, the modified target peptide binds to an MHC class I molecule about or at least 10%, 20%, 30%, 40%, 50%, 60%, 70%, 80%, 90%, 95%, 100%, 110%, 125%, 150%, 175%, 200%, 225%, 250%, 275%, 300%, 350%, 375%, 400%, 450%, 500%, 600%, 700%, 800%, 900%, 1000%, 10,000%, 100,000%, 1,000,000%, or more tightly than its native counterpart.

However, given the exquisite sensitivity of the T cell receptor, it cannot be foreseen whether such enhanced binding and/or immunogenicity will render a modified target peptide still capable of inducing an activated CTL that will cross react with the native target peptide being displayed on the surface of a tumor. Indeed, it is disclosed herein that the binding affinity of a target peptide does not predict its functional ability to elicit a T cell response.

Target peptides of the presently disclosed subject matter can in some embodiments be mixed together to form a cocktail. The target peptides can be in an admixture, or they can be linked together in a concatamer and/or in other arrangement as a single molecule. Linkers between individual target peptides can be used; these can, for example, be formed in some embodiments by any 10 to 20 amino acid residues. The linkers can be random sequences, or they can be optimized for degradation by dendritic cells.

In certain specified positions, a native amino acid residue in a native human protein can be altered to enhance its binding to an MHC class I molecule. These occur in “anchor” positions of the target peptides, often in positions 1, 2, 3, 9, or 10. Valine, alanine, lysine, leucine tyrosine, arginine, phenylalanine, proline, glutamic acid, threonine, serine, aspartic acid, tryptophan, and methionine can also be used as improved anchoring residues. Anchor residues for different HLA molecules are listed below in Table 1.

TABLE 1
Anchor Residues for HLA Molecules

| HLA Type | Residue Position | Anchor Residue(s) |
|---|---|---|
| HLA A*0201 | 2 | L, M |
| | 9 or Last | V |
| HLA B*0702 | 2 | P |
| | 9 or Last | L, M, V, F |
| HLA B*35 | 2 | P |
| | 9 | Y, F, M |

In some embodiments, the immunogenicity of a target peptide is measured using transgenic mice expressing human MHC class I genes. For example, “ADD Tg mice” express an interspecies hybrid class I MHC gene, AAD, which contains the α-1 and α-2 domains of the human HLA-A2.1 gene and the α-3 transmembrane and cytoplasmic domains of the mouse H-2Dd gene, under the transcriptional control of the human HLA-A2.1 promoter. Immunodetection of the HLA-A2.1 recombinant transgene established that expression was at equivalent levels to endogenous mouse class I molecules. The mouse α-3 domain expression enhances the immune response in this system. Compared to unmodified HLA-A2.1, the chimeric HLA-A2.1/H2-Dd MHC Class I molecule mediates efficient positive selection of mouse T cells to provide a more complete T cell repertoire capable of recognizing peptides presented by HLA-A2.1 Class I molecules.

The peptide epitopes presented and recognized by mouse T cells in the context of the HLA-A2.1/H2-Dd class I molecule are the same as those presented in HLA-A2.1⁺ humans. This transgenic strain enables the modeling of human T cell immune responses to HLA-A2 presented antigens, and identification of those antigens. This transgenic strain is a preclinical model for design and testing of vaccines for infectious diseases or cancer therapy involving optimal stimulation of CD8⁺ cytolytic T cells.

In some embodiments, the immunogenicity of a modified O-GlcNAc peptide is determined by the degree of Interferon gamma (IFNγ) and/or tumor necrosis factor-alpha (TNF-α) production of T cells from ADD Tg mice immunized with the target peptide, e.g., by immunization with target peptide pulsed bone marrow derived dendritic cells.

In some embodiments, the modified target peptides are about or at least 10, 20, 30, 40, 50, 60, 70, 80, 90, 95, 100, 110, 125, 150, 175, 200, 225, 250, 275, 300, 350, 375, 400, 450, 500, 600, 700, 800, 1000, 1500, 2000, 2500, 3000, 4000, 5000% or more immunogenic, e.g., in terms of numbers of IFNγ- and/or TNF-α-positive (i.e., “activated”) T cells relative to numbers elicited by native target peptides in ADD Tg mice immunized with O-GlcNAc peptide-pulsed bone marrow derived dendritic cells (BMDCs). In some embodiments, the target peptides are modified target peptides. In some embodiments, the modified target peptides are able to elicit CD8⁺ T cells that are cross-reactive with the modified and the native target peptide in general and when such modified and native target peptides are complexed with MEW class I molecules in particular. In some embodiments, the CD8⁺ T cells that are cross-reactive with the modified and the native target peptides are able to reduce tumor size by about or at least 10%, 20%, 30%, 40%, 50%, 60%, 70%, 80%, 90%, 95%, 97% or 99% in a NOD/SCID/IL-2Rγc^{-/-} knock out mouse relative to IL-2 treatment without such cross-reactive CD8⁺ T cells.

The phrase “capable of inducing a target peptide-specific memory T cell response in a patient” as used herein relates to eliciting a response from memory T cells (also referred to as “antigen-experienced T cell”), which are a subset of infection- and cancer-fighting T cells that have previously encountered and responded to their cognate antigen. Such T cells can recognize foreign invaders, such as bacteria or viruses, as well as cancer cells. Memory T cells have become “experienced” by having encountered antigen during a prior infection, having encountered cancer, or via previous vaccination. At a second encounter with the cognate antigen (e.g., by way of an initial inoculation with a target peptide of the presently disclosed subject matter), memory T cells can reproduce to mount a faster and stronger immune response than the first time the immune system responded to the invader (e.g., through the body's own consciously unperceived recognition of a target peptide being associated with diseased tissue). This behavior can be assayed in T lymphocyte proliferation assays, which can reveal exposure to specific antigens.

Memory T cells comprise two subtypes: central memory T cells (T_{CM} cells) and effector memory T cells (T_{EM} cells). Memory cells can be either CD4⁺ or CD8⁺. Memory T cells typically express the cell surface protein CD45RO. Central memory (T_{CM}) cells generally express L-selectin and CCR7, and they secrete IL-2 but not IFNγ or IL-4. Effector memory (T_{EM}) cells, however, generally do not express L-selectin or CCR7 but produce effector cytokines like IFNγ and IL-4.

A memory T cell response generally results in the proliferation of memory T cells and/or the upregulation or increased secretion of factors such as CD45RO, L-selectin, CCR7, IL-2, IFNγ, CD45RA, CD27, and/or IL-4. In some embodiments, the target peptides of the presently disclosed subject matter are capable of inducing a T_{CM} cell response associated with L-selectin, CCR7, IL-2 but not IFNγ or IL-4 expression and/or secretion. See e.g., Hamann et al., 1997. In some embodiments, a T_{cm} cell response is associated with an at least or an about 1%, 5%, 10%, 15%, 20%, 25%, 30%, 35%, 45%, 50%, 55%, 60%, 65%, 70%, 75%, 80%, 90%, 95%, 97%, 98%, 99%, 100%, 125%, 150%, 175%, 200%, 250%, 300%, 400%, 500%, 600%, 700%, 800%, 900%, 1000%, 1500%, 2000% or more increase in T cell CD45RO/RA, L-selectin, CCR7, or IL-2 expression and/secretion.

In some embodiments, the target peptides of the presently disclosed subject matter are capable of inducing a CD8⁺ T_{CM} cell response in a patient the first time that patient is provided the composition including the selected target peptides. As such, the target peptides of the presently disclosed subject matter can in some embodiments be referred to as “neo-antigens.” Although target peptides might be considered “self” for being derived from self-tissue, they generally are only found on the surface of cells with a dysregulated metabolism (e.g., aberrant phosphorylation and/or glycosylation), and they are likely never presented to immature T cells in the thymus. As such, these “self” antigens act are neo-antigens because they are nevertheless capable of eliciting an immune response.

In some embodiments, about or at least 1%, 5%, 10%, 15%, 20%, 25%, 30%, 35%, 45%, 50%, 55%, 60%, 65%, 70%, 75%, 80%, 90%, 95%, 97%, 98%, or 99% of T cells activated by particular target peptide in a particular patient sample are T_{CM} cells.

In some embodiments, a patient sample is isolated exactly, about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days after an initial exposure to a particular target peptide and then assayed for target peptide-specific activated T cells and the proportion of T_{CM} cells thereof.

In some embodiments, the compositions of the presently disclosed subject matter are able to elicit a CD8⁺ T_{CM} cell response in at least or about 1%, 5%, 10%, 15%, 20%, 25%, 30%, 35%, 45%, 50%, 55%, 60%, 65%, 70%, 75%, 80%, 90%, 95%, 97%, 98%, or 99% of patients and/or healthy volunteers.

In some embodiments, the compositions of the presently disclosed subject matter are able to elicit a CD8⁺ T_{CM} cell response in about or at least 1%, 5%, 10%, 15%, 20%, 25%, 30%, 35%, 45%, 50%, 55%, 60%, 65%, 70%, 75%, 80%, 90%, 95%, 97%, 98%, 99% of patients and/or healthy volunteers specific, and in some embodiments the CD8⁺ T_{CM} cell response elicited is directed against all or at least or about 1, 2, 3, 4, 5, 6, 7, 8, 9, 10, or more target peptides that are present in the composition. In some embodiments, the aforementioned T cell activation tests are done by ELISpot assay.

The term “O-GlcNAc peptides” thus includes MHC class I-specific O-GlcNAc peptides. Exemplary MHC class I target peptides set forth in Tables 3-7, herein.

Modification of proteins with O-linked β-N-acetylglucosamine (O-GlcNAc) was previously technically difficult to detect. However, it rivals phosphorylation in both abundance and distribution of the protein targets for this modification. Like phosphorylation, O-GlcNAcylation is a reversible modification of nuclear and cytoplasmic proteins and is characterized by the attachment of a single β-N-acetyl-glucosamine moiety to a hydroxyl group of a serine or a threonine residue. Modification by O-GlcNAcylation is often competitive with phosphorylation at the same sites or at proximal sites on proteins. Furthermore, crosstalk between O-GlcNAcylation and phosphorylation affects the posttranslational state of hundreds of proteins in response to nutrients and stress, and plays an important role in chronic diseases of metabolism such as but not limited to diabetes and neurodegeneration.

O-GlcNAc transferase (OGT) catalyzes the addition of the sugar moiety from the donor substrate uridine 5′-diphosphate (UDP)-GlcNAc to proteins. During M phase, OGT localizes to discrete structures, such as centrosomes (metaphase) and the spindle (anaphase), and then moves to the midbody during cytokinesis. OGT along with O-GlcNAcase (OGA), the enzyme that removes the sugar, dynamically interact with Aurora Kinase B (AURKB) and protein phosphatase 1 (PP1) at the midbody. Together, these proteins form a complex regulating M-phase O-GlcNAcylation, which in turn influences the phosphorylation state, of vimentin. However, the identity of other OGT mitotic substrates is currently not known.

Peptides modified with O-GlcNAc and/or hexose-O-GlcNAc disaccharides can be difficult to detect by standard mass spectrometric methods. The modification is usually present at sub-stoichiometric amounts, modified and unmodified peptides co-elute during high-performance liquid chromatography (HPLC), and ionization of the modified peptide is suppressed in the presence of unmodified peptides. Consequently, sample enrichment is often required to successfully detect and characterize O-GlcNAcylated peptides. Enrichment can be achieved through chemoenzymatic approaches that biotinylate O-GlcNAc peptides and capture them by avidin chromatography.

Alternatively, a chemoenzymatic approach using a photocleavable biotin-alkyne reagent (PCbiotin-alkyne) tag can be used. See e.g., Figure S1A of Wang et al., 2010b (“Wang”), herein incorporated by reference. Photocleavage not only allows efficient and quantitative recovery from the affinity column, but also tags the peptide with a charged moiety that facilitates O-GlcNAc site mapping by electron-transfer dissociation (ETD) mass spectrometry. This tagging approach also makes it possible to use conventional collision-activated dissociation mass spectrometry (CAD MS) to screen samples for the presence of O-GlcNAc-modified peptides by monitoring for two-signature fragment ions characteristic of the tag (see Figure S1B of Wang).

O-GlcNAcylation rivals phosphorylation in both abundance and distribution of the modified proteins and alterations in O-GlcNAcylation disrupt both the chromosomal passenger complex containing AURKB, inner centromere protein antigens 135/155 kDa (INCENP), PP1, Borealin, and Survivin—and the circuits regulating CDK1 activity.

O-GlcNAc moieties are nearly as abundant as phosphates on proteins associated with the spindle and midbody. Many of the O-GlcNAcylation sites identified are identical or proximal to known phosphorylation sites. O-GlcNAcylation and phosphorylation work together to control complicated mitotic processes, such as spindle formation. For example, OGT overexpression altered the abundance of transcripts and proteins encoded by several mitotic genes, changed the localization of NuMA1, and disrupted the chromosomal passenger complex and the CDK1 activation circuit.

An interplay exists between O-GlcNAcylation and phosphorylation for several protein classes, most noticeably transcriptional regulators and cytoskeletal proteins. Many of the O-GlcNAcylation and phosphorylation sites are located in the regulatory head domains of intermediate filament proteins. Phosphorylation of these sites causes filament disassociation during M phase. For example, vimentin is phosphorylated at multiple sites during M phase and there is an O-GlcNAcylation site that is also a mitotic phosphorylation site (Ser55; Slawson et al., 2005; Slawson et al., 2008; Wang et al., 2007; Molina et al., 2007). There three additional O-GlcNAcylation sites on vimentin at Ser7, Thr33, and Ser34 (see Tables S5 and S6 of Wang), all of which are in the regulatory head domain of the protein. Two of these, Ser7 and Ser34, are also phosphorylation sites (Dephoure et al., 2008; Molina et al., 2007). Signaling pathways involving cytoskeletal proteins are regulated by reciprocal occupancy on specific sites by phosphate and O-GlcNAc. In these classes of molecules, areas of multiple phosphorylations are also likely to be targeted for O-GlcNAcylation.

OGT overexpression profoundly affects multiple mitotic signaling circuits. Although overexpression of OGT does not interfere with the formation of the midbody complex or localization of AURKB, AURKB activity is altered toward the cytoskeletal protein, vimentin. The reduction in the abundance of AURKB or INCENP dampens kinase activity to a point that retards mitotic progression especially during anaphase and telephase. Furthermore, OGT overexpression reduced phosphorylation of INCENP and borealin, but to what extent this alters the function of the midbody complex is unclear.

Multiple components of the cyclin B/CDK1 activation circuit were disrupted by the overexpression of OGT. The loss of PLK1 inhibitory phosphorylation on MYT1 and the increase in the abundance of MYT1 are likely contributors to the loss in cyclin B-CDK1 activity observed in OGT-overexpressing cells (see FIG. 7 of Wang). However, the reduction in cyclin B-CDK1 activity is likely only partially due to the increase in MYT1 activity, because the mRNA for CDC25C, the key CDK1 dual-specific phosphatase, is substantially reduced. The “on” switch for CDK1 activation, the reduction of MYT1 and the increase in CDC25C activity, is pushed toward “off” by OGT overexpression. Both MYT1 and CDC25C are substrates for PLK1. The protein and transcript abundance of PLK1 is substantially reduced in response to OGT overexpression, but there is little change in the extent of activating phosphorylation of PLK1.

Because O-GlcNAcylation is directly coupled to nutrient uptake and metabolism, the sugar residue is an ideal metabolic sensor for regulating mitotic progression. Whereas, phosphorylation might act as a master switch initiating the mitotic process, O-GlcNAcylation could act as an adjuster of signals to make these processes more responsive to environmental cues. How O-GlcNAcylation exerts control on specific mitotic proteins and how O-GlcNAcylation will integrate into well-known signaling pathways represent another layer of cellular regulation.

II.B. Immunosuitability

Although individuals in the human population display hundreds of different HLA alleles, some are more prevalent than others. For example, 88% of melanoma patients carry at least one of the six HLA alleles: HLA-A*0201 (51%), HLA-A*0101 (29%), HLA-A*0301 (21%), HLA-B*4402 (27%), HLA-B*0702 (30%), and HLA-B*-2705 (7%).

The presently disclosed subject matter provides in some embodiments target peptides which are immunologically suitable for each of the foregoing HLA alleles. “Immunologically suitable” means that a target peptide will bind at least one allele of an MHC class I molecule in a given patient. Compositions of the presently disclosed subject matter are in some embodiments immunologically suitable for a patient when at least one target peptide of the composition will bind at least one allele of an MHC class I molecule in a given patient. Compositions of multiple target peptides presented by each of the most prevalent alleles used in a cocktail ensures coverage of the human population and to minimize the possibility that the tumor will be able to escape immune surveillance by down-regulating expression of any one class I target peptide.

The compositions of the presently disclosed subject matter can in some embodiments comprise at least one target peptide specific for one or more of the following alleles: HLA-A*0201, HLA-B*0702, and HLA-B*35. The compositions of the presently disclosed subject matter can in some embodiments have at least one target peptide specific for one or more of the following alleles HLA-A*0201, HLA-B*0702, and HLA-B*35. Alternatively, the compositions of the presently disclosed subject matter can in some embodiments have at least one target peptide specific for HLA-A*0201, HLA-B*0702, and HLA-B*35, or any combination thereof. The compositions may have at least one O-GlcNAc peptide specific for about or at least 1, 2, or all 3 of the aforementioned alleles.

As such, the compositions of the presently disclosed subject matter containing various combinations of target peptides are in some embodiments immunologically suitable for between or about 3-88%, 80-89%, 70-79%, 60-69%, 57-59%, 55-57%, 53-55% or 51-53% or 5-90%, 10-80%, 15-75%, 20-70%, 25-65%, 30-60%, 35-55% or 40-50% of the population of a particular cancer including, but not limited to a leukemia. In some embodiments, the compositions of the presently disclosed subject matter are able to act as vaccine compositions for eliciting anti-tumor immune responses and/or in adoptive T cell therapy of leukemia patients wherein the compositions are immunologically suitable for about or at least 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 20, 19, 18, 17, 16, 15, 14, 13, 12, 11, 10, 9, 8, 7, 6, 5, 4, or 3 percent of cancer such as, but not limited to leukemia patients.

### III. Compositions

The phrase “target peptide compositions” as used herein refers to at least one target peptide formulated, for example, as a vaccine; or as a preparation for pulsing cells in a manner such that the pulsed cells, e.g., dendritic cells, will display the at least one target peptide in the composition on their surface, e.g., to T cells in the context of adoptive T cell therapy.

The compositions of the presently disclosed subject matter can in some embodiments include about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-65, 65-80, 80-120, 90-150, 100-175, or 175-250 different target peptides.

The compositions of the presently disclosed subject matter in some embodiments generally include WIC class I specific target peptide(s), but can also include one or more target peptides associated with tumors (e.g., tumor associated antigen (“TAA”)) such as, but not limited to those disclosed in Table 2.

TABLE 2
Exemplary Tumor-associated Antigen Peptides

| Tumor-associated Antigen¹ | Peptide Sequence |
|---|---|
| CAMEL₁₉₋₂₇ | AAQERRVPR (SEQ ID NO: 46) |
| CEA₂₇₋₃₅ | HLFGYSWYK (SEQ ID NO: 47) |
| CEA₅₇₁₋₅₇₉ | YLSGADLNL (SEQ ID NO: 48) |
| DCT₁₉₇₋₂₀₅ | LLGPGRPYR (SEQ ID NO: 49) |
| FBP₁₉₁₋₁₉₉ | EIWTHSYKV (SEQ ID NO: 50) |
| gp100₁₇₋₂₅ | ALLAVGATK (SEQ ID NO: 51) |
| gp100₄₄₋₅₉ | WNRQLYPEWTEAQRLD (SEQ ID NO: 52) |
| gp100₈₇₋₉₅ | ALNFPGSQK (SEQ ID NO: 53) |
| gp100₁₅₄₋₁₆₂ | KTWGQYWQV (SEQ ID NO: 54) |
| gp100₂₀₉₋₂₁₇ | ITDQVPFSV (SEQ ID NO: 55) |
| gp100₂₀₉₋₂₁₇ | IMDQVPFSV (SEQ ID NO: 56) |
| gp100₂₈₀₋₂₈₈ | YLEPGPVTA (SEQ ID NO: 57) |
| gp100₄₇₆₋₄₈₅ | VLYRYGSFSV (SEQ ID NO: 58) |
| gp100₆₁₄₋₆₂₂ | LIYRRRLMK (SEQ ID NO: 59) |
| Her2/neu₃₆₉₋₃₇₇ | KIFGSLAFL (SEQ ID NO: 60) |
| Her2/neu₇₅₄₋₇₆₂ | VLRENTSPK (SEQ ID NO: 61) |
| MAGE-A1, 2, 3, 6₁₂₁₋₁₃₄ | LLKYRAREPVTKAE (SEQ ID NO: 62) |
| MAGE-A1₁₆₁₋₁₆₉ | EADPTGHSY (SEQ ID NO: 63) |
| MAGE-A1₉₆₋₁₀₄ | SLFRAVITK (SEQ ID NO: 64) |
| MAGE-A3₁₆₈₋₁₇₆ | EVDPIGHLY (SEQ ID NO: 65) |
| MAGE-A3₂₈₁₋₂₉₅ | TSYVKVLHEIMVKISG (SEQ ID NO: 66) |
| MAGE-A10₂₅₄₋₂₆₂ | GLYDGMEHL (SEQ ID NO: 67) |
| MART-1₂₇₋₃₅ | AAGIGILTV (SEQ ID NO: 68) |
| MART-1₅₁₋₇₃ | RNGYRALMDKSLHVGTQCALTRR (SEQ ID NO: 69) |
| MART-1₉₇₋₁₁₆ | VPNAPPAYEKLsAEQSPPPY (SEQ ID NO: 70) |
| MART-1₉₈₋₁₀₉ | PNAPPAYEKLsA (SEQ ID NO: 71) |
| MART-1₉₉₋₁₁₀ | NAPPAYEKLsAE (SEQ ID NO: 72) |
| MART-1₁₀₀₋₁₀₈ | APPAYEKLs (SEQ ID NO: 73) |
| MART-1₁₀₀₋₁₁₁ | APPAYEKLsAEQ (SEQ ID NO: 74) |
| MART-1₁₀₀₋₁₁₄ | APPAYEKLsAEQSPP (SEQ ID NO: 75) |
| MART-1₁₀₀₋₁₁₅ | APPAYEKLsAEQSPPP (SEQ ID NO: 76) |
| MART-1₁₀₀₋₁₁₆ | APPAYEKLsAEQSPPPY (SEQ ID NO: 77) |
| MART-1₁₀₁₋₁₀₉ | PPAYEKLsA (SEQ ID NO: 78) |
| MART-1₁₀₁₋₁₁₂ | PPAYEKLsAEQS (SEQ ID NO: 79) |
| MART-1₁₀₂₋₁₁₀ | PAYEKLsAE (SEQ ID NO: 80) |
| MART-1₁₀₂₋₁₁₃ | PAYEKLsAEQSP (SEQ ID NO: 81) |
| MART-1₁₀₃₋₁₁₄ | AYEKLsAEQSPP (SEQ ID NO: 82) |
| MART-1₁₀₄₋₁₁₅ | YEKLsAEQSPPP (SEQ ID NO: 83) |
| NY-ESO-1₅₃₋₆₂ | ASGPGGGAPR (SEQ ID NO: 84) |
| p2₈₃₀₋₈₄₄ | AQYIKANSKFIGITEL (SEQ ID NO: 85) |
| TAG-1, 2 | RLSNRLLLR (SEQ ID NO: 86) |
| Tyr₅₆₋₇₀ | AQNILLSNAPLGPQFP (SEQ ID NO: 87) |
| Tyr₁₄₆₋₁₅₆ | SSDYVIPIGTY (SEQ ID NO: 88) |
| Tyr₂₄₀₋₂₅₁ | SDAEKSDICTDEY (SEQ ID NO: 89) |
| Tyr₂₄₃₋₂₅₁ | KCDICTDEY (SEQ ID NO: 90) |
| Tyr₃₆₉₋₃₇₇ | DYMDGTMSQV (SEQ ID NO: 91) |
| Tyr₃₈₈₋₄₀₆ | FLLHHAFVDSIFEQWLQRHRP (SEQ ID NO: 92) |

¹the numbers listed in lowercase denote the amino acid positions of the peptide
sequences for each TAA; a lowercase “s” in the amino acid sequences indicate
that the corresponding serine residue can in some embodiments be phosphorylated.

Compositions comprising the O-GlcNAc peptides of the presently disclosed subject matter are typically substantially free of other human proteins or peptides. They can be made synthetically or by purification from a biological source. They can be made recombinantly. Desirably they are in some embodiments at least 90% pure, in some embodiments at least 92% pure, in some embodiments at least 93% pure, in some embodiments at least 94% pure, in some embodiments at least 95% pure, in some embodiments at least 96% pure, in some embodiments at least 97% pure, in some embodiments at least 98% pure, and in some embodiments at least 99% pure. For administration to a human, they generally do not contain other components that might be harmful to a human recipient (referred to herein as “pharmaceutically acceptable for use in a human”). The compositions are typically devoid of cells, both human and recombinant producing cells. However, as noted below, in some cases, it can be desirable to load dendritic cells with a target peptide and use those loaded dendritic cells as either an immunotherapy agent themselves or as a reagent to stimulate a patient's T cells ex vivo. The stimulated T cells can be used as an immunotherapy agent.

In some cases, it can be desirable to form a complex between a target peptide and an HLA molecule of the appropriate type. Such complexes can be formed in vitro or in vivo. Such complexes are in some embodiments tetrameric with respect to an HLA-target peptide complex.

Under certain circumstances it can be desirable to add additional proteins or peptides, for example, to make a cocktail having the ability to stimulate an immune response in a number of different HLA type hosts. Alternatively, additional proteins and/or peptides can provide an interacting function within a single host, such as but not limited to an adjuvant function or a stabilizing function. As a non-limiting example, other tumor antigens can be used in admixture with the target peptides such that multiple different immune responses are induced in a single patient.

Administration of target peptides to a mammalian recipient can be accomplished using long target peptides (e.g., longer than 15 residues, which can be in some embodiments up to 20 residues, in some embodiments up to 30 residues, in some embodiments up to 40 residues, in some embodiments up to 50 residues, and in some embodiments longer than 50 residues), and/or using target peptide-loaded dendritic cells. See Melief, 2009. In some embodiments, an immediate goal of the administration of target peptides is to induce activation of CD8⁺ T cells in a subject. Additional components that can be administered to the same subject, either at the same time and/or close in time (such as but not limited to within 3, 5, 7, 10, 14, 17, or 21 days of each other, or even longer) include TLR-ligand oligonucleotide CpG and related target peptides that have overlapping sequences of at least six amino acid residues. To ensure efficacy, mammalian recipients should express the appropriate human HLA molecules to bind to the target peptides. Transgenic mammals can be used as recipients, for example, if they express appropriate human HLA molecules. If a mammal's own immune system recognizes a similar target peptide then it can be used as model system directly without introducing a transgene. Useful models and recipients can be at increased risk of developing cancer, such as but not limited to leukemia. Other useful models and recipients can be predisposed, e.g., genetically and/or environmentally, to develop leukemia or another cancer.

IIIA. Selection of Target Peptides

Disclosed herein is the finding that immune responses can be generated against O-GlcNAcylated peptides tested in healthy and diseased individuals. The T cells associated with these immune responses, when expanded in vitro, are able to recognize and kill malignant tissue (both established cells lines and primary tumor samples). Cold-target inhibition studies reveal that these target peptide-specific T cell lines kill primary tumor tissue in a target peptide-specific manner.

When selecting target peptides of the presently disclosed subject matter for inclusion in immunotherapy, e.g., in adaptive cell therapy or in the context of a vaccine, one can in some embodiments pick target peptides using one or more of the following criteria: 1) peptides associated with a particular cancer/tumor cell type; 2) a peptide derived from a gene product (e.g., a polypeptide) associated with cell proliferation, transformation, and/or malignancy; 3) a peptide that is specific for an HLA allele carried the group of patients to be treated; and/or 4) a peptide that is capable of inducing a target peptide-specific memory T cell response in the patients to be treated upon a first exposure to a composition including the selected target peptides.

III.B. Target Peptide Vaccines

The antigen target peptides can also be employed in a composition designed to vaccinate an individual. The antigen target peptides can in some embodiments be injected alone and can in some embodiments be administered in combination with an adjuvant and/or a pharmaceutically acceptable carrier. Vaccines are envisioned to prevent and/or treat certain diseases in general, and cancers in particular.

The target peptide-containing compositions of the presently disclosed subject matter can in some embodiments be used as a vaccine for cancer, and more specifically for leukemia, melanoma, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer. The compositions can include target peptides. The vaccine compositions can in some embodiments include only the target peptides, or peptides disclosed herein, or they can include other cancer antigens that have been identified.

The vaccine compositions of the presently disclosed subject matter can be used prophylactically for the purposes of preventing, reducing the risk of, and/or delaying initiation of a cancer in an individual that does not currently have cancer. Alternatively, they can be used to treat an individual that already has cancer, so that recurrence or metastasis is delayed and/or prevented. Prevention relates to a process of prophylaxis in which the individual is immunized prior to the induction or onset of cancer. For example, in some embodiments individuals with a history of severe sunburn and at risk for developing melanoma can be immunized prior to the onset of the disease.

Alternatively, individuals that already have cancer can be immunized with the target peptide-containing compositions of the presently disclosed subject matter so as to stimulate an immune response that would be reactive against the cancer. A clinically relevant immune response would be one in which the cancer partially or completely regresses and is eliminated from the patient, and it would also include those responses in which the progression of the cancer is blocked without being eliminated. Similarly, prevention need not be total, but may result in a reduced risk, delayed onset, or delayed progression or metastasis.

In some embodiments, the vaccines of the presently disclosed subject matter can be used to treat leukemia. The target peptide vaccines of the presently disclosed subject matter can in some embodiments be given to patients before, after, or during any stages of leukemia.

In some embodiments, the 5-year survival rate of patients treated with the vaccines of the presently disclosed subject matter is increased by a statistically significant amount: e.g., by about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or even greater than 100 percent, relative to the average 5-year survival rates described above.

In some embodiments, the target peptide vaccine compositions of the presently disclosed subject matter increase survival rates in patients with leukemia by a statistically significant amount of time such as, but not limited to by about or at least 0.25, 0.5, 0.75, 1.0, 1.25, 1.5, 1.75, 2.0, 2.25, 2.5, 2.75, 3.0, 3.25, 3.5, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.50, 9.75, 10.0, 10.25, 10.5, 10.75, 11.0, 11.25, 11.5, 11.75, or 12 months or more compared to what could have been expected without vaccine treatment at the time of filing of this specification.

In some embodiments, the survival rate (e.g., the 1, 2, 3, 4, or 5-year survival rate) of patients treated with the vaccines of the presently disclosed subject matter is increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent, or even greater than 100 percent, relative to the average 5-year survival rates described above.

The target peptide vaccines of the presently disclosed subject matter are in some embodiments envisioned to illicit a T cell-associated immune response such as, but not limited to generating activated CD8⁺ T cells specific for native target peptide/MHC class I expressing cells. In some embodiments, the CD8⁺ T cells specific for native target peptide/MHC class I expressing cells are specific for at least or about 1, 2, 3, 4, 5, 6, 7, 8, 9, 10, or more of the target peptides in the vaccine in a patient for about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more days after providing the vaccine to the patient.

In some embodiments, the treatment response rates of patients treated with the target peptide vaccines of the presently disclosed subject matter are increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 07, 98, 99, 100, 150, 200, 250, 300, 350, 400, 450, or 500 or more percent, relative to treatment without the vaccine.

In some embodiments, overall median survival of patients treated with the target peptide vaccines of the presently disclosed subject matter is increased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, or 500 or more percent, relative to treatment without the vaccine. In some embodiments, the overall median survival of leukemia patients treated the target peptide vaccines is envisioned to be about or at least 10.0, 10.25, 10.5, 10.75, 11.0, 11.25, 11.5, 11.75, 12, 12.25, 12.5, 12.75, 13, 13.25, 13.5, 13.75, 14, 14.25, 14.5, 14.75, 15, 16, 17, 18, 19, 20, 21, 22, 23, 24, 25, 26, 27, 28, 29, 30, 31, 32, 33, 34, 35, 36 or more months.

In some embodiments, tumor size of patients treated with the target peptide vaccines of the presently disclosed subject matter is decreased by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, or 500 or more percent, relative to treatment without the vaccine.

In some embodiments, the compositions of the presently disclosed subject matter provide a clinical tumor regression that is by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of patients treated with the composition.

In some embodiments, the compositions of the presently disclosed subject matter provide a CTL response specific for the cancer being treated, e.g., leukemia, by a statistically significant amount such as, but not limited to about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of patients treated with the composition.

In some embodiments, the compositions of the presently disclosed subject matter provide an increase in progression free survival in the cancer being treated, such as but not limited to leukemia, of about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500 or more months compared to the progression free survival or patients not treated with the composition.

In some embodiments, one or more of progression free survival, CTL response rates, clinical tumor regression rates, tumor size, survival rates (such as but not limited to overall survival rates), and/or response rates are determined, assessed, calculated, and/or estimated weekly, monthly, bi-monthly, quarterly, semi-annually, annually, and/or bi-annually over a period of about or at least 1, 2, 3, 4, 5, 6, 7, 8, 9, 10, 11, 12, 13, 14, 15 or more years or about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 225, 250, 275, 300, 325, 350, 375, 400, 425, 450, 475, 500 or more weeks.

III.C. Compositions for Priming T Cells

Adoptive cell transfer (ACT) is the passive transfer of cells, in some embodiments immune-derived cells, into a recipient host with the goal of transferring the immunologic functionality and characteristics into the host. Clinically, this approach has been exploited to transfer either immune-promoting or tolerogenic cells (often lymphocytes) to patients to enhance immunity against cancer. The adoptive transfer of autologous tumor infiltrating lymphocytes (TIL) or genetically redirected peripheral blood mononuclear cells has been used to successfully treat patients with advanced solid tumors, including melanoma and colorectal carcinoma, as well as patients with CD19-expressing hematologic malignancies. In some embodiments, ACT therapies achieve T cell stimulation ex vivo by activating and expanding autologous tumor-reactive T cell populations to large numbers of cells that are then transferred back to the patient. See Gattinoni et al., 2006.

The target peptides of the presently disclosed subject matter can in some embodiments take the form of antigenic peptides formulated in a composition added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigenic target peptides.

Alternatively, the target peptides can be added to dendritic cells (DCs) in vitro to produce loaded DCs, with the loaded DCs being subsequently transferred into an individual with cancer in order to stimulate an immune response. Alternatively, the loaded DCs can be used to stimulate CD8⁺ T cells ex vivo with subsequent reintroduction of the stimulated T cells to the patient. Although a particular target peptide might be identified on one particular cancer cell type, it might also be found on other cancer cell types.

The presently disclosed subject matter envisions treating cancer by providing a patient with cells pulsed with a composition of target peptides. The use of DCs pulsed with target peptides peptide antigens enables manipulation of the immunogen in two ways: varying the number of cells injected and varying the density of antigen presented on each cell. Exemplary non-limiting methods for DC-based based treatments can be found, for example in Mackensen et al., 2000.

III.D. Additional Peptides Present in Target Peptide Compositions

The target peptide compositions (or target peptide composition kits comprising the same) of the presently disclosed subject matter can in some embodiments also include at least one additional peptide derived from one or more tumor-associated antigens (TAAs). Examples of TAAs include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, prostatic acid phosphatase, and the like. Exemplary, non-limiting peptides derived from TAAs that can be incorporated into target peptide compositions (or target peptide composition kits comprising the same) of the presently disclosed subject matter are presented in Table 2 above.

Such tumor-specific peptides can be added to the target peptide compositions in a manner, number, and in an amount as if they were an additional target peptide added to the target peptide compositions as described herein.

In some embodiments, the tumor-specific peptides added to the target peptide compositions of the presently disclosed subject matter comprise, consist essentially of, or consist of the amino acid sequences ALLAVGATK (SEQ ID NO: 51); IMDQVPFSV (SEQ ID NO: 56); YLEPGPVTA (SEQ ID NO: 57); LIYRRRLMK (SEQ ID NO: 59); EADPTGHSY (SEQ ID NO: 63); SLFRAVITK (SEQ ID NO: 64); EVDPIGHLY (SEQ ID NO: 65); GLYDGMEHL (SEQ ID NO: 67); ASGPGGGAPR (SEQ ID NO: 84); SSDYVIPIGTY (SEQ ID NO: 88); SDAEKSDICTDEY (SEQ ID NO: 89); YMDGTMSQV (SEQ ID NO: 91), or any combinations thereof.

III.E. Combination Therapies

In some embodiments, the target peptide compositions (or target peptide composition kits) of the presently disclosed subject matter are administered as a vaccine or in the form of pulsed cells as first, second, third, or fourth line treatment for the cancer. In some embodiments, the compositions of the presently disclosed subject matter are administered to a patient in combination with one or more therapeutic agents. Exemplary, non-limiting therapeutic agents include anti-Programed Death-1 (PD1) or PD1-antagonists such as the anti-PD1 antibody BMS-936558 (Bristol-Myers Squibb Co., New York, N.Y., United States of America); anti-CTLA-4 or CTLA-4 antagonists; vermurafenib; ipilimumab; Dacarbazine; IL-2; Temozolomide; receptor tyrosine kinase inhibitors, including but not limited to imatinib, gefitinib, erlotinib, sunitinib, tyrphostins, telatinib; sipileucel-T; a platinum-based agent; a taxane; an alkylating agent; an antimetabolite and/or a vinca alkaloid; and combinations thereof.

In some embodiments, the cancer is sensitive to and/or refractory, relapsed, and/or resistant to one or more chemotherapeutic agents such as, but not limited to a platinum-based agent, a taxane, an alkylating agent, an anthracycline (e.g., doxorubicin including but not limited to liposomal doxorubicin), an antimetabolite, and/or a vinca alkaloid. In some embodiments, the cancer is an ovarian cancer, and the ovarian cancer is refractory, relapsed, or resistant to a platinum-based agent (e.g., carboplatin, cisplatin, oxaliplatin), a taxane (e.g., paclitaxel, docetaxel, larotaxel, cabazitaxel), and/or an anthracycline (e.g., doxorubicin including but not limited to liposomal doxorubicin). In some embodiments, the cancer is colorectal cancer, and the cancer is refractory, relapsed, or resistant to an antimetabolite (e.g., an antifolate (e.g., pemetrexed, floxuridine, raltitrexed) a pyrimidine analogue (e.g., capecitabine, cytrarabine, gemcitabine, 5FU)), and/or a platinum-based agent (e.g., carboplatin, cisplatin, oxaliplatin). In some embodiments, the cancer is lung cancer, and the cancer is refractory, relapsed, or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is breast cancer, and the cancer is refractory, relapsed, or resistant to a taxane (e.g., paclitaxel, docetaxel, larotaxel, cabazitaxel), a VEGF pathway inhibitor, an anthracycline (e.g., daunorubicin, doxorubicin including but not limited to liposomal doxorubicin, epirubicin, valrubicin, idarubicin), a platinum-based agent (e.g., carboplatin, cisplatin, oxaliplatin), and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is gastric cancer, and the cancer is refractory, relapsed, or resistant to an antimetabolite (e.g., an antifolate including but not limited to pemetrexed, floxuridine, raltitrexed) and a pyrimidine analogue (e.g., capecitabine, cytrarabine, gemcitabine, 5FU) and/or a platinum-based agent (e.g., carboplatin, cisplatin, oxaliplatin).

In some embodiments, the target peptide compositions (or target peptide composition kits) of the presently disclosed subject matter are associated with agents that inhibit T cell apoptosis or anergy thus potentiating a T cell response (referred to herein as a “T cell potentiator”). Such agents include B7RP1 agonists, B7-H3 antagonists, B7-H4 antagonists, HVEM antagonists, HVEM antagonists, GALS antagonists or alternatively CD27 agonists, OX40 agonists, CD137 agonists, BTLA agonists, ICOS agonists CD28 agonists, or soluble versions of PDL1, PDL2, CD80, CD96, B7RP1, CD137L, OX40 or CD70. See Pardoll, 2012.

In some embodiments, the T cell potentiator is a PD1 antagonist. Programmed death 1 (PD1) is a key immune checkpoint receptor expressed by activated T cells, and it mediates immunosuppression. PD1 functions primarily in peripheral tissues, where T cells can encounter the immunosuppressive PD1 ligands PD-L1 (B7-H1) and PD-L2 (B7-DC), which are expressed by tumor cells, stromal cells, or both. In some embodiments, the anti-PD1 monoclonal antibody BMS-936558 (also known as MDX-1106 and ONO-4538; Bristol-Myers Squibb) is used. In some embodiments, the T cell potentiator (e.g., PD1 antagonist) is administered as an intravenous infusion at least or about every 1, 1.5, 2, 2.5, 3, 3.5, or 4 weeks of each 4, 5, 6, 7, 8, 9, or 10-week treatment cycle of about for at least 1, 2, 3, 4, 5, 6, 7, 8, 9, 10, 11, 12, 13, 14, 15, 16, 17, 18, 19, 20, or more cycles. Exemplary, non-limiting doses of the PD1 antagonists are in some embodiments exactly, about, or at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, or more mg/kg. See Brahmer et al., 2012.

The exemplary therapeutic agents listed herein above are envisioned to be administered at a concentration of in some embodiments about 1 to 100 mg/m², in some embodiments about 10 to 80 mg/m², and in some embodiments about 40 to 60 mg/m². Further exemplary dosages include, but are not limited to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more mg/m². Alternatively, an exemplary dosage range can be in some embodiments about or at least 0.001 to 100 mg/kg, in some embodiments about or at least 0.1 to 1 mg/kg, and in some embodiments about or at least 0.01 to 10 mg/kg.

The target peptide compositions (or target peptide composition kits) of the presently disclosed subject matter can in some embodiments be co-administered with cytokines such as lymphokines, monokines, growth factors, and traditional polypeptide hormones. Exemplary cytokines are growth hormones including but not limited to human growth hormone, N-methionyl human growth hormone, and bovine growth hormone; parathyroid hormone; thyroxine; insulin; proinsulin; relaxin; prorelaxin; glycoprotein hormones including but not limited to follicle stimulating hormone (FSH), thyroid stimulating hormone (TSH), and luteinizing hormone (LH); hepatic growth factor; prostaglandin, fibroblast growth factor; prolactin; placental lactogen, OB protein; TNF-α and TNF-β; mullerian-inhibiting substance; mouse gonadotropin-associated peptide; inhibin; activin; VEGF; integrin; thrombopoietin (TPO); nerve growth factors including but not limited to NGF-β; platelet-growth factor; transforming growth factors (TGFs) including but not limited to TGF-α and TGF-β; insulin-like growth factor (IGF)-I and IGF-II; erythropoietin (EPO); osteoinductive factors; interferons (IFN) including but not limited to IFNα, IFNβ, and IFNγ; colony stimulating factors (CSFs) including but not limited to macrophage-CSF (M-CSF), granulocyte-macrophage-CSF (GM-CSF), and granulocyte-CSF (G-CSF); interleukins (ILs) including but not limited to IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, and IL-18; leukemia inhibitory factor (LIF), kit-ligand; FLT-3; angiostatin; thrombospondin; endostatin; and lymphotoxin (LT). As used herein, the term cytokine includes proteins from natural sources and/or from recombinant cell culture and biologically active equivalents thereof.

The target peptide compositions of the presently disclosed subject matter can in some embodiments be provided with administration of cytokines around the time of (including but not limited to about or at least 1, 2, 3, or 4 weeks or days before and/or after) the initial dose of a target peptide composition.

Exemplary non-limiting doses of the cytokine are in some embodiments about or at least 1-100, 10-80, 20-70, 30-60, 40-50, or 1, 2, 3, 4, 5, 6, 7, 8, 9 or 10 Mu/m²/day over about or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. The cytokine can in some embodiments be delivered at least or about once every 1, 2, 3, 4, 5, 6, 7, 8, 9, 10, 11, 12, 13, 14, 15, 16, 17, 18, 19, 20, 21, 22, 23, or 24 hours. Cytokine treatment can be provided in at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cycles of at least or about 1, 2, 3, 4, 5, 6, 7, 8, 9, 10, or more weeks, wherein each cycle has at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more cytokine doses. Cytokine treatment can in some embodiments be on the same schedule as administration of the target peptide compositions or in some embodiments on a different schedule, which differing schedule can in some embodiments be an overlapping schedule.

In some embodiments, the cytokine is IL-2 and is dosed in an amount about or at least 100,000 to 1,000,000; 200,000-900,000; 300,000-800,000; 450,000-750,000; 600,000-800,000; or 700,000-800,000 (in some embodiments. 720,000) units (IU)/kg administered, e.g., as a bolus, every 1, 2, 3, 4, 5, 6, 7, 8, 9, 10, 11, 12, 13, 14, 15, 16, 17, 18, 19, or 20 hours for 1, 2, 3, 4, 5, 6, 7, 8, 9, 10, 11, 12, 13, 14, or more days, in a cycle, for example.

### IV. Types of Proliferative Disease

In some embodiments, the compositions of the presently disclosed subject matter are envisioned to be useful in the treatment of benign and/or malignant proliferative diseases. Excessive proliferation of cells and turnover of cellular matrix contribute significantly to the pathogenesis of several diseases including but not limited to cancer, atherosclerosis, rheumatoid arthritis, psoriasis, idiopathic pulmonary fibrosis, scleroderma and cirrhosis of the liver, ductal hyperplasia, lobular hyperplasia, papillomas, and others.

In some embodiments, the proliferative disease is cancer, including but not limited to breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer. In some embodiments, the presently disclosed compositions and methods are used to treat acute myelogenous leukemia (AML), acute lyphocytic leukemia (ALL), chronic lymphocytic lymphoma (CLL), chronic myelogenous leukemia (CML), breast cancer, renal cancer, pancreatic cancer, and/or ovarian cancer.

In some embodiments, the cancer is a cancer described herein. For example, the cancer can be a cancer of the bladder (including but not limited to accelerated and metastatic bladder cancer), breast (including but not limited to estrogen receptor positive breast cancer, estrogen receptor negative breast cancer, HER-2 positive breast cancer, HER-2 negative breast cancer, triple negative breast cancer, and inflammatory breast cancer), colon (including but not limited to colorectal cancer), kidney (including but not limited to renal cell carcinoma), liver, lung (including but not limited to small cell lung cancer and non-small cell lung cancer such as but not limited to adenocarcinoma, squamous cell carcinoma, bronchoalveolar carcinoma and large cell carcinoma), genitourinary tract cancer, including but not limited to ovary (such as but not limited to fallopian, endometrial, and peritoneal cancers), cervix, prostate, and testes, lymphatic system, rectum, larynx, pancreas (including but not limited to exocrine pancreatic carcinoma), stomach (including but not limited to gastroesophageal, upper gastric, and lower gastric cancers), gastrointestinal cancer (including but not limited to anal cancer), gall bladder, thyroid, lymphoma (including but not limited to Burkitt's, Hodgkin's, and non-Hodgkin's lymphoma), leukemia (including but not limited to acute myeloid leukemia), Ewing's sarcoma, nasoesophageal cancer, nasopharyngeal cancer, neural and glial cell cancers (including but not limited to glioblastoma multiforme), and head and neck cancers. Exemplary non-limiting cancers also include melanoma, breast cancer (including but not limited to metastatic or locally advanced breast cancer), prostate cancer (including but not limited to hormone refractory prostate cancer), renal cell carcinoma, lung cancer (including but not limited to small cell lung cancer and non-small cell lung cancer (including adenocarcinoma, squamous cell carcinoma, bronchoalveolar carcinoma, and large cell carcinoma), pancreatic cancer, gastric cancer (including but not limited to gastroesophageal, upper gastric, and/or lower gastric cancer), colorectal cancer, squamous cell cancer of the head and neck, ovarian cancer (including but not limited to advanced ovarian cancer, platinum-based agent-resistant, and/or relapsed ovarian cancer), lymphoma (including but not limited to Burkitt's, Hodgkin's, or non-Hodgkin's lymphoma), leukemia (including but not limited to acute myelogenous leukemia (AML), acute lyphocytic leukemia (ALL), chronic lymphocytic lymphoma (CLL), and chronic myelogenous leukemia (CML)), and gastrointestinal cancer.

### V. Administration of Vaccine Compositions

V.A. Routes of Administration

The target peptide compositions of the presently disclosed subject matter can be administered parenterally, systemically, topically, or any combination thereof. By way of example and not limitation, composition injections can be performed by intravenous (i.v.) injection, subcutaneous (s.c.) injection, intradermal (i.d.) injection, intraperitoneal (i.p.) injection, and/or intramuscular (i.m.) injection. One or more such routes can be employed. Parenteral administration can be, for example, by bolus injection or by gradual perfusion over time. Alternatively or in addition, administration can be by the oral route.

In some embodiments, an injection is an intradermal (i.d.) injection. The target peptide compositions are in some embodiments suitable for administration of the peptides by any acceptable route such as but not limited to oral (enteral), nasal, ophthal, and transdermal. In some embodiments, the administration is subcutaneous, and in some embodiments the subcutaneous administration is by an infusion pump.

V.B. Formulations

Pharmaceutical carriers, diluents, and excipients are generally added to the target peptide compositions or (target peptide compositions kits) that are compatible with the active ingredients and acceptable for pharmaceutical use. Examples of such carriers include but are not limited to water, saline solutions, dextrose, and/or glycerol. Combinations of carriers can also be used.

The vaccine compositions of the presently disclosed subject matter can further incorporate additional substances to stabilize pH and/or to function as adjuvants, wetting agents, and/or emulsifying agents, which can serve to improve the effectiveness of the vaccine.

The target peptide compositions may include one or more adjuvants such as for example: montanide ISA-51 (Seppic Inc., Fairfield, N.J., United States of America); QS-21 (Aquila Biopharmaceuticals, Inc., Framingham, Mass., United States of America); Arlacel A; oeleic acid; tetanus helper peptides (such as but not limited to QYIKANSKFIGITEL (SEQ ID NO: 96) and/or AQYIKANSKFIGITEL (SEQ ID NO: 97); GM-CSF; cyclophosamide; bacillus Calmette-Guérin (BCG); Corynbacterium parvum; levamisole, azimezone; isoprinisone; dinitrochlorobenezene (DNCB); keyhole limpet hemocyanin (KLH); Freunds adjuvant (complete and incomplete); mineral gels; aluminum hydroxide (Alum); lysolecithin; pluronic polyols; polyanions; peptides; oil emulsions; nucleic acids (such as but not limited to double-stranded RNAs; dsRNA) dinitrophenol; diphtheria toxin (DT); toll-like receptor (TLR; such as but not limited to TLR3, TLR4, TLR7, TLR8, and/or TLR9) agonists (including but not limited to endotoxins such as lipopolysaccharide (LPS); monophosphoryl lipid A (MPL); and/or polyinosinic-polycytidylic acid (poly-ICLC/HILTONOL®; Oncovir, Inc., Washington, D.C., United States of America); IMO-2055; glucopyranosyl lipid A (GLA); QS-21 (a saponin extracted from the bark of the Quillaja saponaria tree, also known as the soap bark tree or Soapbark); resiquimod (a TLR7/8 agonist); CDX-1401 (a fusion protein consisting of a fully human monoclonal antibody with specificity for the dendritic cell receptor DEC-205 linked to the NY-ESO-1 tumor antigen); Juvaris' Cationic Lipid-DNA Complex; Vaxfectin; and combinations thereof.

In some embodiments, an adjuvant is a tetanus helper peptide (alternatively referred to herein as a “tetanus peptide”). As used herein, the phrases “tetanus helper peptide” and “tetanus peptide” refer to a peptide that comprises an amino acid sequence that is a subsequence of a wild type tetanus toxoid protein, or a derivative thereof. As used herein, the phrase “derivative thereof” refers to a peptide that comprises an amino acid sequence that is in some embodiments at least 75%, in some embodiments at least 80%, in some embodiments at least 85%, in some embodiments at least 90%, in some embodiments at least 91%, in some embodiments at least 92%, in some embodiments at least 93%, in some embodiments at least 94%, in some embodiments at least 95%, in some embodiments at least 96%, in some embodiments at least 97%, in some embodiments at least 98%, or in some embodiments at least 99% identical to, or in some embodiments 100% identical to, a reference amino acid sequence. In some embodiments of a derivative of a tetanus peptide, the reference amino acid sequence is SEQ ID NO: 96 or a subsequence thereof, and in some embodiments of a derivative of a tetanus peptide, the reference amino acid sequence is SEQ ID NO: 97 or a subsequence thereof.

Thus, in some embodiments the tetanus peptide can be about or at least 10, 11, 12, 13, 14, 15, 16, 17, 18, 19, 20, 21, 22, 23, 24, 25, or more natural or non-natural amino acids in length. In some embodiments, the tetanus peptide is a fragment of a wild type and/or naturally occurring tetanus toxoid protein (i.e., comprises an amino acid sequence that is a subsequence of the amino acid sequence of a wild type and/or naturally occurring tetanus toxoid protein). In some embodiments the tetanus toxoid peptide used herein is at least or about 70, 75, 80, 85, 90, 95, 98, 99% identical to a 10, 11, 12, 13, 14, 15, 16, 17, 18, 19, 20, 21, 22, 23, 24, or 25 amino acids segment of the wild type and/or naturally occurring tetanus toxoid protein. In some embodiments the tetanus toxoid peptide used herein is at least or about 70, 75, 80, 85, 90, 95, 98, 99% identical to SEQ ID NOs: 96 or 97. In some embodiments, the tetanus peptide is binds to MHC Class II molecules. In some embodiments, the tetanus peptide is modified so as to prevent formation of tetanus peptide secondary structures.

Polyinosinic-Polycytidylic acid (Poly IC) is a double-stranded RNA (dsRNA) that acts as a TLR3 agonist. To increase half-life, it has been stabilized with polylysine and carboxymethylcellulose as poly-ICLC. It has been used to induce interferon in cancer patients, with intravenous doses up to 300 μg/kg. Like poly-IC, poly-ICLC is a TLR3 agonist. TLR3 is expressed in the early endosome of myeloid DC; thus poly-ICLC preferentially activates myeloid dendritic cells, thus favoring a Th1 cytotoxic T cell response. Poly-ICLC activates natural killer (NK) cells, induces cytolytic potential, and induces IFNγ from myeloid DC.

In some embodiments, an adjuvant is provided at about or at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg per dose or per kg in each dose. In some embodiments, the adjuvant is provided in a dosage of at least or about 0.1, 0.2, 0.3, 0.40, 0.50, 0.60, 0.70, 0.80, 0.90, 0.100, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, 5.00, 5.10, 5.20, 5.30, 5.40, 5.50, 5.60, 5.70, 5.80, 5.90, 6.00, 6.10, 6.20, 6.30, 6.40, 6.50, 6.60, 6.70, 6.80, 6.90, 7.00, 7.10, 7.20, 7.30, 7.40, 7.50, 7.60, 7.70, 7.80, 7.90, 8.00, 8.10, 8.20, 8.30, 8.40, 8.50, 8.60, 8.70, 8.80, 8.90, 9.00, 9.10, 9.20, 9.30, 9.40, 9.50, 9.60, 9.70, 9.80, 9.90, or 10.00 grams per dose or per kg in each dose. In some embodiments, the adjuvant is given at about or at least 10, 15, 20, 25, 50, 75, 100, 125, 150, 175, 150, 175, 200, 225, 250, 275, 300, 325, 350, 375, 400, 425, 450, 500, 525, 550, 575, 600, 625, 675, 700, 725, 750, 775, 800, 900, 1000, 1100, 1200, 1300, 1400, 1500, 1600, 1700, 1800, 1900, or 2000 endotoxin units (“EU”) per dose. The target peptide compositions of the presently disclosed subject matter can in some embodiments be provided with an administration of cyclophosamide around the time (e.g., about or at least 1, 2, 3, or 4 weeks or days before and/or after) of the initial dose of a target peptide composition. Exemplary non-limiting doses of cyclophosamide are about or at least 100, 200, 300, 400, 500, 600, 700, 800, 900, or 1000 Mg/m²/day over about or at least 1, 2, 3, 4, 5, 6, 7, 8, 9, or 10 days.

The compositions can comprise the target peptides in the free form and/or in the form of a pharmaceutically acceptable salt. As used herein, “a pharmaceutically acceptable salt” refers to a derivative of a disclosed target peptide wherein the target peptide is modified by making acid or base salts of the agent. For example, acid salts are prepared from the free base (typically wherein the neutral form of the drug has a neutral —NH₂ group) involving reaction with a suitable acid. Suitable acids for preparing acid salts include both organic acids such as but not limited to acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids such as but not limited to hydrochloric acid, hydrobromic acid, sulfuric acid, nitric acid phosphoric acid, and the like. Conversely, basic salts of acid moieties that can be present on a target peptide are in some embodiments prepared using a pharmaceutically acceptable base such as but not limited to sodium hydroxide, potassium hydroxide, ammonium hydroxide, calcium hydroxide, trimmethylamine, and the like. By way of example and not limitation, the compositions can comprise target peptides as salts of acetic acid (acetates), ammonium, or hydrochloric acid (chlorides).

In some embodiments, a composition can include one or more sugars, sugar alcohols, amino acids such but not limited to glycine, arginine, glutamic acid, and/or others as framework formers. The sugars can be mono-, di-, or trisaccharides. These sugars can be used alone and/or in combination with sugar alcohols. Exemplary sugars include glucose, mannose, galactose, fructose, or sorbose as monosaccharides;

sucrose, lactose, maltose, and trehalose as disaccharides; and raffinose as a trisaccharide. A sugar alcohol can be, for example, mannitose. In some embodiments, the composition comprises sucrose, lactose, maltose, trehalose, mannitol, and/or sorbitol. In some embodiments, the composition comprises mannitol.

Furthermore, in some embodiments compositions can include physiological well-tolerated excipients (see Rowe et al., 2006) such as antioxidants like ascorbic acid or glutathione; preserving agents such as phenol, m-cresol, methyl- or propylparabene, chlorobutanol, thiomersal/thimerosal, and/or benzalkoniumchloride; stabilizers, framework formers such as sucrose, lactose, maltose, trehalose, mannitose, mannitol, and/or sorbitol; mannitol and/or lactose and solubilizers such as polyethylene glycols (PEG; e.g., PEG 3000, 3350, 4000, or 6000), cyclodextrins (e.g., hydroxypropyl-β-cyclodextrin, sulfobutylethyl-β-cyclodextrin, or y-cyclodextrin), dextranes, or poloxamers (e.g., poloxamer 407 or poloxamer 188); or TWEEN® 20 or TWEEN® 80. In some embodiments, one or more well-tolerated excipients can be included, optionally selected from the group consisting of antioxidants, framework formers, and stabilizers.

In some embodiments, the pH for intravenous and/or intramuscular administration is selected from pH 2 to pH 12. In some embodiments, the pH for subcutaneous administration is selected from pH 2.7 to pH 9.0 as the rate of in vivo dilution is reduced resulting in more potential for irradiation at the injection site (Strickley, 2004).

V.C. Dosages

It is understood that a suitable dosage of a target peptide composition vaccine immunogen cam depend upon the age, sex, health, and/or weight of the recipient, the kind of concurrent treatment, if any, the frequency of treatment, and the nature of the effect desired. However, it is understood that dosages can be tailored to the individual subject, as determined by the researcher or clinician. The total dose required for any given treatment will in some embodiments be determined with respect to a standard reference dose based on the experience of the researcher or clinician, such dose being administered either in a single treatment or in a series of doses, the success of which will depend on the production of a desired immunological result (such as but not limited to successful production of a T helper cell and/or CTL-mediated response to the target peptide immunogen composition, which response gives rise to the prevention and/or treatment desired).

Thus, in some embodiments the overall administration schedule is considered in determining the success of a course of treatment and not whether a single dose, given in isolation, would or would not produce the desired immunologically therapeutic result or effect. As such, a therapeutically effective amount (i.e., in some embodiments that amount that produces a desired T helper cell and/or CTL-mediated response) can depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and/or the sound judgment of the clinician or researcher. In some embodiments, the efficacy of administering additional doses and/or of increasing or decreasing the interval can be continually re-evaluated in view of the recipient's immunocompetence (including but not limited to the level of T helper cell and/or CTL activity with respect to tumor-associated or tumor-specific antigens).

The concentration of the T helper or CTL stimulatory target peptides of the presently disclosed subject matter in pharmaceutical formulations can be subject to wide variation, including anywhere from less than 0.01% by weight to as much as 50% or more. Factors such as volume and viscosity of the resulting composition can in some embodiments also be considered. The solvents or diluents used for such compositions can include water, phosphate buffered saline (PBS), and/or saline, or any other possible carriers or excipients.

The immunogens of the present presently disclosed subject matter can in some embodiments also be contained in artificially created structures such as liposomes, which structures in some embodiments can contain additional molecules such as but not limited to proteins or polysaccharides, inserted in the outer membranes of said structures and having the effect of targeting the liposomes to particular areas of the body and/or to particular cells within a given organ or tissue. Such targeting molecules can in some embodiments comprise an immunoglobulin. Antibodies can work particularly well for targeting of liposomes and/or other scaffolds to tumor cells.

Single i.d., i.m., s.c., i.p., and/or i.v. doses of in some embodiments about 1 to 50 in some embodiments about 1 to 100 in some embodiments about 1 to 500 μg, some embodiments about 1 to 1000 in some embodiments about 1 to 50 mg, in some embodiments about 1 to 100 mg, in some embodiments about 1 to 500 mg, or in some embodiments about 1 to 1000 mg of target peptide composition can be given and can depend from the respective compositions of target peptides with respect to total amount for all target peptides in the composition or alternatively for each individual target peptide in the composition. A single dose of a target peptide vaccine composition of the presently disclosed subject matter can in some embodiments have a target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 In some embodiments, a single dose of a target peptide composition of the presently disclosed subject matter can have a total target peptide amount (e.g., total amount for all target peptides in the composition or alternatively for each individual target peptide in the composition) of about or at least 1, 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 950 mg. In some embodiments, the target peptides of a composition of the presently disclosed subject matter are present in equal amounts of about 100 micrograms per dose in combination with an adjuvant peptide present in an amount of about 200 micrograms per dose.

In a single dose of the target peptide composition of the presently disclosed subject matter, the amount of each target peptide in the composition is in some embodiments equal or substantially equal. Alternatively, a ratio of the target peptides present in the least amount relative to the target peptide present in the greatest amount is about or at least 1:1.25, 1:1.5, 1:1.75, 1:2.0, 1:2.25, 1:2.5, 1:2.75, 1:3, 1:4, 1:5, 1:6, 1:7, 1:8, 1:9, 1:10, 1:20, 1:30; 1:40, 1:50, 1:100, 1:200, 1:500, 1:1000, 1:5000; 1:10,000; or 1:100,000. Alternatively, a ratio of the target peptides present in the least amount relative to the target peptide present in the greatest amount is about or at least 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15 to 20; 20 to 25; 1 to 40; 1 to 30; 1 to 20; 1 to 15; 10 to 40; 10 to 30; 10 to 20; 10 to 15; 20 to 40; 20 to 30; or 20 to 25; 1 to 100; 25 to 100; 50 to 100; 75 to 100; 25 to 75, 25 to 50, or 50 to 75; 25 to 40; 25 to 50; 30 to 50; 30 to 40; or 30 to 75.

Single dosages can be given to a patient about or at least 1, 2, 3, 4, or 5 times per day. Single dosages can be given to a patient about or at least 1, 2, 3, 4, 5, 6, 7, 8, 9, 10, 11, 12, 13, 14, 15, 16, 18, 19, 20, 21, 22, 23, 24, 36, 48, 60, or 72 hours subsequent to a previous dose.

Single dosages can be given to a patient about or at least 1, 2, 3, 4, 5, 6, or 7 times per week, or every other, third, fourth, or fifth day. Single doses can also be given every week, every other week, or only during 1, 2, or 3 weeks per month. A course of treatment can in some embodiments last about or at least 1, 2, 3, 4, 5, 6, 7, 8, 9, 10, 11, or 12 months.

In some embodiments, the single dosages of the compositions of the presently disclosed subject matter can be provided to a patient in at least two phases: e.g., during an initial phase and then during a subsequent phase. An initial phase can be about or at least 1, 2, 3, 4, 5, or 6 weeks in length. The subsequent phase can last at least or about 1, 2, 3, 4, 5, 6, 7, or 8 times as long as the initial phase. The initial phase can be separated from the subsequent phase by about or at least 1, 2, 3, 4, 5, 6, 7, 8, 9, 10, 11, or 12 weeks or months.

The target peptide composition dosage during the subsequent phase can be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times greater than during the initial phase.

The target peptide composition dosage during the subsequent phase can be at least 1, 2, 3, 4, 5, 6, 7, 8, 9, 10, 15, 20, 30, 40, 50, 60, 70, 80, 90, 100, 200, 300, 400, 500, 600, 700, 800, 900, or 1000 times less than during the initial phase.

In some embodiments, the initial phase is about three weeks and the second phase is about 9 weeks. The target peptide compositions can be administered to the patient on or about days 1, 8, 15, 36, 57, and 78.

V.D. Kits and Storage

In some embodiments, a kit is disclosed comprising (a) a container that contains at least one target peptide composition as described herein, in solution or in lyophilized form; (b) optionally, a second container containing a diluent or reconstituting solution for the lyophilized formulation; and (c) optionally, instructions for (i) use of the solution or (ii) reconstitution and/or use of the lyophilized formulation. The kit may further comprise one or more of (iii) a buffer, (iv) a diluent, (v) a filter, (vi) a needle, or (v) a syringe. In some embodiments, the container is selected from the group consisting of: a bottle, a vial, a syringe, a test tube, or a multi-use container. In some embodiments, the target peptide composition is lyophilized.

The kits can contain exactly, about, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, or more target peptide-containing compositions. Each composition in the kit can be administered at the same time or at different times.

In some embodiments, the kits can comprise a lyophilized formulation of the presently disclosed compositions and/or vaccines in a suitable container and instructions for its reconstitution and/or use. Suitable containers include, for example, bottles, vials (e.g., dual chamber vials), syringes (such as dual chamber syringes), and test tubes. The container can be formed from a variety of materials such as glass or plastic. In some embodiments, the kit and/or the container contain(s) instructions on or associated therewith that indicate(s) directions for reconstitution and/or use of a lyophilized formulation. For example, the label can indicate that the lyophilized formulation is to be reconstituted to target peptide concentrations as described herein. The label can further indicate that the formulation is useful or intended for subcutaneous administration. Lyophilized and liquid formulations are typically stored at −20° C. to −80° C.

The container holding the target peptide composition(s) can be a multi-use vial, which in some embodiments allows for repeat administrations (e.g., from 2-6 or more administrations) of the reconstituted formulation. The kit can further comprise a second container comprising a suitable diluent (e.g., sodium bicarbonate solution).

In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0, or 10 mg/mL/target peptide. In some embodiments, upon mixing of the diluent and the lyophilized formulation, the final peptide concentration in the reconstituted formulation is at least or about 0.15, 0.20, 0.25, 0.5, 0.75, 1.0, 1.25, 1.5, 1.75, 2.0, 2.25, 2.5, 2.75, 3.0, 3.25, 3.50, 3.75, 4.0, 4.25, 4.5, 4.75, 5.0, 6.0, 7.0, 8.0, 9.0, or 10 μg/mL/target peptide.

The kit can further include other materials desirable from a commercial and/or user standpoint, including other buffers, diluents, filters, needles, syringes, and package inserts with or without instructions for use.

The kits can have a single container that contains the formulation of the target peptide compositions with or without other components (e.g., other compounds or compositions of these other compounds) or can have a distinct container for each component.

Additionally, the kits can include a formulation of the presently disclosed target peptide compositions and/or vaccines packaged for use in combination with the co-administration of a second compound (such as adjuvants including but not limited to imiquimod), a chemotherapeutic agent, a natural product, a hormone or antagonist, an anti-angiogenesis agent or inhibitor, an apoptosis-inducing agent or a chelator) or a composition thereof. One or more of the components of the kit can be pre-complexed or one or more components can be in a separate distinct container prior to administration to a patient. One or more of the components of the kit can be provided in one or more liquid solutions. In some embodiments, the liquid solution is an aqueous solution. In a further embodiment, the liquid solution is a sterile aqueous solution. One or more of the components of the kit can also be provided as solids, which in some embodiments can be converted into liquids by addition of suitable solvents, which in some embodiments can be provided in another distinct container.

The container of a therapeutic kit can be a vial, a test tube, a flask, a bottle, a syringe, or any other structure suitable for enclosing a solid or liquid. Typically, when there is more than one component, the kit contains a second vial or other container that allows for separate dosing. The kit can also contain another container for a pharmaceutically acceptable liquid. In some embodiments, a therapeutic kit contains an apparatus (e.g., one or more needles, syringes, eye droppers, pipette, etc.), which enables administration of the agents of the disclosure that are components of the kit.

V.E. Markers for Efficacy

When administered to a patient, the vaccine compositions of the presently disclosed subject matter are in some embodiments envisioned to have certain physiological effects including but not limited to the induction of a T cell mediated immune response.

V.E.1. Immunohistochemistry, Immunofluorescence, Western Blots, Flow Cytometry

Validation and testing of antibodies for characterization of cellular and molecular features of lymphoid neogenesis has been performed. Commercially available antibodies for use in immunohistochemistry (IHC), immunofluorescence (IF), flow cytometry (FC), and/or western blotting (WB) can be used. In some embodiments, such techniques can be employed to assay patient samples including but not limited to formalin-fixed, paraffin-embedded tissue samples for the presence or absence of and/or for a level of expression of one or more of CD1a, S100, CD83, DC-LAMP, CD3, CD4, CD8, CD20, CD45, CD79a, PNAd, TNFα, LIGHT, CCL19, CCL21, CXCL12, TLR4, TLR7, FoxP3, PD-1, and Ki67 gene products. In some embodiments, flow cytometry is used to determine an expression level for one or more of CD3, CD4, CD8, CD13, CD14, CD16, CD19, CD45RA, CD45RO, CD56, CD62L, CD27, CD28, CCR7, FoxP3 (intracellular), and MHC-peptide tetramers for I MHC associated (O-GlcNAcylated)-peptides. In some embodiments, a positive control is employed, which in some embodiments can comprise a tissue sample comprising normal human peripheral blood lymphocytes (PBL), PBL activated with CD3/CD28 beads (activated PBL), human lymph node tissue from non-leukemia patients (LN), and/or inflamed human tissue from a surgical specimen of Crohn's disease (Crohn's), although any other positive control cell and/or tissue can be employed.

V.E.2 ELISpot Assay

In some embodiments, vaccination site infiltrating lymphocytes and lymphocytes from the sentinel immunized node (SIN) and vaccine site can be evaluated by ELISpot. ELISpot permits the direct counting of T cells reacting to antigen by production of IFNγ. Peripheral blood lymphocytes can be evaluated by ELISpot assay for the number of peptide-reactive T cells. Vaccine site infiltrating lymphocytes and SIN lymphocytes can be compared to those in peripheral blood. It is envisioned that positive results of the ELISpot assay correlates with increased patient progression free survival. Progression free survival is defined as the time from start of treatment until death from any cause or date of last follow up.

V.E.3 Tetramer Assay

Peripheral blood lymphocytes and lymphocytes from the SIN and vaccine site can be evaluated by flow cytometry after incubation with MHC-peptide tetramers for the number of peptide-reactive T cells.

V.E.4 Proliferation Assay/Cytokine Analysis

Peripheral blood mononuclear cells (PBMC), vaccine-site inflammatory cells, and/or lymphocytes from the SIN isolated from subjects can be evaluated for CD4⁺ T cell reactivity to, e.g., tetanus helper peptide mixture, using a ³H-thymidine uptake assay. Additionally, Th1 (IL-2, IFNγ, TNFα), Th2 (IL-4, IL-5, IL-10), Th17 (IL-17, and IL23), and T-reg (TGF-β) cytokines in media from 48 hours in that proliferation assay can be used to determine if the microenvironment supports generation of Th1, Th2, Th17, and/or T-reg responses. In some embodiments, one or both of the following peptides are used as negative controls: a tetanus peptide and a Pan HLA DR-binding Epitope (PADRE; e.g., AKFVAAWTLKAAA (SEQ ID NO: 98; see also U.S. Pat. No. 9,249,187).

V.E.5 Evaluation of Tumors

In some embodiments, tumor tissue collected prior to treatment or at the time of progression can be evaluated by routine histology and immunohistochemistry. Alternatively or in addition, in vitro evaluations of tumor tissue and tumor infiltrating lymphocytes can be performed.

V.E.6 Studies of Homing Receptor Expression

Patient samples can be studied for T cell homing receptors induced by vaccination with the compositions of the presently disclosed subject matter. These include, but are not limited to, integrins (including but not limited to αEƒ7, α1β1, α4β1), chemokine receptors (including but not limited to CXCR3), and selectin ligands (including but not limited to CLA and PSL) on lymphocytes, and their ligands in the vaccine sites and SIN. In some embodiments, these can be assayed by immunohistochemistry, flow cytometry, and/or any other appropriate technique(s).

V.E.7 Studies of Gene and Protein Expression

Differences in gene expression and/or differences in protein expression profiles can be determined by high-throughput screening assays (e.g., nucleic acid chips, protein arrays, etc.) of samples isolated from vaccine sites and/or SIN.

### VI. Antibodies and Antibody-Like Molecules

Antibodies and antibody-like molecules (including but not limited to T cell receptors) specific for target peptides and/or target peptide/MHC complexes are in some embodiments useful as therapeutics. In some embodiments, such molecules can be used as therapeutics that target cells, including but not limited to tumor cells, which display target peptides (e.g., the target peptides set forth in SEQ ID NOs:1-45, herein) on their surfaces.

The antibodies and antibody-like molecules disclosed herein can also be used for analyzing biological samples. In some embodiments, an analysis can comprise determining the pathological nature of tumor margins.

As used herein, the terms “antibody” and “antibody peptide” refer to an intact antibody, a binding fragment thereof (i.e., a fragment of an antibody that comprises a paratope), or a polypeptide that can specifically recognize an antigen or epitope and bind to the same in a fashion that mimics antibody binding. In some embodiments, antibodies and antibody peptides compete with intact antibodies for specific binding to an antigen or epitope.

The term “antibody” is used in the broadest sense, and covers monoclonal antibodies (including full length monoclonal antibodies), polyclonal antibodies, multispecific antibodies (e.g., bispecific and/or trispecific antibodies), and antibody fragments (including but not limited to Fab, F(ab′)₂ and Fv fragments) as well as antibody-like molecules provided that they exhibit the desired biological activity (e.g., antigen binding). In some embodiments, antibody fragments can be produced by recombinant DNA techniques and/or by enzymatic and/or chemical cleavage of intact antibodies. Antibody fragments thus include but are not limited to Fab fragments, Fab′ fragments, F(ab′)₂ fragments, Fv, and single-chain antibodies including but not limited to single-chain fragment variable (scFv) antibodies. An antibody is said to be “monospecific” if each of its paratopes is identical and/or binds to the same epitope. Similarly, “bispecific” or “bifunctional” antibodies comprise paratopes that bind to different antigens and/or epitopes. In some embodiments, an antibody substantially inhibits adhesion of a receptor to a counterreceptor when an excess of antibody reduces the quantity of receptor bound to counterreceptor by at least about 20%, 40%, 60%, 80%, 85%, 90%, 95%, or more as measured by, for example, an in vitro competitive binding assay.

As used herein, the phrase “antibody-like molecule(s)” refers to to an intact protein, a binding fragment thereof, or a polypeptide that can specifically recognize an antigen or epitope and bind to the same in a fashion that mimics antibody or T-cell receptor binding to the antigen or epitope.

In some embodiments, the antibody-like molecule is a T-cell receptor. As used herein, the terms “T cell receptor” and “TCR” are used interchangeably and refer to full length heterodimeric αβ or γδ TCRs, antigen-binding fragments of TCRs, or molecules comprising TCR CDRs or variable regions. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, monospecific TCRs, multi-specific TCRs (including bispecific TCRs), TCR fusions, human TCRs, humanized TCRs, chimeric TCRs, recombinantly produced TCRs, and synthetic TCRs. The term encompasses wild-type TCRs and genetically engineered TCRs (e.g., a chimeric TCR comprising a chimeric TCR chain which includes a first portion from a TCR of a first species and a second portion from a TCR of a second species).

As used herein, the term “TCR variable region” is understood to encompass amino acids of a given TCR which are not included within the non-variable region as encoded by the TRAC gene for TCR a chains and either the TRBC1 or TRBC2 genes for TCR β chains. In some embodiments, a TCR variable region encompasses all amino acids of a given TCR which are encoded by a TRAV gene or a TRAJ gene for a TCR α chain or a TRBV gene, a TRBD gene, or a TRBJ gene for a TCR β chain (see e.g., LeFranc & LeFranc, 2001, which is incorporated by reference herein in its entirety).

As used herein, the term “constant region” with respect to a TCR refers to the extracellular portion of a TCR that is encoded by the TRAC gene for TCR a chains and either the TRBC1 or TRBC2 genes for TCR θ chains. The term constant region does not include a TCR variable region encoded by a TRAV gene or a TRAJ gene for a TCR a chain or a TRBV gene, a TRBD gene, or a TRBJ gene for a TCR β chain (see e.g., LeFranc & LeFranc, 2001, which is incorporated by reference herein in its entirety).

In some embodiments, the T cell receptor is a soluble T cell receptor (TCR). See e.g., U.S. Patent Application Publication No. 2002/0119149, which is incorporated by reference. Such soluble TCRs can in some embodiments be conjugated to immunostimulatory peptides and/or proteins, and/or moieties such as but not limited to CD3 agonists (e.g., anti-CD3 antibodies). The CD3 antigen is present on mature human T cells, thymocytes, and a subset of natural killer cells. It is associated with the TCR and is involved in signal transduction of the TCR. Antibodies specific for the human CD3 antigen are well known. One such antibody is the murine monoclonal antibody OKT3 which was the first monoclonal antibody approved by the FDA. OKT3 is reported to be a potent T cell mitogen (Van Wauve, 1980; U.S. Pat. No. 4,361,539) and a potent T cell killer (Wong, 1990). Other antibodies specific for the CD3 antigen have also been reported (see PCT International Patent Application Publication No. WO 2004/106380; U.S. Patent Application Publication No. 2004/0202657; U.S. Pat. No. 6,750,325; U.S. Pat. No. 6,706,265; Great Britain Patent Publication GB 2249310A; Clark et al., 1989; U.S. Pat. No. 5,968,509; U.S. Patent Application Publication No. 2009/0117102). Immune mobilizing mTCR Against Cancer (ImmTAC; Immunocore Limited, Milton Park, Abington, Oxon, United Kingdom) are bifunctional proteins that combine affinity monoclonal T cell receptor (mTCR) targeting with a therapeutic mechanism of action (i.e., an anti-CD3 scFv).

The term “MHC” as used herein refer to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”. In murine species, the MHC is referred to as the “H-2” complex.

The terms “MHC light chain” and “MHC heavy chain” as used herein refer to particular portions of a MHC molecule. Structurally, class I molecules are heterodimers comprised of two noncovalently bound polypeptide chains, a larger “heavy” chain (a) and a smaller “light” chain (β2-microglobulin or β2m). The polymorphic, polygenic heavy chain (45 kDa), encoded within the MHC on chromosome human 6 is subdivided into three extracellular domains (designated 1, 2, and 3), one intracellular domain, and one transmembrane domain. The two outermost extracellular domains, 1 and 2, together form the groove that binds to antigenic peptides and/or other epitopes. Thus, interaction with the TCR occurs at this region of the protein. Domain 3 of the molecule contains the recognition site for the CD8 protein on the CTL. This interaction serves to stabilize the contact between the T cell and an antigen-presenting cell (APC). The invariant light chain (12 kDa), encoded on human chromosome 15, consists of a single, extracellular polypeptide. The terms “MHC light chain”, “β2-microglobulin”, and “β2m” are used interchangeably herein.

The term “epitope” includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three dimensional structural characteristics as well as specific charge characteristics. An antibody or antibody-like molecule is said to “specifically” bind an antigen when the dissociation constant (K_{d}) is in some embodiments less than about 1 in some embodiments less that about 100 nM, and in some embodiments less than about 10 nM. Interactions between antibodies and antibody-like molecules and an epitope can also be characterized by an affinity constant (Kₐ). In some embodiments, a Kₐ of less than about 10⁷/M is considered “high affinity”.

In some embodiments, antibodies or antibody-like molecules bind to the target peptides disclosed herein but do not substantially and/or specifically crossreact with the same peptide in a modified form. See e.g., U.S. Patent Application Publication No. 2009/0226474, which is incorporated by reference. In other embodiments, antibodies or antibody-like molecules specifically bind to O-GlcNAcylated and/or methylated target peptides and/or target peptide-MHC complex and do not substantially cross react with the corresponding non-O-GlcNAcylated and/or non-methylated native peptides. In some embodiments, provided are antibodies or antibody-like molecules that specifically bind to an O-GlcNAcylated and/or a methylated target peptide set forth in SEQ ID NOs:1-45 and Tables 3-7 herein, and/or specifically bind to a peptide-MHC complex comprising the target peptide (e.g., an O-GlcNAcylated and/or a methylated target peptide set forth in SEQ ID NOs:1-45 and Tables 3-7 bound to a cognate MHC, such as HLA A*0201, HLA B*0702, and HLA B*35, as indicated in Tables 3-7 herein), and do not substantially cross react with the corresponding non-O-GlcNAcylated and/or non-methylated native peptide. In some embodiments, provided are T cell receptors that specifically bind to an O-GlcNAcylated and/or a methylated target peptide set forth in SEQ ID NOs:1-45 and Tables 3-7 herein, and/or specifically bind to a peptide-MHC complex comprising the target peptide (e.g., an O-GlcNAcylated and/or a methylated target peptide set forth in SEQ ID NOs:1-45 and Tables 3-7 bound to a cognate MHC, such as HLA A*0201, HLA B*0702, and HLA B*35, as indicated in Tables 3-7 herein), and do not substantially cross react with the corresponding non-O-GlcNAcylated and/or non-methylated native peptide. In some embodiments, provided are cells (e.g., T-cells or Natural Killer Cells) expressing on their surface antibodies or antibody-like molecules (e.g., T cell receptors) that bind specifically to an O-GlcNAcylated and/or a methylated target peptide set forth in SEQ ID NOs:1-45 and Tables 3-7 herein, and/or specifically bind to a peptide-MHC complex comprising the target peptide (e.g., an O-GlcNAcylated and/or a methylated target peptide set forth in SEQ ID NOs:1-45 and Tables 3-7 bound to a cognate MHC, such as HLA A*0201, HLA B*0702, and HLA B*35, as indicated in Tables 3-7 herein), and do not substantially cross react with the corresponding non-O-GlcNAcylated and/or non-methylated native peptide.

The presently disclosed subject matter includes in some embodiments antibodies that recognize target peptides associated with a tumorigenic or disease state, wherein the peptides are displayed in the context of HLA molecules. These antibodies can mimic the specificity of a T cell receptor (TCR) but can have higher binding affinities such that the molecules can be employed as therapeutic, diagnostic, and/or research reagents. Methods of producing a T cell receptor mimic of the presently disclosed subject matter in some embodiments comprise identifying a target peptide of interest, generating an isolating CD8⁺ T cells comprising T cell receptors (TCRs) that are specific for the target peptide, and cloning the genomic sequences present in the isolated CD8⁺ T cells that encode the TCRs that are specific for the target peptide.

In some embodiments, an immunogen comprising at least one target peptide/MHC complex is formed. An effective amount of the immunogen is in some embodiments administered to a host to elicit an immune response in the host, and serum collected from the host can be assayed to determine if antibodies that recognize a three-dimensional presentation of the target peptide in the binding groove of the MHC molecule have been produced. The desired antibodies can in some embodiments differentiate the target peptide/MHC complex from the MHC molecule alone, the target peptide alone, and/or a complex of MHC and an irrelevant peptide (in some embodiments, a peptide having the same amino acid composition as a target peptide but wherein the amino acids are in a different order that in the target peptide). Finally, in some embodiments the desired antibodies can be isolated.

Native antibodies and immunoglobulins are generally heterotetrameric glycoproteins of about 150,000 daltons (Da) composed of two identical light (L) chains and two identical heavy (H) chains. Each light chain is linked to a heavy chain by a covalent disulfide bond. Disulfide bonds also link the heavy chains of intact antibodies, although the number of disulfide bonds between the heavy chains of different immunoglobulin isotypes can vary. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., 1985; Novotny & Haber, 1985).

An “isolated” antibody is one which has been identified and/or separated and/or recovered from a component of the environment in which it was produced or otherwise present. Contaminant components of its production environment are materials that in some embodiments interfere with diagnostic and/or therapeutic uses for the antibody, and in some embodiments can include enzymes, hormones, and other proteinaceous or nonproteinaceous solutes. In some embodiments, an antibody can be purified as measurable by one or more of the following methods: 1) to greater than 50%, 75%, 80%, 85%, 90%, 95%, 96%, 97%, 98%, or 99% by weight of antibody as determined by the Lowry method; 2) to a degree sufficient to obtain at least 10 or 15 residues of N-terminal or internal amino acid sequence by use of a spinning cup sequenator; or 3) to homogeneity by SDS-PAGE under reducing or non-reducing conditions using Coomassie blue or, in some embodiments, silver stain. Isolated antibodies include an antibody in situ within recombinant cells since at least one component of the antibody's natural environment will not be present. Ordinarily, however, isolated antibodies will be prepared by a method that comprises at least one purification step.

The terms “antibody mutant” and “antibody variant” refer to antibodies that relative to a reference antibody comprise one or more amino acid sequence differences, wherein one or more of the amino acid residues have been modified such as but not limited to substitution and/or deletion. Such mutants and/or variants comprise in some embodiments less than 100%, 99%, 98%, 97%, 96%, 95%, 90%, 85%, 80%, or 75% sequence identity and/or similarity to the amino acid sequence of either the heavy or light chain variable domain amino acid sequence of the reference antibody.

The term “variable” in the context of variable domain of antibodies, refers to the fact that certain portions of the variable domains differ extensively in sequence among antibodies and are used in the binding and specificity of each particular antibody for its particular antigen. However, sequence variability is generally not evenly distributed throughout the variable domains of antibodies. Typically, sequence variability is concentrated in three segments called complementarity determining regions (CDRs; also known as hypervariable regions) both in the light chain and heavy chain variable domains.

There are at least two techniques for determining CDRs: (1) an approach based on cross-species sequence variability (i.e., Kabat et al., 1991); and (2) an approach based on crystallographic studies of antigen-antibody complexes (Chothia et al., 1989). The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al., 1991) The constant domains are generally not involved directly in binding between antibody and antigen, but exhibit various effector functions such as but not limited to participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂, and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, the phrase “functional fragment” with respect to antibodies refers in some embodiments to a fragment that contains at least one antigen-binding domain (referred to as a “paratope”), and thus includes, but is not limited to Fv, F(ab) and F(ab′)₂ fragments.

An “Fv” fragment is the minimum antibody fragment which contains a complete antigen recognition and binding site. This region consists of a heterodimer of one heavy and one light chain variable domain in a tight, non-covalent or covalent association (V_{H}-V_{L} dimer). It is in this configuration that the three CDRs of each variable domain interact to define an antigen binding site (paratope) on the surface of the V_{H}-V_{L} dimer. Collectively, the six CDRs confer antigen binding specificity to the antibody. However, in some embodiments even a single variable domain (or half of an Fv comprising only three CDRs specific for an antigen) has the ability to recognize and bind antigen, although at a lower affinity than the entire binding site.

The Fab or F(ab) fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of the corresponding constant domain.

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses or isotypes (e.g., IgG₁, IgG₂, IgG₃, IgG₄, IgA₁, IgA₂, etc.). The heavy chains constant domains that correspond to the different classes of immunoglobulins are called alpha (a), delta (A), epsilon (c), gamma (y), and mu (μ), respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, monoclonal antibodies can be advantageous in that they are typically synthesized from hybridomas and thus can be isolated in a form that is uncontaminated by other immunoglobulins. Methods for generating hybridomas are known in the art. The modifier “monoclonal” indicates the character of the antibody as being obtained from a substantially homogeneous population of antibodies, and is not to be construed as requiring production of the antibody by any particular method. By way of example and not limitation, monoclonal antibodies to be used in accordance with the presently disclosed subject matter can be made by the hybridoma method first described by Kohler & Milstein, 1975, or can be made by recombinant methods (see e.g., U.S. Pat. No. 4,816,567; Harlow & Lane, 1988). In some embodiments, the monoclonal antibodies for use with the presently disclosed subject matter can be isolated from phage antibody libraries using the techniques described in Clackson et al., 1991 and/or Marks et al., 1991.

Utilization of the monoclonal antibodies of the presently disclosed subject matter can in some embodiments comprise administering one or more monoclonal antibodies to a subject, such as but not limited to a human subject. However, when the monoclonal antibodies are produced in a non-human animal, such as a rodent, administration of such antibodies to a human patient can elicit an immune response, wherein the immune response is directed towards the administered antibodies themselves. Such reactions can limit the duration and effectiveness of such a therapy. In order to overcome such a problem, the monoclonal antibodies of the presently disclosed subject matter can in some embodiments be “humanized”, that is, the antibodies are engineered such that antigenic portions thereof are removed and like portions of a human antibody are substituted therefor, while the antibodies' affinity for specific peptide/MHC complexes is retained. This engineering can involve a few amino acids, or can include the entire framework regions of the antibody, leaving only the complementarity determining regions of the parent antibody intact. Several methods of humanizing antibodies are known in the art and are disclosed in U.S. Pat. Nos. 6,180,370; 6,054,927; 5,869,619; 5,861,155; 5,712,120; and 4,816,567, the entire disclosure of each of which is hereby expressly incorporated herein by reference in its entirety.

Humanized forms of antibodies are thus chimeric immunoglobulins, immunoglobulin chains, or fragments thereof (such as Fv, Fab, Fab′, F(ab′)₂ or other antigen-binding subsequences of antibodies) that are principally comprised of the sequence of a human immunoglobulin, but that contain at least some subsequences derived from a non-human immunoglobulin. Humanization can be performed following the method of Winter and co-workers by substituting rodent CDRs or CDR sequences for the corresponding sequences of a human antibody (Jones et al., 1986; Riechmann et al., 1988; Verhoeyen et al., 1988; see also U.S. Pat. No. 5,225,539). In some embodiments, F, framework residues of a human immunoglobulin are replaced with corresponding non-human residues from an antibody of interest. Humanized antibodies can also comprise residues which are found neither in the recipient antibody nor in the imported CDR or framework sequences. In general, the humanized antibody comprises substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally can also comprise at least a portion of an immunoglobulin constant region (Fc), typically that of a human immunoglobulin (Jones et al., 1986; Riechmann et al., 1988; and Carter et al., 1992).

Exemplary publications relating to the generation and/or use of humanized antibodies include Sandborn et al., 2001; Mihara et al., 2001; Yenari et al., 2001; Morales et al., 2000; Richards et al., 1999; Yenari et al., 1998; and Shinkura et al., 1998; each of which is expressly incorporated by reference herein in its entirety. For example, a treatment protocol that can be utilized in such a method includes a single dose, generally administered intravenously, of 10-20 mg of humanized mAb per kg (see e.g., Sandborn et al., 2001). In some cases, alternative dosing patterns can be appropriate, such as the use of three infusions, administered once every two weeks, of 800-1600 mg or even higher amounts of humanized mAb (see e.g., Richards et al., 1999). However, it is to be understood that the presently disclosed subject matter is not limited to the treatment protocols described herein, and further that other treatment protocols that are known to one of ordinary skill in the art can be employed in the methods of the presently disclosed subject matter.

In some embodiments, the presently disclosed subject matter further relates to fully human monoclonal antibodies against specific target peptide/MHC complexes. Fully human antibodies essentially relate to antibody molecules in which the entire sequence of both the light chain and the heavy chain, including the CDRs, arise from human genes. Such antibodies are referred to herein as “human antibodies” or “fully human antibodies”.

Human monoclonal antibodies can be prepared by the trioma technique (see U.S. Pat. No. 4,714,681; PCT International Patent Application Publication No. WO 1999/047929); the human B-cell hybridoma technique (see Kozbor et al., 1983), and/or the EBV hybridoma technique (see Cole et al., 1985). In some embodiments, human monoclonal antibodies can be utilized in the practice of the presently disclosed subject matter and can be produced by using human hybridomas (see Cote et al., 1983) or by transforming human B-cells with Epstein Barr Virus in vitro (see Cole et al., 1985). In addition, human antibodies can also be produced using additional techniques, such as but not limited to phage display libraries (Hoogenboom et al., 1991; Marks et al., 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., 1992; Lonberg et al., 1994; Fishwild et al., 1996; Neuberger, 1996; and Lonberg & Huszar, 1995.

Human antibodies can additionally be produced using transgenic non-human animals that have been modified to produce fully human antibodies in addition to or rather than the non-human animal's endogenous antibodies in response to challenge by an antigen. See PCT International Patent Application Publication No. WO 1994/02602. In some embodiments, endogenous genes encoding the heavy and light immunoglobulin chains present in the non-human animal have been deleted or otherwise inactivated, and nucleic acids encoding human heavy and light chain immunoglobulins have been inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications.

One embodiment of such a non-human animal is a mouse termed the XENOMOUSE™, which is described in PCT International Patent Application Publication Nos. WO 1996/33735 and WO 1996/34096. The XENOMOUSE′ produces B cells that secrete fully human immunoglobulins. The antibodies can be obtained directly from the animal after immunization with an immunogen of interest, as, for example, a preparation of polyclonal antibodies, or alternatively from immortalized B cells derived from an immunized animal, such as hybridomas producing monoclonal antibodies. Additionally, the genes encoding the immunoglobulins with human variable regions can be recovered and expressed to obtain the antibodies directly and/or can be further modified to obtain analogs of antibodies such as, for example, single chain Fv molecules.

An example of a method for producing a non-human animal such as but not limited to a mouse that lacks expression of an endogenous immunoglobulin heavy chain is disclosed in U.S. Pat. No. 5,939,598, incorporated herein by reference. Such a non-human animal can be obtained by a method that comprises deleting the J segment genes from at least one endogenous heavy chain locus in an embryonic stem cell, thereby preventing rearrangement of the locus and formation of an RNA encoding a rearranged immunoglobulin heavy chain locus. In some embodiments, the deletion can be effected by a targeting vector that contains a selectable marker, Thereafter, a transgenic animal (e.g., a mouse) having somatic and germ cells containing the gene encoding the selectable marker can be produced from the embryonic stem cell. The transgenic animal would be expected to be unable to rearrange its endogenous immunoglobulin heavy chain locus, and thus would be expected to be unable to produce endogenous immunoglobulins.

A method for producing an antibody of interest, such as a human antibody, is also disclosed in U.S. Pat. No. 5,916,771, incorporated herein by reference. It includes introducing a first expression vector that contains a nucleotide sequence encoding a heavy chain into one mammalian host cell in culture, introducing a second expression vector containing a nucleotide sequence encoding a light chain into another mammalian host cell, and fusing the two cells to form a hybrid cell. The hybrid cell can express thus an antibody made up of a heavy chain and a light chain encoded by the first and second expression vectors.

Target peptides disclosed herein are in some embodiments expressed on a variety of cancer cell types. Thus, in some embodiments antibodies and antibody-like molecules can be used in treating, diagnosing, vaccinating, preventing, retarding, and attenuating a cancer such as but not limited to melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

Antibodies and/or antibody-like molecules generated with specificity for a target peptide as disclosed herein can be used to detect the corresponding target peptides in a biological sample. The biological sample is in some embodiments isolated from an individual who is suspected of having cancer, and thus detection could serve to diagnose the cancer. Alternatively, the biological sample could be isolated from an individual known to have cancer, and detection of a target peptide therein can serve as an indicator of disease prognosis, cancer characterization, treatment efficacy, disease progression, or any combination thereof. Immunoassays that can be employed for these purposes are known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine.

Antigens recognized by T cells, whether helper T lymphocytes or CTL, are not recognized as intact proteins, but rather as small peptides that associate with class I or class II MHC proteins on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules.

Antigens that give rise to proteins that are recognized in association with class I MHC molecules are generally proteins that are produced within the cells, and these antigens are processed and associate with class I MHC molecules. It is now understood that the peptides that associate with given class I or class II MHC molecules are characterized as having a common binding motif, and the binding motifs for a large number of different class I and II MHC molecules have been determined. Synthetic peptides can also be synthesized that correspond to the amino acid sequence of a given antigen and that contain a binding motif for a given class I or II MHC molecule. These peptides can then be added to appropriate APCs, and the APCs can be used to stimulate a T helper cell or CTL response either in vitro or in vivo. The binding motifs, methods for synthesizing the peptides, and methods for stimulating a T helper cell or CTL response are all known and readily available to one of ordinary skill in the art.

Kits can be prepared to assist in diagnosis, monitoring, and/or prognosis of diseases. In some embodiments, the kits facilitate the detection and/or measurement of cancer-specific O-GlcNAcylated peptides and/or O-GlcNAcylated proteins. Such kits can contain, in a single or divided container, a molecule comprising an antigen-binding region. In some embodiments, such molecules are antibodies or antibody-like molecules. Additional components that can be included in the kit include one or more of solid supports, detection reagents, secondary antibodies, instructions for use, vessels for running assays, gels, control samples, and the like. In some embodiments, an antibody or antibody-like molecules can optionally be directly or indirectly labeled.

Alternatively, the antibody or antibody-like molecules specific for O-GlcNAcylated peptides and/or O-GlcNAcylated peptide/MHC complexes can be conjugated to therapeutic agents. Exemplary therapeutic agents include, but are not limited to the following:

Alkylating Agents:

Alkylating agents are drugs that directly interact with genomic DNA to prevent cells from proliferating. This category of chemotherapeutic drugs represents agents that affect all phases of the cell cycle (i.e., they are not cell cycle phase-specific). Alkylating agents include, but are not limited to nitrogen mustards, ethylenimenes, methylmelamines, alkyl sulfonates, nitrosoureas, and triazines. Particularly exemplary alkylating agents include but are not limited to busulfan, chlorambucil, cisplatin, cyclophosphamide (cytoxan), dacarbazine, ifosfamide, mechlorethamine (mustargen), and melphalan.

Antimetabolites:

Antimetabolites disrupt DNA and RNA synthesis. Unlike alkylating agents, they specifically influence the cell cycle during S phase. Antimetabolites can be differentiated into various categories, such as folic acid analogs, pyrimidine analogs, purine analogs, and related inhibitory compounds. Antimetabolites include but are not limited to 5-fluorouracil (5-FU), cytarabine (Ara-C), fludarabine, gemcitabine, and methotrexate.

Natural Products:

Natural products generally refer to compounds originally isolated from a natural source and identified as having a desirable pharmacological activity. Such compounds, including analogs and derivatives thereof, can be isolated from a natural source, chemically synthesized, and/or recombinantly produced by any technique known to those of skill in the art. Natural products include such categories as mitotic inhibitors, antitumor antibiotics, enzymes, and biological response modifiers.

Mitotic inhibitors include plant alkaloids and other natural agents that can in some embodiments inhibit protein synthesis required for cell division and in some embodiments inhibit mitosis. They typically operate during a specific phase of the cell cycle. Mitotic inhibitors include, for example, docetaxel, etoposide (VP16), teniposide, paclitaxel, taxol, vinblastine, vincristine, and vinorelbine, among others.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. Exemplary vinca alkaloids include vinblastine (VLB) and vincristine.

Antibiotics:

Certain antibiotics have both antimicrobial and/or cytotoxic activity. These drugs also interfere with DNA by chemically inhibiting enzymes and mitosis or altering cellular membranes. These agents are typically not cell cycle phase-specific. Examples of cytotoxic antibiotics include but are not limited to bleomycin, dactinomycin, daunorubicin, doxorubicin (Adriamycin), plicamycin (mithramycin), and idarubicin.

Miscellaneous Agents:

Miscellaneous cytotoxic agents that do not fall into the previous categories include but are not limited to platinum coordination complexes, anthracenediones, substituted ureas, methyl hydrazine derivatives, amsacrine, L-asparaginase, and tretinoin. Platinum coordination complexes include such compounds as carboplatin and cisplatin (cis-DDP). An exemplary anthracenedione is mitoxantrone. An exemplary substituted urea is hydroxyurea. An exemplary methyl hydrazine derivative is procarbazine (N-methylhydrazine, MIH). These examples are non-limiting and it is contemplated that any known cytotoxic, cytostatic, and/or cytocidal agent can be attached to a targeting peptide of the presently disclosed subject matter and administered to a targeted organ, tissue, and/or cell type.

Chemotherapeutic (cytotoxic) agents including, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine, methotrexate, vincristine, and any analogs and/or derivatives or variants of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog, derivative, or variant thereof.

### EXAMPLES

The following Examples provide further illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

Leukemia Samples and Cell Lines.

Leukemia samples were those described in Cobbold et al., 2013. All cell lines were grown at 37° C. with 5% CO₂ in medium containing RPMI 1640 supplemented with 10% fetal bovine serum and 2 mM L-glutamine (all from Sigma-Aldrich Co. LLC., St. Louis, Mo., United States of America).

Isolation of HLA-Associated Peptides.

Class I MHC molecules were immunoaffinity-purified from cell lines or tumors and their associated peptides were extracted as described in Cobbold et al., 2013. 10⁸-10⁹ cells were lysed in 10 mL of CHAPS buffer (Sigma-Aldrich Co. LLC., St. Louis, Mo., United States of America) and the lysate was centrifuged at 100,000 g for 1 hour at 4° C. Supernatants were passed over protein A-SEPHAROSE® preloaded with either HLA-A2 specific antibody (BB7.2), HLA-B7 specific antibody (ME1), or HLA-A/B/C nonspecific antibody (W6/32). Peptides were eluted from the purified MHC class I molecules with 10% acetic acid and separated by ultrafiltration (ULTRAFREE®-MC, Millipore, Billerica, Mass., United States of America). See also U.S. Patent Application Publication No. 2015/0224182 and PCT International Patent Application Serial No. PCT/US2013/057856 (published as PCT International Patent Application Publication No. WO 2014/036562).

Enrichment of HLA-Associated Peptides.

POROS® 20 AL beads (Applied Biosystems, Carlsbad, Calif., United States of America) were derivatized with amino-phenyl boronic acid (APBA; Thermo Fisher Scientific, Waltham, Mass., United States of America). Briefly, POROS® 20 beads (7 mg) were dispersed into 200 μL of PBS (pH 6-7) containing 40 μmol of APBA. Following the addition of NaCNBH₃ (1.3 μmol in 1 μL of PBS), the reaction was allowed to proceed with agitation for 2 hours at room temperature (RT) and then quenched by washing the beads with water on a spin column (pore size <20 μm). Water was removed under vacuum and the dried beads were stored at 4° C.

Class I MHC peptides from 2-5×10⁸ cells in 0.1% acetic acid were desalted by loading the solution onto a fused-silica column (360 μm o.d.×150 μm i.d.) packed in-house with 5 cm of irregular C18 (5-20 μm diameter) particles at a flow rate of 0.5 μL/min. After washing the column with 25 μL of 0.1% acetic acid, peptides were eluted into Eppendorf tubes with a 40 minute gradient (0-80%) of solvent B (A: 0.1M acetic acid; B: 70% acetonitrile, 0.1M acetic acid). Fractions were screened by mass spectrometry (MS) and those that contained peptides, but not CHAPS detergent, were combined, taken to dryness, and stored at −35° C.

APBA-beads were washed 3× with 100 μL portions of anhydrous dimethylformamide (DMF) and then allowed to react with desalted peptides in 20 of anhydrous DMF for 1 hour with agitation. Solvent was removed by centrifugation and the beads were washed 2× with 100 μL of anhydrous acetonitrile. Bound peptides were released by agitating the beads in 20 μL of 0.1M acetic acid for 30 minutes. Supernatant was collected, taken to dryness, and reconstituted in 10 μL of 0.1M acetic acid for loading onto an in-house packed C18 column for MS analysis.

RP-HPLC-Mass Spectrometry.

In-house, packed C18 columns were prepared as previously described (Udeshi et al., 2008; see also Cobbold et al., 2013). Peptides were eluted by a 2 hour 0-60% B gradient (A: 0.1M acetic acid; B: 70% ACN, 0.1M acetic acid). Without enrichment, samples were loaded directly onto the C18 column. The RP-HPLC elution was electrospray-ionized into an Orbitrap Velos, or Orbitrap Fusion Tribrid mass spectrometer (Thermo Scientific, San Jose, Calif.), the former equipped with an in-house front-end ETD ion source. On the Orbitrap Velos, the instrument method was a top-10 CAD with ETD only when loss of dehydro-GlcNAc neutral loss of [203]+2 or [203]+3 was detected. On the Fusion, instrument method was a top speed HCD triggered ETD when three of six O-GlcNAc fingerprint ions (m/z 204, 186, 168, 144, 138, and 126) were detected at >5% relative abundance. Peptide sequences were determined by manual interpretation of CAD and ETD mass spectra.

Synthetic Peptides.

O-GlcNAc- and O-GalNAc-peptides were synthesized using Fmoc chemistry and purified by HPLC to >90% purity by Pierce Biotechnology, Rockford, Ill., United States of America. Sequences and purity for all synthetic peptides were confirmed in-house by on-line HPLC tandem mass spectrometry (MS/MS) and manual interpretation of the resulting spectra.

Selective Transfer of N-Azidoacetylgalactosamine (GalNAz) to O-GlcNAcylated Peptides.

A solution of the modified β1-4-galactosyltransferase, GalT1 (Invitrogen, Carlsbad, Calif., United States of America), was dried to 5 μL in a vacuum concentrator at 40° C. Excess synthetic glycopeptides or tumor peptides (3×10⁸ cell equivalents of ALL) were taken to dryness, redissolved in a mixture containing 1 μL MnCl₂, 5 μL uridinediphosphate N-azidoacetylgalactosamine, UDP-GalNAz (Invitrogen, Carlsbad, Calif., United States of America), and 5 μL enzyme and allowed to react for 5 hours at room temperature. After the reaction was quenched by addition of 0.2 μL glacial acetic acid, the solution volume was increased to 15 μL with 0.1% acetic acid, and then loaded directly onto an HPLC column for analysis by LC-MS/MS.

Intracellular Cytokine Staining (ICS).

Peripheral blood mononuclear cells (PBMCs) were isolated from healthy donors (HDs) and resuspended (1×10⁶ cell equivalents/mL) in AIM-V® medium (Invitrogen, Carlsbad, Calif., United States of America). Synthetic peptide antigens were added to the wells (10 μg/mL) and cells were expanded for 6 days. The positive control was stimulated with phytohaemagglutinin (PHA; 1 μg/ml). On day 6, cells were washed and re-stimulated with peptide antigen overnight or, for the positive control, with PMA/Ionomycin (4 ng/ml and 500 ng/ml respectively), in the presence of anti-CD107a (Biolegend, Cambridge, United Kingdom). Cells were harvested, washed with PBS, and stained with fixable viability dye (APC-Cy7 conjugate; eBioscience, Hatfield, United Kingdom) and surface antibodies: anti-CD3 (allophycocyanin (APC) conjugate) and anti-CD8 (Peridinin Chlorophyll Protein Complex (PerCP) conjugate; Biolegend, Cambridge, United Kingdom). Cells were fixed using 2% formaldehyde, permeablized using 0.5% saponin, and stained with anti-IFNγ (Perkin Elmer, Conventry, United Kingdom), anti-IL2 (Pacific blue conjugate; BioLegend, Cambridge, United Kingdom), and anti-TNFα (PE-Cy5.5 conjugate; BioLegend, Cambridge, United Kingdom) for 30 minutes at RT. Cells were washed, lightly fixed, and analyzed on the LSRFORTESSA™ X-20 flow cytometer (BD Biosciences, Oxford, United Kingdom).

Establishment of a Peptide-Specific T Cell Line.

1×10⁷ PBMCs were stimulated with synthetic peptide and cultured for a week. They were subsequently restimulated overnight, in the presence of anti-CD107a-FITC, washed and labelled with anti-CD137-PE (Miltenyi Biotech, Bergisch Gladbach, Germany), and counterstained with anti-CD8-APC (Biolegend). Cells were sorted using a FACS Aria cell sorter (BD Bioscience), collected, and expanded using the rapid expansion protocol previously described (Dudley et al., 2003). The T cell line was subsequently reassessed using a similar protocol.

Europium Release Killing Assay.

The DELFIA® EuTDA cytotoxicity assay (Perkin Elmer, Coventry, United Kingdom) was used according to the manufacturer's instructions. Briefly, autologous transformed B cell lines were used as target cells. These were washed and resuspended at 1×10⁶ cells/mL in RPMI/10% fetal calf serum (FCS), the relevant peptide antigen was added at 10 μg/mL, and the mixture was incubated at 37° C., 5% CO₂ in a humidified environment for 40 minutes. Subsequently, 2.5 μL/mL of the BATDA fluorescence enhancing ligand (Perkin Elmer, Coventry, United Kingdom) was added and the cells were incubated for a further 20 minutes. Cells were then washed 5× in excess medium. Target cells (1×10⁴) were added to each well of a V-bottomed 96-well plate. T-cells at varying effector to target (E:T) ratios were added to the test wells. All well volumes were made up to 200 μL. The plate was incubated for 2 hours at 37° C., 20 μL of each supernatant was transferred to a flat bottomed, white, 96 well plate, and 200 μL of Europium solution was added. This was incubated for 15 minutes with shaking at room temperature. Fluorescence was measured with a time-resolved fluorometer (Tecan INFINITE® 200 PRO; Tecan, Switzerland).

### Example 1

### Nano-Flow RP-HPLC Chromatography to Identify O-GlcNAcylated Peptides

Nano-flow RP-HPLC was the first of three experimental approaches that was employed for detecting and sequencing of O-GlcNAcylated peptides in the complex mixture of non-glycosylated peptides presented by HLA B*0702 class I MHC molecules on the surface of primary leukemia samples and cell lines. In the initial experiments, peptides from 1×10⁷ cell equivalents were fractionated by nano-flow RP-HPLC chromatography interfaced to an Orbitrap Velos mass spectrometer (Thermo Fisher Scientific, Waltham, Mass., United States of America) equipped with a front-end electron transfer dissociation (FETD) ion source (Earley et al., 2013). Collision activated dissociation (CAD) and electron transfer dissociation ETD (Syka et al., 2004) mass spectra were recorded on the top 5 most abundant peptides that eluted in a particular time window and high energy collision induced dissociation (HCD) mass spectra were recorded in a second experiment to confirm the sequences assigned to particular peptides.

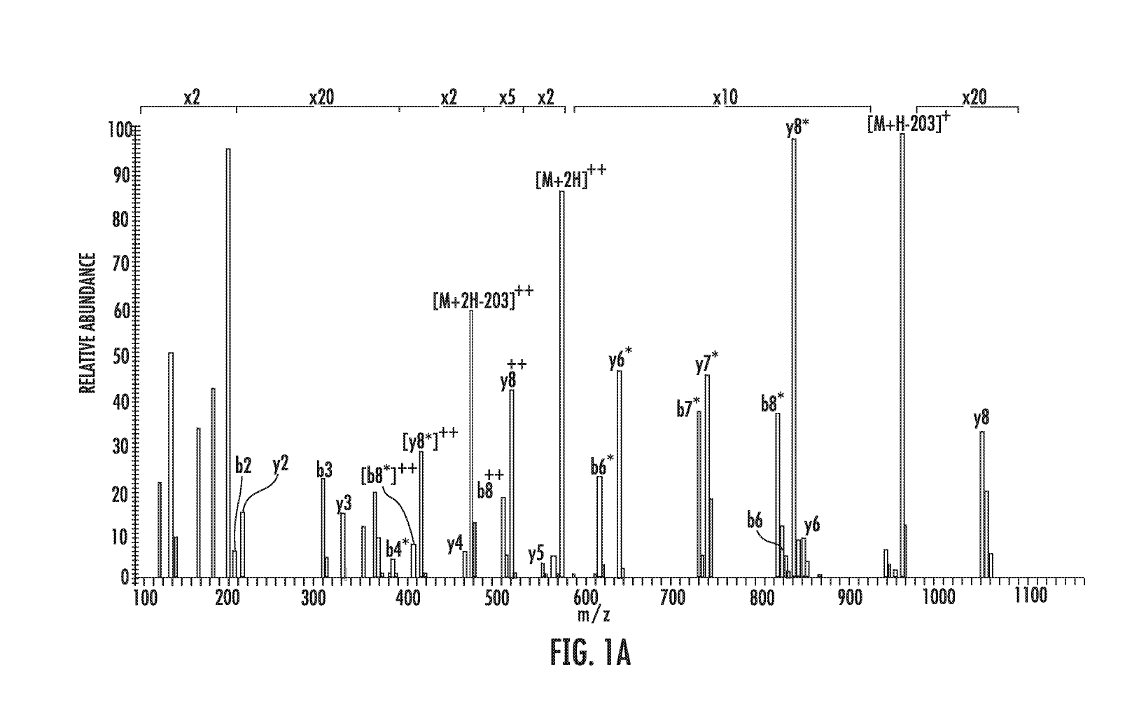

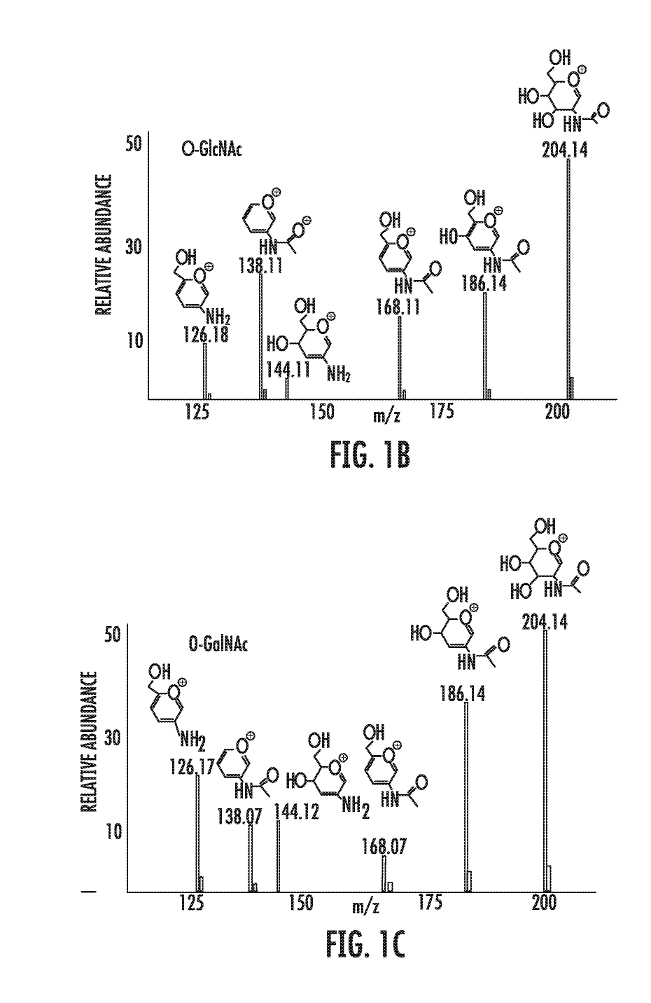

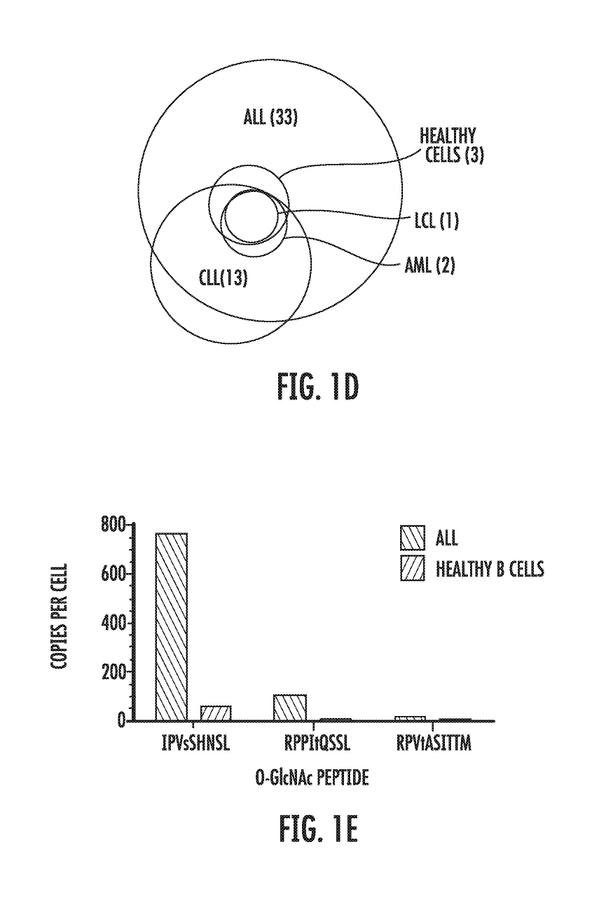

The first O-GlcNAcylated class I MHC peptide was detected during analysis of HLA B*0702 peptides presented on acute lymphocytic leukemia (ALL; see PCT International Patent Application Serial No. PCT/US2013/058477; which published as PCT International Patent Application Publication No. WO 2015/034519) using higher energy collision induced dissociation (HCD) mass spectrometry (MS) to visualize the loss of a dehydro-N-acetyl-glucosamine moiety (203Th) from fragment ions. The HCD mass spectrum of the first O-GlcNAcylated class I MHC peptide, XPVsSHNSX (SEQ ID NO: 101, where X=I or L), detected during analysis of HLA B*07:02 peptides presented on ALL is shown in FIG. 1A. Note that the experimental conditions for HCD allow initially formed fragment ions to undergo further fragmentation on subsequent collisions with nitrogen gas. As a result, the usual fragment ions of type b and y that contain an O-GlcNAc moiety can undergo loss of a dehydro-N-acetyl-glucosamine moiety (203Th). Fragments of this type are indicated by asterisks in FIG. 1A. The observed mass separation between b8 and the doubly protonated intact molecule defines the mass of y1 as that expected for X (I or L in SEQ ID NO: 101). Ions of type y at m/z 219, 333, 470 and 557 identify the last 5 residues as SHNSX (SEQ ID NO: 102). Signals for y6-y8 appear as doublets separated by 203Th and define residues 1-4 as XPVs (SEQ ID NO: 103) where the small s is O-GlcNAcylated Ser. The amino acid sequence, XPVsSHNSX (SEQ ID NO: 101), is uniquely present as IPVSSHNSL (SEQ ID NO: 19) in a single human protein, myocyte-specific enhancer factor 2C, that functions as a transcription enhancer factor required for bone marrow B-lymphopoiesis plus B-cell survival and proliferation (Canté-Barrett et al., 2014). The current approach, however, was limited by the ability of non-glycosylated peptides to suppress electrospray ionization of co-eluting O-GlcNAcylated peptides (Kastrup et al., 2000; Wang et al., 2010a)

### Example 2

### Enrichment of O-GlcNAcylated Peptides by Selective Esterification

A second experimental approach for detection and characterization of 0-GlcNAcylated peptides at the attomole level was developed to overcome the known ability of non-glycosylated peptides to suppress electrospray ionization of co-eluting O-GlcNAcyated peptides (Kastrup et al., 2000; Wang et al., 2010a; Wang et al., 2010b). Enrichment of O-GlcNAcylated peptides from the complex mixture of class I MHC peptides expressed on ALL was accomplished by selective esterification of the glycoside unit with aminophenylboronic acid linked to POROS® AL 20 beads (Thermo Fisher Scientific, Waltham, Mass., United States of America) under anhydrous conditions in dimethylformamide. O-GlcNAcylated peptides were then released in 0.1% acetic acid and analyzed by an instrument method that automatically recorded electron transfer dissociation (ETD) spectra whenever the CAD spectrum contained fragment ions corresponding to the loss of dehydro-N-acetyl-glucosamine (203Th). See Zhao et al., 2011. The same protocol can also be used to trigger the instrument to record a high energy collision induced dissociation (HCD) spectrum. It is believed that this was the first O-GlcNAc enrichment procedure to achieve quantitative yields from femtomoles of starting material, which could be critical for efficient identification of these antigens from patient samples.

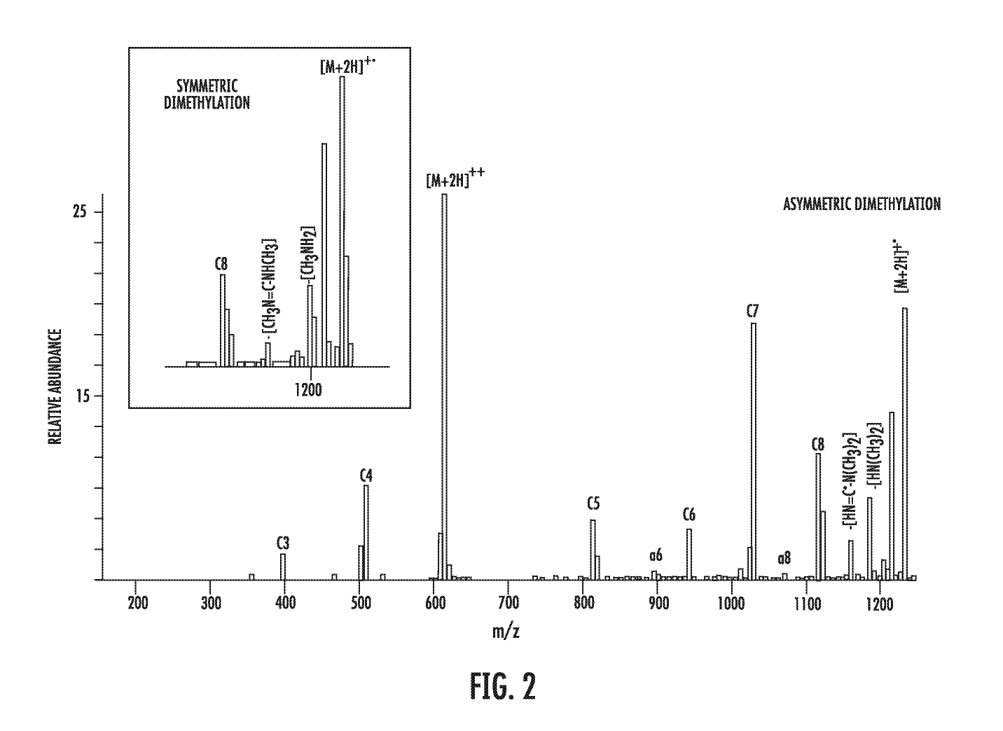

One of the new peptides detected with this protocol contained two different post-translational modifications: dimethylated Arg and an O-GlcNAcylated-Thr (see PCT International Patent Application Publication No. WO 2015/034519). The ETD spectrum for this peptide (FIG. 2) shows ions of type c (c₃-c₈) that define the last five amino acids as XtQSSX (SEQ ID NO: 99), where the small t is O-GlcNAcylated-Thr and X is either Leu or Ile. The CAD spectrum of this peptide contains an abundant ion corresponding to y8 at m/z 1045, so the dimethylated-Arg residue is at the n-terminus and the missing mass (194Th) corresponds to two Pro residues. The sequence RPPXtQSSX (SEQ ID NO: 100) is uniquely found in RNA binding protein 27 as RPPITQSSL (SEQ ID NO: 29). Non-methylated and mono-methylated forms of this peptide were also detected and sequenced. Assignment of the two methyl groups on Arg as either symmetrical or asymmetrical was achieved by synthesizing the two possible structures and then recording ETD spectra on both. The insert to FIG. 2 shows that the symmetric structure accepts an electron into the protonated side chain of Arg and loses both CH3N=C^{●}-NHCH₃ (71 Th) and CH₃NH₂ (31 Th), whereas the asymmetric structure accepts an electron and loses both HN═C^{●}-N(CH₃)₂ (71 Th) and HN(CH₃)₂ (45 Th). It was concluded that the RPPItQSSL (SEQ ID NO: 29) peptide is asymmetrically dimethylated on the side chain of the n-terminal Arg residue.

### Example 3

### O-GlcNAcylated Peptide Isolation Using the Orbitrap Fusion Tribrid

The third experimental approach employed for detection and sequence analysis of O-GlcNAcylated class I MHC peptides extended the sensitivity of the method and allowed for spectral information to be obtained from extremely low level peptide species. It was achieved using an instrument method that triggered an ETD spectrum whenever three of six O-GlcNAc fingerprint ions (m/z 204, 186, 168, 144, 138, and 126) were detected at >5% relative abundance in a particular HCD spectrum when the Orbitrap Fusion Tribrid recorded HCD spectra at top speed. All of these fingerprint ions in FIGS. 1B and 1C resulted because the O-GlcNAc oxonium ion at m/z 204 underwent further fragmentation as a result of multiple collisions with the background nitrogen gas in the collision chamber. The use of multiple fingerprint fragments created a reliable trigger which minimized false positives. The Orbitrap Fusion Tribrid recorded HCD spectra at top speed (>than 10 times faster than is possible on the Orbitrap Velos).

Approach three allowed for the expansion of the list of O-GlcNAcylated class I MHC peptides presented on leukemia cells and also to detect class I MHC peptides that contained disaccharide units attached to Ser, Thr, or Asn residues. The O-GlcNAcylated peptides that contained disaccharide units attached to Ser, Thr, or Asn residues are presented in Tables 4 and 5 (see also the notes following Tables 4 and 5).

These findings suggested that some of the observed glycosylated peptides might be derived from degradation of O- and N-linked glycans synthesized in the Golgi and ER rather than by reaction with O-GlcNAc transferase in the cytoplasm or nucleus. Note that the first two hexose units in N-linked glycans are usually 2-GlcNAcs. Those for O-linked glycans are usually either O-GalNAc and GlcNAc or O-GalNAc and galactose (Wolfert & Boons, 2013).

### Example 4

### 36 O-GlcNAcylated Peptides Identified on HLA B*07:02 Leukemia Samples

In total, using the three experimental approaches disclosed herein, 36 O-GlcNAcylated peptides were identified from leukemia samples (see Table 7). 92% (33/36) of the O-GlcNAcs identified were only found on the leukemia samples tested and not the healthy tissue samples, making them potential leukemia neoantigens (see Table 7 and FIG. 1D). Just under a quarter ( 7/32) of the proteins that the 0-GlcNAcylated peptides derived from were associated with key cancer pathways (as defined by the NCI pathway interaction database). These pathways included many classical cancer signaling pathways, involving genes such as p38, p53, c-Myc, Notch, Wnt, Rb, ErbB1 and MAPK. Of note, IPVsSHNSL (SEQ ID NO: 19), which derives from Mef2c, a transcription factor implicated in leukemogenesis (Canté-Barrett et al., 2014), was identified on nearly all of the leukemia samples tested (1/1 ALL, 3/3 CLL, 1/1 AML, 1/1 HCL), and while it could be detected on healthy B cells, was present at far lower levels (see FIG. 1E). An incidental, but significant, finding was that one of the novel O-GlcNAcylated peptides detected, RPPItQSSL (SEQ ID NO: 29), contained another post-translational modification: a methylated Arg residue at position 1. Not only that, but the same peptide was also found with an asymmetrically dimethylated Arg residue (see FIG. 2).

### Example 5

### Differentiating O-GlcNAc-Containing and O-GalNAc-Containing Peptides

Since O-GlcNAc and O-GalNAc are isobaric but have different biological properties, it was important to confirm that the peptides identified indeed contained O-GlcNAc modifications and not O-GalNAc modifications. Furthermore, since some MHC class I peptides identified contained disaccharide units (see the Notes following Tables 4 and 5), they might have been derived from degradation of O- and N-linked glycans synthesized in the Golgi and ER, rather than true cancer neoantigens.

To validate that the peptide antigens tested were O-GlcNAcylated, an in vitro enzyme reaction was utilized. β1-4-galactosyltransferase (GalT1) was shown to transfer N-azidoacetylgalactosamine (GalNAz) to four peptides (IPVsSHNSL (SEQ ID NO: 19) and (me-)RPPItQSSL (SEQ ID NO: 29)) in the ALL sample. Additionally, it was determined that synthetic O-GlcNAcylated vs. O-GalNAcylated peptides could be differentiated based on the relative ion abundances observed for fragments derived from the oxonium ion at m/z 204 in the corresponding fingerprint region of the HCD mass spectra (Zhao et al., 2011; see also FIGS. 1B and 1C). All of the peptides observed (see Table 7) produced HCD spectra with the necessary fingerprint region to confirm their identity as O-GlcNAc peptides.

### Example 6

### Identification of Disaccharide-Modified Peptides

Ten of the peptides detected were also found with disaccharide units attached to the same residues that were O-GlcNAcylated. It was determined that these corresponded to a hexose bound to a hexNAc since the oxonium ion observed for all of these peptides occurs at m/z 366 (204+162). This was likely the result of the transfer of galactose to the O-GlcNAcylated peptide by a β-N-acetylglucosamine β1-4 galactosyltransferase; however, the remote possibility that this instead could have involved the O-glycan synthetic pathway, where the first residue to be added was a GalNAc and the second was either galactose or GlcNAc, needed to be excluded. Again, using synthetic peptides (IPVsSHNSL (SEQ ID NO: 19) modified with Gal-GalNAc and Gal-GlcNAc) the fingerprint patterns for fragmentation of the oxonium ion at m/z 204 in HCD mass spectra could be distinguished, confirming that none of the disaccharide modified peptides in Table 7 were derived from the O-glycan synthetic pathway.

Two of the glycosylated peptides in Table 7, APRGnVTSL (SEQ ID NO: 3) and KPTLYnVSL (SEQ ID NO: 22), had disaccharide units, hexose-hexNAc, attached to Asn residues. Both peptides had consensus sequences, NX(S/T) for attachment of N-linked oligosaccharides. Thus, the observed hexose-GlcNAc disaccharide units attached to Asn in these peptides probably resulted from degradation of the N-linked oligosaccharide structures to a single N-linked GlcNAc that then accepted a hexose such as galactose (from a β-N-acetylglucosamine β1-4 galactosyl-transferase). This was a new finding as the enzyme N-glycanase 1 is responsible for removing all N-linked glycosylation prior to loading onto MHC class I molecules, potentially suggesting a new source of neoantigens in leukemia (Suzuki et al., 2016).

### Example 7

### Leukemia-Associated Glycopeptides Elicited Potent Memory T Cell Responses in Healthy Donors

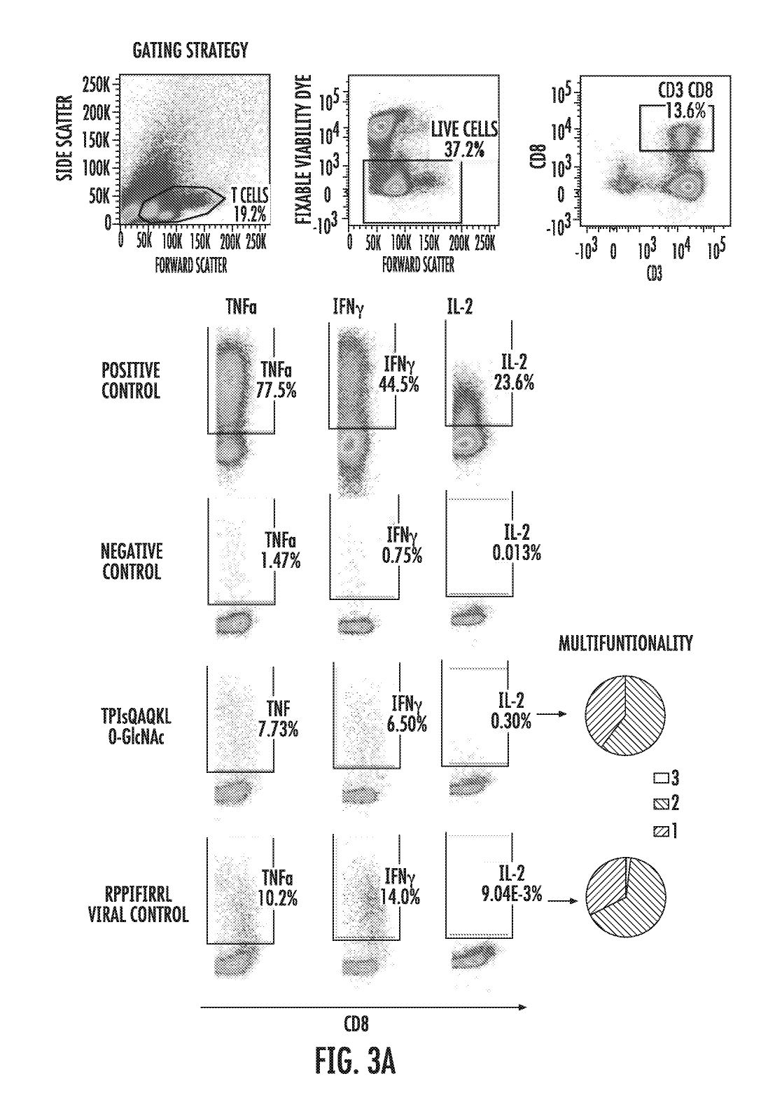

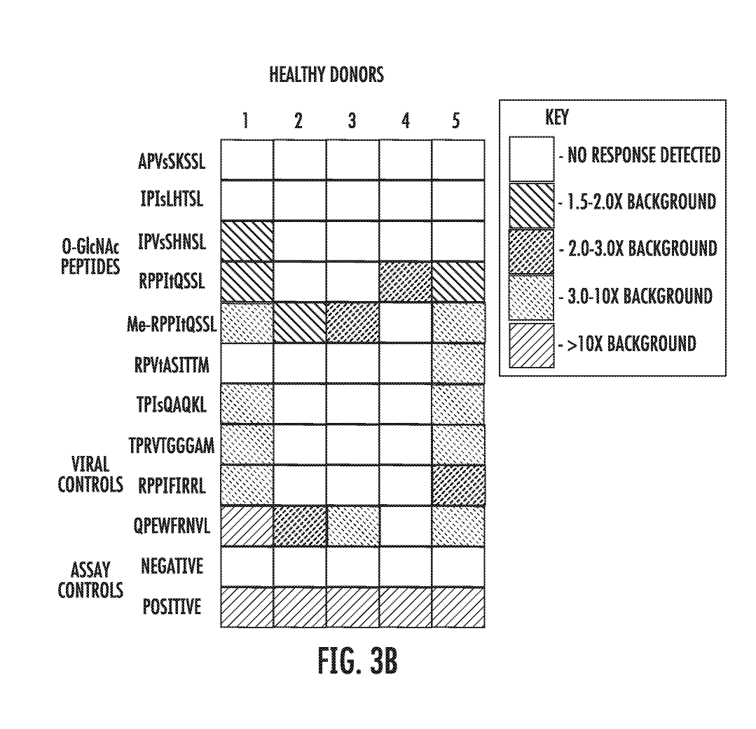

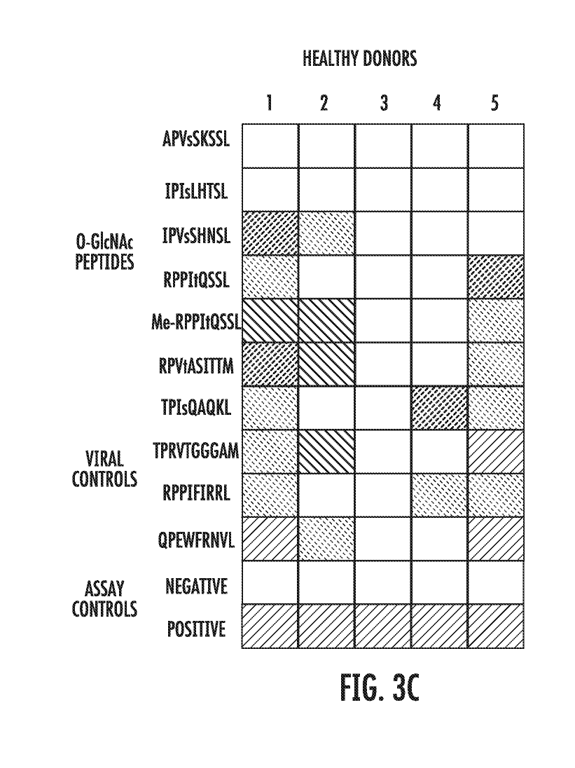

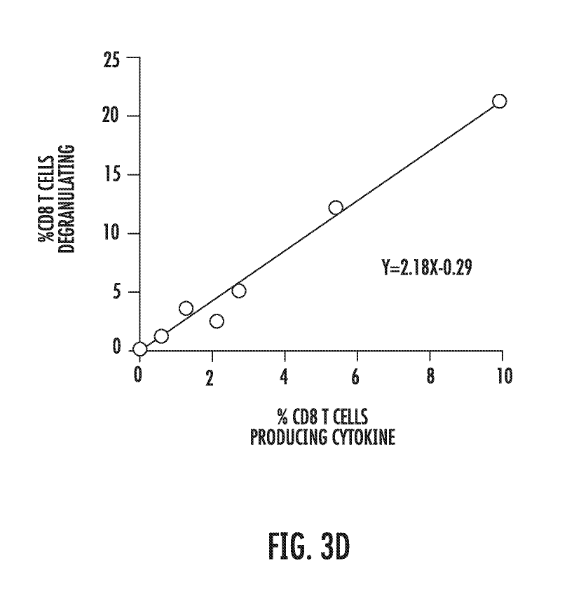

Previous studies have highlighted how post-translationally modified antigens can be immunogenic, with immunity against leukemia-associated MHC class I phosphopeptides having been shown to be present in healthy individuals. Immunity against naturally processed MHC class-I O-GlcNAc or methylated peptides has not been studied, but it was hypothesized that it may exist in healthy individuals. Immunogenicity in HDs was assessed using seven of the O-GlcNAcylated peptides discovered on leukemic cells (see FIG. 3). Employing the gating strategy depicted in FIG. 3A, five of the seven (71%) HLA-B*0702 glycopeptides were found to be immunogenic-heterogeneous responses were seen, with both intra- and inter-donor variation (FIGS. 3B and 3C). Importantly, all HDs had immunity to at least one of the glycopeptides and two had strong responses, similar to the magnitude of responses against chronic viral antigens. Degranulation was assessed as a proxy for killing (FIG. 3C) and despite some background staining, degranulation significantly correlated with multifunctional cytokine responses (FIG. 3D), suggesting that these T cells targeting O-GlcNAcylated peptide antigens had a cytotoxic phenotype.

### Example 8

### T Cells Targeting a Methylated Glycopeptide Specifically Targeted the Modifications and Killed

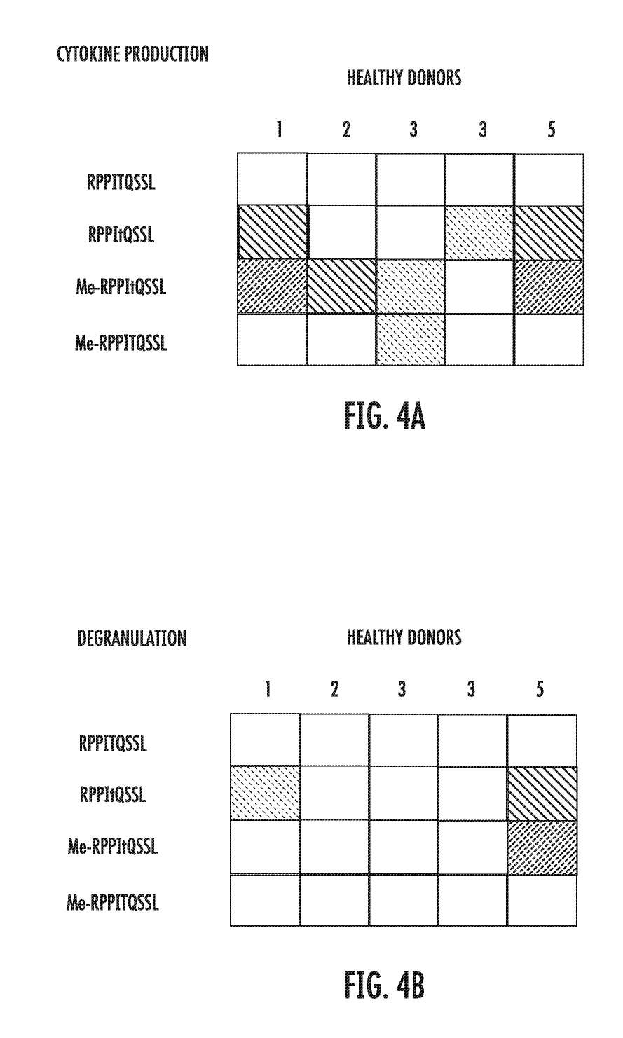

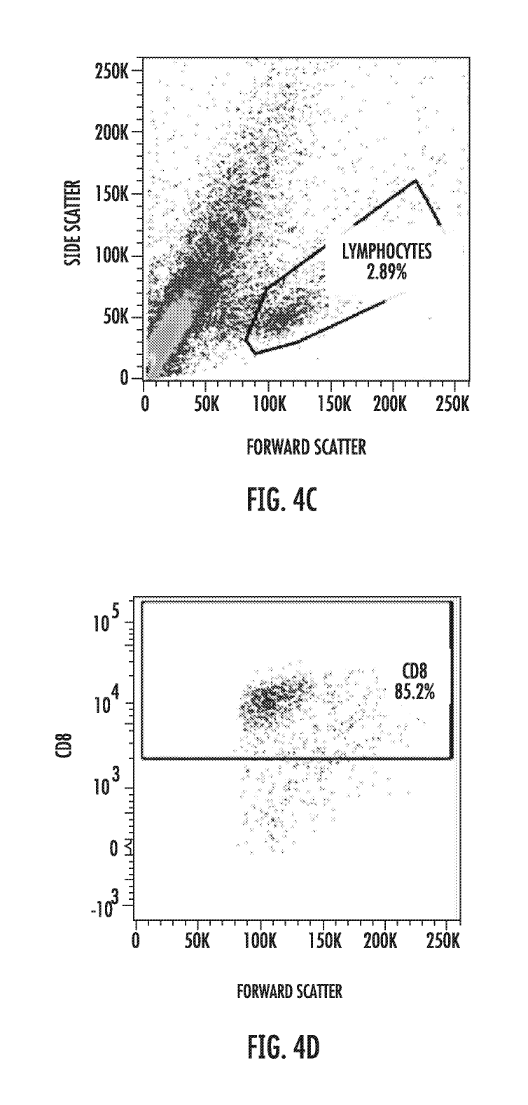

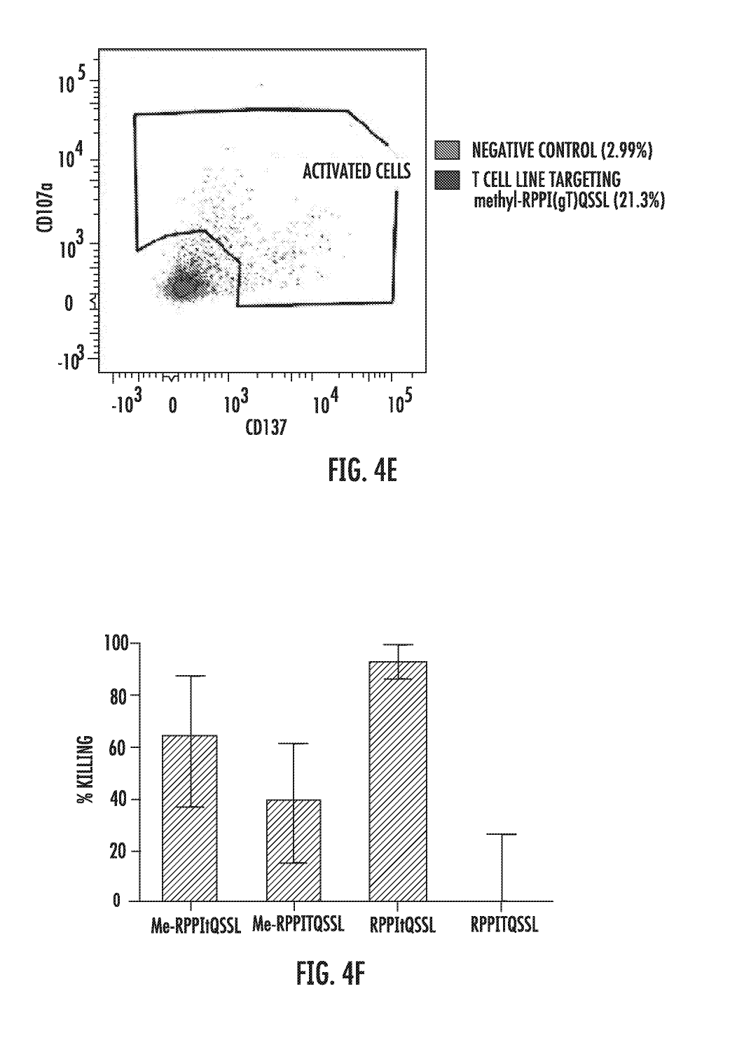

As responses were seen against the intriguing methylated glycopeptide ((me-R)PPI(GlcNAc-T)QSSL; SEQ ID NO: 29) in 4/5 (80%) of HDs tested, two being potent, these responses were further analyzed using peptides that were either methylated or glycosylated. While no T cell responses were seen against the unmodified peptide, responses in different individuals were seen targeting either the glycosylated or the methylated peptide (see FIGS. 4A and 4B). In the two donors with potent responses to the methylated glycopeptides, there were fewer T cells recognizing the glycopeptide alone, suggesting that the methylation could somehow increase immunogenicity. To assess this further, a T cell line was initiated using the methylated glycopeptide. After culture, around 18% of T cells were shown to be specific for the methylated glycopeptide (see FIGS. 4C-4E). Autologous transformed B cells were pulsed with modified and unmodified peptides and killing by the T cell line assessed. Specific killing was seen of the B cells pulsed with methylated, O-GlcNAcylated, and doubly modified peptide, but not with the unmodified peptide (see FIG. 4F). These results suggested that the modified peptides targeted by the endogenous anti-leukemia T cell response were identified, which could lead to fruitful targets for novel immunotherapeutics.

### Discussion of the Examples

Previously, identification of cancer neoantigens has focused on mutated peptides. Here, three methodologies are outlined for the identification of MHC class I peptides containing a little-known post-translational modification (PTM), O-GlcNAc, a potential new type of cancer neoantigens. Utilizing these methods, it was possible to identify 36 GlcNAcylated peptides from primary leukemia samples that are presented by MHC Class I HLA B*0702, and showed that a memory T cell response against a subset of these antigens could be found in HDs. Furthermore, peptides that contained other moieties not previously seen on MHC class I peptides from cancer samples—namely methyl, disaccharide, and N-linked GlcNAc groups—were also identified. Peptides containing these PTMs offer a hitherto untapped source of neoantigens in leukemia.

These neoantigens created by PTMs can be found on leukemic cells because of their aberrant cell signaling. This has been reported for phosphopeptide leukemia antigens and O-GlcNAcylation sites are usually identical, or in close proximity, to those that get phosphorylated (Wells et al., 2004; Cobbold et al., 2013). Furthermore, aberrant O-GlcNAcylation has been shown to correlate with augmented cancer cell proliferation, survival, invasion, and metastasis (de Queiroz et al., 2014). The essential nature of these pathways to the leukemic cells suggests that these PTM neoantigens may not be patient specific, as seen with the mutated neoantigens, but common across patients of the same HLA-type (Rizvi et al., 2015; McGranahan et al., 2016) (5, 22). Indeed, many of them were identified on multiple samples from leukemia patients, even those with different clinical types (see FIG. 1D). Antigens from these key signaling pathways are ideal targets for immunotherapies since the leukemic cell is unlikely to be able to survive without these pathways, reducing the risk of immune escape. As such, they may provide an attractive new avenue for immunotherapeutic targeting.

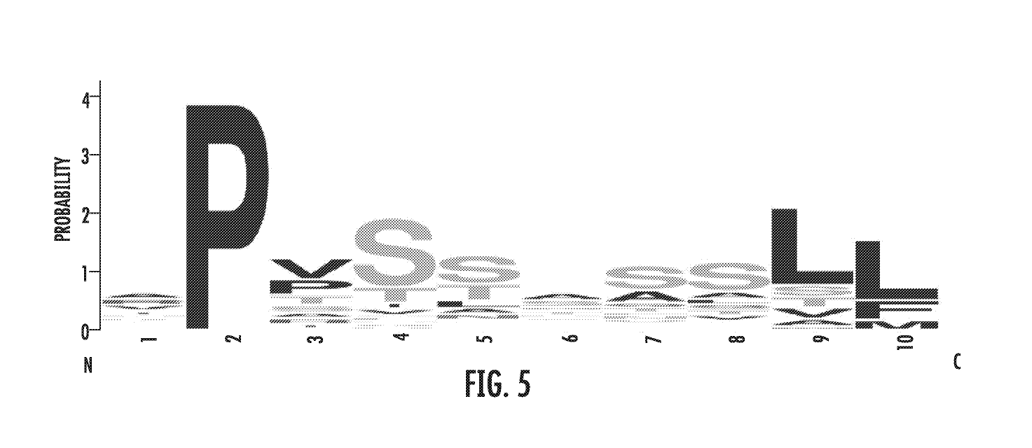

Not only are these neoantigens present on leukemia samples, but positional analysis indicates that the GlcNAc residues may be optimally positioned for T cell recognition. The GlcNAc group is in the middle of the peptide (up to 34/36; 62% P4, 18% P5, 21% equivocal P4/P5; see FIG. 5), identical to the preferred position of phosphate groups in phosphopeptides and where structural studies have revealed that the CDR3 regions of the TCR loops around the center of the peptide (Mohammed et al., 2008). Indeed, previous structural studies in mouse of TCR binding have demonstrated that GlcNAc modified antigens are recognized in this manner (Glithero et al., 1999).

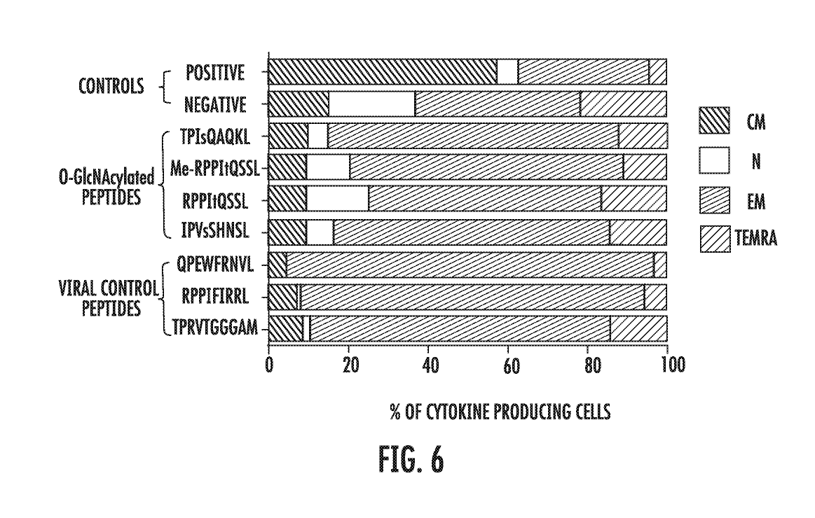

Potent multifunctional memory T cell responses were seen against these 0-GlcNAcylated leukemia antigens in HDs, suggesting that these neoantigens may reflect on endogenous immunosurveillance systems against leukemia (see FIGS. 3 and 6). Not only did HD T cells recognize the PTM neoantigen, but they were also shown to specifically kill cells presenting modified peptides (see FIG. 4F). The most immunogenic peptide identified was me-RPPItQSSL (SEQ ID NO: 29), containing both a methylated arginine and O-GlcNAcylated serine. It is tempting to speculate that combined modifications lead to the most dramatic structural change and, therefore, the most antigenically distinct from self-peptides. That T cells may recognize and kill cells presenting this peptide with either the methylation, or the O-GlcNAc modification, but not the unmodified peptide, has also been shown. This potent antigen, targetable by T cells from several HDs, is a very attractive target for the development of novel immunotherapeutics.

Overall, the presently disclosed subject matter relates to both glycosylated and methylated residues as a new class of tumor antigens broadening the availability of immunotherapy targets and may yield safe and effective therapeutics for leukemia.

TABLE 3
O-GlcNAcylated Class I MHC Peptides on Cancer
Associated with HLA A*0201^{a}

| SEQ ID NO | Sequence | Start Stop | UniProt | Source | Protein |
|---|---|---|---|---|---|
| 1 | ALTtSAHSV | 592 | 600 | Q9H422 | Homeodomain-interacting |
| | | | | | protein kinase 3 |
| 1 | ALTTsAHSV | 592 | 600 | Q9H422 | Homeodomain-interacting |
| | | | | | protein kinase 3 |
| 1 | ALTtsAHSV | 592 | 600 | Q9H422 | Homeodomain-interacting |
| | | | | | protein kinase 3 |
| 10 | GLSsLAEEAA | 20 | 29 | Q9BYD3 | 39S ribosomal protein L4, |
| | | | | | mitochondrial |
| 38 | VLTsNVQTI | 507 | 515 | P32519 | ETS-related transcription |
| | | | | | factor Elf-1 |

^{a}Glycosylated peptides where s ant t are Ser and Thr residues modified with an
O-GlcNAc moiety

TABLE 4
O-GlcNAcylated Class I MHC Peptides on
Cancer Associated with HLA B*0702^{b}

| SEQ ID NO | Sequence | Start | Stop | UniProt | Source Protein |
|---|---|---|---|---|---|
| 2ⁱ | APPsTSAAAL | 405 | 414 | Q86TM6 | E3 Ubiquitin-protein |
| | | | | | ligase synoviolin |
| 2ⁱ | APPStSAAAL | 405 | 414 | Q86TM6 | E3 Ubiquitin-protein |
| | | | | | ligase synoviolin |
| 2ⁱ | APPSTsAAAL | 405 | 414 | Q86TM6 | E3 Ubiquitin-protein |
| | | | | | ligase synoviolin |
| 2ⁱ | APPstSAAAL | 405 | 414 | Q86TM6 | E3 Ubiquitin-protein |
| | | | | | ligase synoviolin |
| 2ⁱ | APPStsAAAL | 405 | 414 | Q86TM6 | E3 Ubiquitin-protein |
| | | | | | ligase synoviolin |
| 4 | APRtNGVAM | 187 | 195 | Q92567 | Protein FAM168A |
| 5 | APTsAAAL | 1116 | 1123 | Q86Z02 | Homeodomain- |
| | | | | | interacting protein |
| | | | | | kinase 1 |
| 6 | APTsASNVM | 238 | 246 | P28323; | ETS-related transcription |
| | | | | P28324 | factor Elk-4 |
| 6 | APTSAsNVM | 238 | 246 | P28323; | ETS-related transcription |
| | | | | P28324 | factor Elk-4 |
| 7 | APVsASASV | 1807 | 1815 | Q9Y520 | Protein PRRC2C |
| 8 | APVsSKSSL | 850 | 858 | Q86Z02 | Homeodomain- |
| | | | | | interacting protein |
| | | | | | kinase 1 |
| 9 | EPsSTVVSL | 1077 | 1085 | O75129 | Astrotactin-2 |
| 9 | EPSsTVVSL | 1077 | 1085 | O75129 | Astrotactin-2 |
| 9 | EPSStVVSL | 1077 | 1085 | O75129 | Astrotactin-2 |
| 11 | HPMsTASQV | 345 | 353 | Q8N6B4 | Myeloid/lymphoid or |
| | | | | | mixed-lineage leukemia |
| 12ⁱⁱⁱ | HPsStAAVL1 | 740 | 748 | Q86XN7 | Proline and serine-rich |
| | | | | | protein 1 |
| 13ⁱⁱⁱ | HPSstAAVL | 740 | 748 | Q86XN7 | Proline and serine-rich |
| | | | | | protein 1 |
| 13ⁱⁱⁱ | HPssTAAVL | 740 | 748 | Q86XN7 | Proline and serine-rich |
| | | | | | protein 1 |
| 14 | HPsSTASTAL | 3041 | 3050 | Q96T58 | Msx2-interacting protein |
| 14 | HPSsTASTAL | 3041 | 3050 | Q96T58 | Msx2-interacting protein |
| 14 | HPSStASTAL | 3041 | 3050 | Q96T58 | Msx2-interacting protein |
| 16 | IPIsLHTSL | 1959 | 1967 | Q5JSZ5 | Protein PRRC2B |
| 17 | IPTsSVLSL | 710 | 718 | 015027 | Protein transport protein |
| | | | | | Sec16A |
| 18^{iv} | IPVsKPLSL | 104 | 112 | Q16621 | Leucine zipper protein 1 |
| 19^{v} | IPVsSHNSL | 147 | 155 | Q06413 | Myocyte-specific |
| | | | | | enhancer factor 2C |
| 19^{v} | IPVssHNSL | 147 | 155 | Q06413 | Myocyte-specific |
| | | | | | enhancer factor 2C |
| 20^{vi} | KPPTsQSSVL | 411 | 420 | Q5T6F2 | Ubiquitin associated |
| | | | | | protein 2 |
| 20^{vi} | KPPtSQSSVL | 411 | 420 | Q5T6F2 | Ubiquitin associated |
| | | | | | protein 2 |
| 23 | LPRNstMM | 335 | 342 | Q9NPI6 | mRNA-decapping |
| | | | | | enzyme 1A |
| 24 | LPTsLPSSL | 2464 | 2472 | P46531 | Neurogenic locus notch |
| | | | | | homolog protein 1 |
| 25^{ix} | NIPVRPTtNTF | 218 | 227 | Q7Z3K3 | Pogo transposable |
| | | | | | element with ZNF |
| | | | | | domain |
| 27 | NPVsLPSL | 831 | 838 | Q6VMQ6 | Activating transcription |
| | | | | | factor 7-interacting |
| | | | | | protein 1 |
| 29^{xi} | RPPItQSSL | 382 | 390 | Q9P2N5 | RNA binding protein 27 |
| 29^{xi} | (Me)RPPItQSSL | 382 | 390 | Q9P2N5 | RNA binding protein 27 |
| 29^{xi} | (diMe)RPPItQSSL | 382 | 390 | Q9P2N5 | RNA binding protein 27 |
| 31 | RPPsSSQQL | 1758 | 1766 | Q8WYB5 | Histone acetyltransferase |
| | | | | | KAT6B |
| 31 | RPPSsSQQL | 1758 | 1766 | Q8WYB5 | Histone acetyltransferase |
| | | | | | KAT6B |
| 31 | RPPSSsQQL | 1758 | 1766 | Q8WYB5 | Histone acetyltransferase |
| | | | | | KAT6B |
| 32 | RPPVtKASSF | 341 | 350 | Q9Y2K5 | R3H domain containing |
| | | | | | protein 2 |
| 33 | RPVtASITTM | 927 | 936 | Q9ULH7 | MKL/myocardin-like |
| | | | | | protein 2 |
| 34 | TPASsRAQTL | 2320 | 2329 | Q01082 | Spectrin beta chain, non- |
| | | | | | erythrocytic 1 |
| 35 | TPAsSSSAL | 875 | 883 | Q9NPG3 | Ubinucleain 1 |
| 36 | TPIsQAQKL | 3024 | 3032 | Q96L91 | E1A-binding protein |
| | | | | | p400 |
| 39 | VPAsSTSTL | 576 | 584 | Q9NYV4 | Cyclin dependent kinase 13 |
| 41 | VPtTSSSL | 1284 | 1291 | Q14004 | Cyclin dependent kinase 13 |
| 41 | VPTtSSSL | 1284 | 1291 | Q14004 | Cyclin dependent kinase 13 |
| 41 | VPTTsSSL | 1284 | 1291 | Q14004 | Cyclin dependent kinase 13 |
| 42 | VPVsGTQGL | 93 | 101 | P23511 | Nuclear transcription |
| | | | | | factor Y subunit alpha |
| 43 | VPVsNQSSL | 146 | 154 | Q14814 | Myocyte-specific |
| | | | | | enhancer factor 2D |
| 44 | VPVsSASEL | 595 | 603 | C9J6P4 | Zinc finger CCCH-type, |
| | | | | | antiviral 1 |
| 45 | VPVsVGPSL | 1156 | 1164 | Q86Z02 | Homeodomain-interacting |
| | | | | | protein kinase 1 |

^{b}Glycosylated peptides where s and t are Ser and Thr residues modified with an
O-GlcNAc moiety
ⁱPeptide was detected in a total of five forms: single GlcNAc, double GlcNAc, single
hexose-GlcNAc, single GlcNAc (S6) + hexose-GlcNAc (T5), and double hexose-GlcNAc
ⁱⁱⁱTwo GlcNAcs were detected, but could not be assigned to specific amino acids.
^{iv}Peptide was detected in two forms: GlcNAc (S4) and hexose-GlcNAc (S4)
^{v}Peptide was detected in four forms: GlcNAc (S4), double GlcNAc (S4, S5), single
hexose-GlcNAc (S4), and an acetyl-GlcNAc (S4)
^{vi}Peptide was detected in two forms: GlcNAc and hexose-GlcNAc (T4)
^{ix}Peptide was detected in two forms: hexose-GlcNAc and asymmetric di-methyl (R4) +
hexose-GlcNAc (T7)
^{xi}Peptide was detected in four forms: GlcNAc (T5), mono-methyl (R1) + GlcNAc (T5),
asymmetric di-methyl (R1) + GlcNAc (T5), and asymmetric di-methyl (R1) + acetyl-
GlcNAc (T5)

TABLE 5
Hexose-GlcNAcylated Class I MHC Peptides on
Cancer Associated with HLA B*0702_{c}

| SEQ ID NO | Sequence | Start | Stop | UniProt | Source Protein |
|---|---|---|---|---|---|
| 2ⁱ | APPSTsAAAL | 405 | 414 | Q86TM6 | E3 Ubiquitin- |
| | (hexose-GlcNAc) | | | | protein ligase |
| | | | | | synoviolin |
| 2ⁱ | APPsTSAAAL | 405 | 414 | Q86TM6 | E3 Ubiquitin- |
| | (hexose-GlcNAc) | | | | protein ligase |
| | | | | | synoviolin |
| 2ⁱ | APPStSAAAL | 405 | 414 | Q86TM6 | E3 Ubiquitin- |
| | (hexose-GlcNAc) | | | | protein ligase |
| | | | | | synoviolin |
| 2ⁱ | APPStsAAAL | 405 | 414 | Q86TM6 | E3 Ubiquitin- |
| | (hexose-GlcNAc, | | | | protein ligase |
| | GlcNAc) | | | | synoviolin |
| 2ⁱ | APPStsAAAL | 405 | 414 | Q86TM6 | E3 Ubiquitin- |
| | (2 hexose-GlcNAc) | | | | protein ligase |
| | | | | | synoviolin |
| 2ⁱ | APPsTsAAAL | 405 | 414 | Q86TM6 | E3 Ubiquitin- |
| | (2 hexose-GlcNAc) | | | | protein ligase |
| | | | | | synoviolin |
| 2ⁱ | APPstSAAAL | 405 | 414 | Q86TM6 | E3 Ubiquitin- |
| | (2 hexose-GlcNAc) | | | | protein ligase |
| | | | | | synoviolin |
| 3ⁱⁱ | APRGnVTSL | 60 | 68 | Q9NR96 | Toll-like receptor 9 |
| | (hexose-GlcNAc) | | | | |
| 18^{iv} | IPVsKPLSL | 104 | 112 | Q16621 | Leucine zipper |
| | (hexose-GlcNAc) | | | | protein 1 |
| 19^{v} | IPVsSHNSL | 147 | 155 | Q06413 | Myocyte-specific |
| | (hexose-GlcNAc) | | | | enhancer factor |
| | | | | | 2C |
| 20^{vi} | KPPTsQSSVL | 411 | 420 | Q5T6F2 | Ubiquitin |
| | (hexose-GlcNAc) | | | | associated protein 2 |
| 21^{vii} | KPPVsFFSL | 95 | 103 | Q6PKC3 | Thioredoxin |
| | (hexose-GlcNAc) | | | | domain containing |
| | | | | | protein 11 |
| 22^{viii} | KPTLYnVSL | 373 | 381 | P04220 | Ig Mu heavy chain |
| | (hexose-GlcNAc) | | | | disease protein |
| 28^{x} | PPStSAAAL | 405 | 414 | Q86TM6 | E3 Ubiquitin- |
| | (hexose-GlcNAc) | | | | protein ligase |
| | | | | | synoviolin |
| 28^{x} | PPSTsAAAL | 405 | 414 | Q86TM6 | E3 Ubiquitin- |
| | (hexose-GlcNAc) | | | | protein ligase |
| | | | | | synoviolin |
| 30^{xii} | RPPQsSSVSL | 937 | 946 | 015027 | Protein transport |
| | (hexose-GlcNAc) | | | | protein Sec16A |

^{c}Glycosylated peptidees where s, t, and n are Ser, Thr, and Asn residues
modified by a disaccharide (hexose-GlcNAc)
ⁱPeptide was detected in a total of five forms: single GlcNAc, double GlcNAc,
single hexose-GlcNAc, single GlcNAc (S6) + hexose-GlcNAc (T5), and double
hexose-GlcNAc
ⁱⁱN5 is modified by N-linked hexose-GlcNAc
^{iv}Peptide was detected in two forms: GlcNAc (S4) and hexose-GlcNAc (S4)
^{v}Peptide was detected in four forms: GlcNAc (S4), double GlcNAc (S4, S5),
single hexose-GlcNAc (S4), and an acetyl-GlcNAc (S4)
^{vi}Peptide was detected in two forms: GlcNAc and hexose-GlcNAc (T4)
^{vii}S5 is modified by O-linked hexose-GlcNAc
^{viii}N7 is modified by N-linked hexose-GlcNAc
^{x}T4 or S5 is modified by O-linked hexose-GlcNAc
^{xii}S5 is modified by O-linked hexose-GlcNAc

TABLE 6
O-GlcNAcylated Class I MHC Peptides
on Cancer Associated with HLA B*35d

| SEQ | | | | | |
|---|---|---|---|---|---|
| ID NO | Sequence | Start | Stop | UniProt | Source Protein |
| 15 | HPTtVASY | 57 | 64 | Q05D65 | ZFR protein |
| 26 | MPVtSSSFF | 408 | 416 | Q01543 | Friend leukemia |
| | | | | | integration 1 |
| | | | | | transcription |
| | | | | | factor |
| 37 | TPVsSANMM | 344 | 352 | P68400 | Casein kinase II |
| | | | | | subunit alpha |
| 40 | VPAtHGQVTY | 306 | 315 | P48436 | Transcription |
| | | | | | factor SOX-9 |

dGlycosylated peptides where s and t are Ser and Thr
residues modified with an O-GlcNAc moiety

TABLE 7
O-GlcNAcylated Peptides Presented by HLA B*0702
Class I MHC Molecules on Leukemia

| SEQ ID NO: | Sequence | Tumor | Source Protein |
|---|---|---|---|
| 2ⁱ | APP(sts)AAAL | ALL, CLL | E3 Ubiquitin-protein ligase |
| | | | synoviolin |
| 3ⁱⁱ | APRGnVTSL | CLL | Toll-like receptor 9 |
| 4 | APRtNGVAM | ALL, CLL | Protein FAM168A |
| 5 | APTsAAAL | ALL | Homeodomain-interacting protein |
| | | | kinase 1 |
| 7 | APVsASASV | ALL | Protein PRRC2C |
| 8 | APVsSKSSL | ALL, CLL | Homeodomain-interacting protein |
| | | | kinase 1 |
| 9 | EP(sst)VVSL | ALL | Astrotactin-2 |
| 11 | HPMsTASQV | ALL | Clathrin assembly lymphoid |
| | | | myeloid leukemia |
| 12ⁱⁱⁱ | HP(sss)AAVL | ALL, CML | Proline and serine-rich protein 1 |
| 14 | HP(sst)ASTAL | ALL | Msx2-interacting protein |
| 16 | IPIsLHTSL | ALL | Protein PRRC2B |
| 17 | IPTsSVLSL | ALL | Protein transport protein Sec 16A |
| 18^{iv} | IPVsKPLSL | AML, ALL, CLL | Leucine zipper protein 1 |
| 19^{v} | IPVsSHNSL | AML, ALL, CLL, | Myocyte-specific enhancer factor |
| | | GM, JY, S, To | 2C |
| 20^{vi} | KPP(ts)QSSVL | ALL | Ubiquitin associated protein 2 |
| 21^{vii} | KPPVsFFSL | ALL | Thioredoxin domain containing |
| | | | protein 11 |
| 22^{viii} | KPTLYnVSL | CLL | Ig Mu heavy chain disease protein |
| 23 | LPRN(st)MM | ALL | mRNA-decapping enzyme 1A |
| 24 | LPTsLPSSL | ALL | Neurogenic locus notch homolog |
| | | | protein 1 |
| 25^{ix} | MPVRPTtNTF | ALL | pogo transposable element with |
| | | | ZNF domain |
| 27 | NPVsLPSL | ALL | Activating transcription factor 7- |
| | | | interacting protein |
| 28x | PPS(ts)AAAL | ALL | E3 Ubiquitin-protein ligase |
| | | | synoviolin |
| 29^{xi} | RPPItQSSL | ALL, S | RNA binding protein 27 |
| 30^{xii} | RPPQsSSVSL | ALL | Protein transport protein Sec 16A |
| 31 | RPP(sss)QQL | ALL | Histone acetyltransferase KAT6B |
| 32 | RPPVtKASSF | ALL, CLL | R3H domain containing protein 2 |
| 33 | RPVtASITTM | ALL, CLL, S | MKL/myocardin-like protein 2 |
| 34 | TPASsRAQTL | CLL | Spectrin beta chain, non- |
| | | | erythrocytic 1 |
| 35 | TPAsSSSAL | ALL, CLL | Ubinucleain 1 |
| 36 | TPIsQAQKL | ALL | E1A-binding protein p400 |
| 39 | VPAsSTSTL | ALL, CLL | Cyclin dependent kinase 12 |
| 41 | VPTtSSSL | ALL | Cyclin dependent kinase 13 |
| 42 | VPVsGTQGL | ALL | Nuclear transcription factor Y |
| | | | subunit alpha |
| 43 | VPVsNQSSL | ALL | Myocyte-specific enhancer factor 2D |
| 44 | VPVsSASEL | ALL | Zinc finger CCCH-type, antiviral 1 |
| 45 | VPVsVGPSL | ALL | Homeodomain-interacting protein |
| | | | kinase 1 |

CLL; chronic lymphocytic leukemia, AML; acute myeloid leukemia, ALL; acute lymphoblastic
leukemia, CML; chronic myeloid leukemia, J; JY cell line, G; GM030107 cell line, S;
spleen and To; tonsil. Small letters, s, t, and n specify Ser, Thr, and Asn residues
that are modified by O-GlcNAc unless otherwise indicated in Notes i-xii accompanying
Tables 4 and 5 above. Parentheses enclose s and t residues that could be a site of
GlcNAcAcylation.

### REFERENCES

All references listed in the instant disclosure, including but not limited to all patents, patent applications and publications thereof, scientific journal articles, and database entries (including but not limited to GENBANK® and UniProt biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J Mol Biol 215:403-410.
Altschul et al. (1990b) Protein database searches for multiple alignments. Proc Natl Acad Sci USA 87(14):5509-5513.
Altschul et al. (1997) Gapped BLAST and PSI-BLAST: a new generation of protein database search programs. Nucleic Acids Res 25:3389-3402.
Ansell et al. (2015) PD-1 blockade with nivolumab in relapsed or refractory Hodgkin's lymphoma. N Eng J Med 372:311-319.
Blum et al. (2013) Pathways of antigen processing. Annu Rev Immunol 31:443-473.
Brahmer et al. (2012) Safety and activity of anti-PD-L1 antibody in patients with advanced cancer. N Engl J Med 366:2455-2465.
Canté-Barrett et al. (2014) Myocyte enhancer factor 2C in hematopoiesis and leukemia. Oncogene 33:403-410.
Carter et al. (1992) Humanization of an anti-p185HER2 antibody for human cancer therapy. Proc Natl Acad Sci USA 89:4285-4289.
Chothia et al. (1989) Conformations of immunoglobulin hypervariable regions. Nature 342:877-883.
Clackson et al. (1991) Making antibody fragments using phage display libraries. Nature 352: 624-628.
Clark et al. (1989) The improved lytic function and in vivo efficacy of monovalent monoclonal CD3 antibodies. Eur J Immunol 19:381-388.
Clothia et al. (1985) Domain association in immunoglobulin molecules. The packing of variable domains. J Mol Biot 186:651-666.
Cobbold et al. (2013) MHC class I-associated phosphopeptides are the targets of memory-like immunity in leukemia. Sci Trans Med 5(203): 1-10.
Cole et al. (1985) Identification of an additional class of C3-binding membrane proteins of human peripheral blood leukocytes and cell lines. Proc Natl Acad Sci USA 82:859.
Cote et al. (1983) Generation of human monoclonal antibodies reactive with cellular antigens. Proc Natl Acad Sci USA 80:2026.
Coule et al. (2014) Tumor antigens recognized by T-lymphocytes: at the core of cancer immunotherapy. Nat Rev Cancer 14:135-146.
Coulie et al. (1995) A mutated intron sequence codes for an antigenic peptide recognized by cytolytic T lymphocytes on a human melanoma. Proc Natl Acad Sci USA 92:7976-7980.
Crooks et al. (2004) WebLogo: A sequence logo generator, Genome Res 14:1188-1190.
de Queiroz et al. (2014) O-GlcNAcylation: the sweet side of the cancer. Front Oncol 4(132):1-10.
Dephoure et al. (2008) A quantitative atlas of mitotic phosphorylation. Proc Natl Acad Sci USA 105:10762-10767.
Devereux et al. (1984) A comprehensive set of sequence analysis programs for the VAX. Nucl Acids Res 12:387-395.
Dudley et al. (2003) Generation of tumor-infiltrating lymphocyte cultures for use in adoptive transfer therapy for melanoma patients. J Immunother 26(4):332-342.
Earley et al. (2013) Front-end electron transfer dissociation: A new ionization source. Anal Chem 85 (17):8385-8390.
Fishwild et al. (1996) High-avidity human IgG kappa monoclonal antibodies from a novel strain of minilocus transgenic mice. Nature Biotechnol 14:845.
Gattinoni et al. (2006) Adoptive immunotherapy for cancer: building on success. Nat Rev Immunol 6: 383-93.
Giancotti (2014) Deregulation of cell signaling in cancer, FEBS Lett 588:2558-2570.
Githero et al. (1999) Crystal structures of two H-2Db/glycopeptides complexes suggest a molecular basis for CTL cross-reactivity. Immunity 19(1):63-74.
Great Britain Patent Publication GB 2249310A.
Gross & Mienhofer (eds.) (1981) The Peptides, Vol. 3, Academic Press, New York, N.Y., United States of America, pp. 3-88.
Gubin et al. (2015) Tumor neoantigens: building a framework for personalized cancer immunotherapy. J Clin Iinvest 125:3413.3421.
Hamann et al. (1997) Phenotypic and functional separation of memory and effector human CD8⁺ T cells. J Exp Med 186:1407-1418.
Hanahan & Weinberg (2011) Hallmarks of cancer: the next generation. Cell 144:646-674.
Harlow & Lane (1988) Antibodies: A Laboratory Manual, Cold Spring Harbor Laboratory Press, Cold Spring Harbor, N.Y., United States of America.
Hart (2014) Minireview series on the thirtieth anniversary of research on 0-GlcNAcylation of nuclear and cytoplasmic proteins: nutrient regulation of cellular metabolism and physiology by O-GlcNAcylation. J Biol Chem 289(50): 34422-34423.
Hart et al. (2011) Cross talk between O-GlcNAcylation and phosphorylation: roles in signaling, transcription, and chronic disease. Annu Rev Biochem 80:825-858.
Haurum et al. (1994) Recognition of carbohydrate by major histocompatibility complex class I-restricted, glycopeptide-specific cytotoxic T lymphocytes. J Exp Med 180(2):739-744.
Haurum et al. (1995) Peptide anchor residue glycosylation: effect on class I major histocompatibility complex binding and cytotoxic T lymphocyte recognition.
Eur J Immunol 25(12):3270-3276.
Haurum et al. (1999) Presentation of cytosolic glycosylated peptides by human class I to major histocompatibility complex molecules in vivo. J Exp Med 190(1):145-150.
Hoogenboom et al. (1991) Multi-subunit proteins on the surface of filamentous phage: methodologies for displaying antibody (Fab) heavy and light chains. Nucleic Acids Res 19:4133.
Jones et al. (1986) Replacing the complementarity-determining regions in a human antibody with those from a mouse. Nature 321:522-525.
Kabat et al. (1991) “Sequences of Proteins of Immunological Interest, Fifth Edition”, NIH Publication No. 91-3242, National Institute of Health, Bethesda, Md., United States of America.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kastrup et al. (2000) Lectin purified human class I MHC-derived peptides: evidence for presentation of glycopeptides in vivo. Tissue Antigens 56(2):129-135.
Kohler & Milstein (1975) Continuous cultures of fused cells secreting antibody of predefined specificity. Nature 256:495-475.
Kozbor et al. (1983) A comparative analysis of the phenotypic characteristics of available fusion partners for the construction of human hybridomas. Hybridoma 2:7-16.
Lawrence et al. (2013) Mutational heterogeneity in cancer and the search for new cancer-associated genes. Nature 499:214-218.
LeFranc & LeFranc (2001) The T cell Receptor FactsBook, Academic Press, San Diego, Calif., United States of America, ISBN 0-12-441352-8.
Linnemann et al. (2015) High-throughput epitope discovery reveals frequent recognition of neo-antigens by CD4+ T cells in human melanoma. Nature Med 21:81-85.
Lonberg & Huszar (1995) Human antibodies from transgenic mice. Int Rev Immunol 13:65.
Lonberg et al. (1994) Antigen-specific human antibodies from mice comprising four distinct genetic modifications. Nature 368:856.
Mackensen et al. (2000) Phase I study in melanoma patients of a vaccine with peptide-pulsed dendritic cells generated in vitro from CD34(+) hematopoietic progenitor cells. Int Cancer 86:385-392.
Marks et al. (1991) By-passing immunization. Human antibodies from V-gene libraries displayed on phage. J Mol Biol 222:581-597.
Marks et al. (1992) Molecular evolution of proteins on filamentous phage. Mimicking the strategy of the immune system. J Biot Chem 267:16007.
McGranahan et al. (2016) Clonal neoantigens elicit T cell immunoreactivity and sensitivity to immune checkpoint blockade. Science 351:1463-1469.
Melief (2009) Immunotherapy of cancer by vaccination with long synthetic peptides. J Med Sci 2:43-45.
Mellquist et al. (1998) The amino acid following an asn-X-Ser/Thr sequon is an important determinant of N-linked core glycosylation efficiency. Biochemistry 37(19):6833-6837.
Mihara et al. (2001) Humanized antibody to human interleukin-6 receptor inhibits the development of collagen arthritis in cynomolgus monkeys. Clin Immunol 98:319.
Mohammed et al. (2008) Phosphorylation-dependent interaction between antigenic peptides and MHC class I: a molecular basis for the presentation of transformed self. Nat Immunol 9:1236-1243.
Molina et al. (2007) Global proteomic profiling of phosphopeptides using electron transfer dissociation tandem mass spectrometry. Proc Natl Acad Sci USA 104: 2199-2204.
Motzer et al. (2015) Nivolumab versus Everolimus in Advanced Renal-Cell Carcinoma. N Eng J Med 373:1803-1813.
Neuberger (1996) Generating high-avidity human Mabs in mice. Nat Biotechnol 14:826.
Novotny & Haber (1985) Structural invariants of antigen binding: comparison of immunoglobulin VL-VH and VL-VL domain dimers. Proc Natl Acad Sci USA 82:4592-4596.
Pardoll (2012) The blockade of immune checkpoints in cancer immunotherapy. Nature Reviews Cancer 12:252-264.
PCT International Patent Application Publication Nos. WO 1994/02602; WO 1996/33735; WO 1996/34096; WO 1999/047929; WO 2004/106380.
PCT International Patent Application Serial No. PCT/US2013/058477.
Richards et al. (1999) Phase I evaluation of humanized OKT3: toxicity and immunomodulatory effects of hOKT3gamma4. Cancer Res 59:2096.
Riechmann et al. (1988) Reshaping human antibodies for therapy. Nature 332:323-327.
Rizvi et al. (2015) Cancer immunology. Mutational landscape determines sensitivity to PD-1 blockade in non-small cell lung cancer. Science 348:124-128.
Rooney et al. (2015) Molecular and genetic properties of tumors associated with local immune cytolytic activity. Cell 160:48-61.
Rowe et al. (eds.) (2006) Handbook of Pharmaceutical Excipients, 5th Ed., Pharmaceutical Press, London, United Kingdom.
Rughetti et al. (2013) Microvesicle cargo of tumor-associated MUC1 to dendritic cells allows cross-presentation and specific carbohydrate processing. Cancer Immunol Res 2(2):177-186.
Sandborn et al. (2001) An engineered human antibody to TNF (CDP571) for active Crohn's disease: a randomized double-blind placebo-controlled trial. Gastroenterology, 120:1330-1338.
Schneider & Stephens (1990) Sequence Logos: A new way to display consensus sequences. Nucl Acids Res 18:6097-6100.
Shinkura et al. (1998) In vivo blocking effects of a humanized antibody to human interleukin-6 receptor on interleukin-6 function in primates. Anticancer Res 18:1217.
Slawson & Hart (2011) O-GlcNAc signaling: implications for cancer biology. Nat Rev Cancer 11:678-684.
Slawson et al. (2005) Perturbations in O-linked β-N-acetylglucosamine protein modification cause severe defects in mitotic progression and cytokinesis. J Biol Chem 280:32944-32956.
Slawson et al. (2008) A mitotic GlcN Acylation/phosphorylation signaling complex alters the posttranslational state of the cytoskeletal proteim vimentin. Mol Biol Cell 19:4130-4140.
Strickley (2004) Solubilizing excipients in oral and injectable formulations. Pharm Res 21:201-230.
Suzuki et al. (2016) The cytoplasmic peptide:N-glycanase (NGLY1)—Structure, expression and cellular functions. Gene 577:1-7.
Syka et al. (2004) Peptide and protein sequence analysis by electron transfer dissociation mass spectrometry. Proc Natl Acad Sci USA 101(26): 9528-9533.
Udeshi et al. (2008) Methods for analyzing peptides and proteins on a chromatographic timescale by electron-transfer dissociation mass spectrometry. Nat Protoc 3(11):1709-1717.
U.S. Patent Application Publication Nos. 2002/0119149; 2004/0202657; 2009/0117102; 2009/0226474.
U.S. Pat. Nos. 4,361,539; 4,714,681; 4,816,567; 5,225,539; 5,545,806; 5,545,807; 5,569,825; 5,625,126; 5,661,016; 5,633,425; 5,712,120; 5,861,155; 5,916,771; 5,939,598; 5,968,509; 6,054,927; 6,180,370; 6,706,265; 6,750,325; 8,912,149; 9,249,187.
Van Wauve (1980) OKT3: a monoclonal anti-human T lymphocyte antibody with potent mitogenic properties. J Immunol 124:2708-2718.
Verhoeyen et al. (1988) Reshaping human antibodies: grafting an antilysozyme activity. Science 239:1534-1536.
Wang et al. (2007) Dynamic interplay between O-linked N-acetylglucosaminylation and glycogen synthase kinase-3-dependent phosphorylation. Mol Cell Proteomics 6:1365-1379.
Wang et al. (2010a) Enrichment and site-mapping of O-linked N-acetylglucosamine by a combination of chemical/enzymatic tagging, photochemical cleavage, and electron transfer dissociation (ETD) mass spectrometry. Mol Cell Proteomics 9(1):153-160.
Wang et al. (2010b) Extensive crosstalk between GlcNAc-ylation and phosphorylation regulates cytokinesis. Sci Signal 3(104):ra2.
Wells et al. (2004)O-GlcNAc transferase is in a functional complex with protein phosphatase 1 catalytic subunits. J Biol Chem 279(37):38466-38470.
Wolfert & Boons (2013) Adaptive immune activation: glycosylation does matter. Nature Chem Biol 9:776-784.
Wong (1990) The mechanism of anti-CD3 monoclonal antibodies. Mediation of cytolysis by inter-T cell bridging. Transplantation 50:683-689.
Yee et al. (2002) Adoptive T cell therapy using antigen-specific CD8+ T cell clones for the treatment of patients with metastatic melanoma: in vivo persistence, migration, and antitumor effect of transferred T cells. Proc Natl Acad Sci USA 99:16168-16173.
Yenari et al. (1998) Hu23F2G, an antibody recognizing the leukocyte CD11/CD18 integrin, reduces injury in a rabbit model of transient focal cerebral ischemia. Exp Neurol 153:223.
Zhao et al. (2011) Combining high-energy C-trap dissociation and electron transfer dissociation for protein O-GlcNAc modification site assignment. J Proteome Res 10: 4088-4104.

Headings are included herein for reference and to aid in locating certain sections. These headings are not intended to limit the scope of the concepts described therein under, and these concepts can have applicability in other sections throughout the entire specification.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A composition comprising at least or about 1, 2, 3, 4, 5, 6, 7, 8, 9, 10, or more synthetic target peptides, wherein each synthetic target peptide:
(i) is about or at least 8, 9, 10, 11, 12, 13, 14, or 15 amino acids long; and
(ii) comprises an amino acid sequence as set forth in any of SEQ ID NOs: 1-45,
and further wherein said composition optionally stimulates a T cell-mediated immune response to at least one of the synthetic target peptides.

2. The composition of claim 1, wherein at least one of the synthetic target peptides comprises a substitution of a serine residue with a homo-serine residue.

3. The composition of claim 1, wherein at least one of the synthetic target peptides is an O-GlcNAcylated peptide or a mimetic thereof.

4. The composition of claim 1, wherein at least one of the synthetic target peptides is a methylated peptide or a mimetic thereof.

5. The composition of claim 1, wherein at least one of the synthetic target peptides is a peptide that is both O-GlcNAcylated and methylated.

6. The composition of claim 5, wherein at least one of the synthetic target peptides comprises an O-GlcNAcylated serine and a methylated arginine.

7. The composition of claim 1, wherein the composition is immunologically suitable for administration to a leukemia patient.

8. The composition of claim 1, wherein the composition comprises at least 5 different target peptides.

9. The composition of claim 1, wherein the composition comprises at least 10 different target peptides.

10. The composition of claim 1, wherein the composition comprises at least 15 different target peptides.

11. The composition of claim 1, wherein at least one of the synthetic target peptides is capable of binding to an MEW class I molecule of the HLA-A*0201 allele, a B*0702 allele, or a B*35 allele.

12. The composition of claim 1, wherein the composition is capable of increasing the 5-year survival rate of leukemia patients treated with the composition by at least 20 percent relative to average 5-year survival rates that could have been expected without treatment with the composition.

13. The composition of claim 1, wherein the composition is capable of increasing the survival rate of leukemia patients treated with the composition by at least 20 percent relative to a survival rate that could have been expected without treatment with the composition.

14. The composition of claim 1, wherein the composition is capable of increasing the treatment response rate of leukemia patients treated with the composition by at least 20 percent relative to a treatment response rate that could have been expected without treatment with the composition.

15. The composition of claim 1, wherein the composition is capable of increasing the overall median survival of patients of leukemia patients treated with the composition by at least two months relative to an overall median survival that could have been expected without treatment with the composition.

16. The composition of claim 1, further comprising at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, and TPS.

17. The composition of claim 1, wherein the composition further comprises an adjuvant.

18. The composition of claim 17, wherein the adjuvant is selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof.

19. An in vitro population of antigen presenting cells, optionally dendritic cells, comprising the composition of any one of claims 1-18 or a composition comprising at least one target peptide comprising an amino acid sequence as set forth in any of SEQ ID NOs: 1-45.

20. An in vitro population of CD8⁺ T cells capable of being activated upon being brought into contact with a population of antigen presenting cells, optionally dendritic cells, wherein the antigen presenting cells, optionally comprise the composition of any one of claims 1-18 or a composition comprising at least one target peptide comprising an amino acid sequence as set forth in any of SEQ ID NOs: 1-45.

21. An in vitro population of CD8+ T cells capable of being activated upon being brought into contact with a complex of an MHC class I molecule and a peptide comprising an amino acid sequence as set forth in any of SEQ ID NOs: 1-45.

22. An antibody or antibody-like molecule that specifically binds to a complex of an MHC class I molecule and a peptide comprising an amino acid sequence as set forth in one or more of SEQ ID NOs: 1-45.

23. The antibody or antibody-like molecule of claim 22, wherein the peptide is 0-GlcNAcylated and/or methylated, and wherein the antibody or antibody-like molecule does not substantially cross react with the corresponding non-O-GlcNAcylated and/or non-methylated peptide.

24. The antibody or antibody-like molecule of claim 22, wherein the antibody or antibody-like molecule is a member of the immunoglobulin superfamily.

25. The antibody or antibody-like molecule of claim 22, wherein the antibody or antibody-like molecule comprises a binding member selected from the group consisting of an Fab, Fab′, F(ab′)₂, Fv, and a single-chain antibody.

26. The antibody or antibody-like molecule of claim 22 conjugated to a therapeutic agent selected from the group consisting of an alkylating agent, an antimetabolite, a mitotic inhibitor, a taxoid, a vinca alkaloid, and an antibiotic.

27. The antibody or antibody-like molecule of claim 22, wherein the antibody or antibody-like molecule is a T cell receptor.

28. The antibody or antibody-like molecule of claim 27, wherein the antibody or antibody-like molecule is conjugated to a CD3 agonist.

29. An isolated polynucleotide encoding the antibody or antibody-like molecule of claim 22 or a chain thereof, or the T cell receptor of claim 27 or a chain thereof.

30. A vector comprising the polynucleotide of claim 29.

31. A recombinant host cell comprising the polynucleotide of claim 29 or the to vector of claim 30.

32. The host cell of claim 31, wherein the host cell is a T cell comprising the polynucleotide of claim 29, wherein the polynucleotide encodes the T cell receptor of claim 27 or a chain thereof.

33. A method for treating and/or preventing cancer comprising administering to a subject in need thereof a therapeutically effective dose of the composition of any one of claims 1-18 or a composition comprising at least one target peptide comprising an amino acid sequence as set forth in any of SEQ ID NOs: 1-45 in combination with a pharmaceutically acceptable carrier.

34. A method of treating and/or preventing leukemia comprising administering to a subject in need thereof a therapeutically effective dose of the composition of any one of claims 1-18 or a composition comprising at least one target peptide comprising an amino acid sequence as set forth in any of SEQ ID NOs: 1-45 in combination with a pharmaceutically acceptable carrier.

35. A method for treating and/or preventing cancer comprising administering to a subject in need thereof a therapeutically effective dose of the in vitro population of dendritic cells of claim 19 in combination with a pharmaceutically acceptable carrier.

36. A method for treating and/or preventing cancer comprising administering to a subject in need thereof a therapeutically effective dose of the in vitro population of CD8⁺ T cells of claim 20 in combination with a pharmaceutically acceptable carrier.

37. A method for treating and/or preventing cancer comprising administering to a subject in need thereof a therapeutically effective dose of the antibody or antibody-like molecule of claim 22, the T cell receptor of claim 27, or the host cell of claim 31 or 32 in combination with a pharmaceutically acceptable carrier.

38. A method for making a cancer vaccine comprising combining the composition of any one of claims 1-18 with an adjuvant selected from the group consisting of montanide ISA-51, QS-21, a tetanus helper peptide, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanin (KLH), complete Freunds adjuvant, in complete Freunds adjuvant, a mineral gel, aluminum hydroxide (Alum), lysolecithin, a pluronic polyol, a polyanion, an adjuvant peptide, an oil emulsion, dinitrophenol, and diphtheria toxin (DT), or any combination thereof and a pharmaceutically acceptable carrier; and placing the composition, adjuvant, and pharmaceutical carrier into a container, optionally into a syringe.

39. A method for screening target peptides for inclusion in the composition of any one of claims 1-18 or for use in a method of using the composition of any one of claims 1-18, comprising:
(a) administering the target peptide to a human;
(b) determining whether the target peptide is capable of inducing a target peptide-specific memory T cell response in the human; and
(c) selecting the target peptide for inclusion in the composition or for use in the method of using the composition if the target peptide elicits a memory T cell response in the human.

40. A method for determining a prognosis of a leukemia patient, the method comprising:
(a) administering to the patient a target peptide comprising an amino acid sequence as set forth in any of SEQ ID NOs: 1-45, wherein the target peptide is associated with the patient's leukemia;
(b) determining whether the target peptide is capable of inducing a target peptide-specific memory T cell response in the patient; and
(c) determining that the patient has a better prognosis if the patient mounts a memory T cell response to the target peptide than if the patient did not mount a memory T cell response to the target peptide.

41. A kit comprising at least one target peptide composition comprising at least one target peptide comprising an amino acid sequence as set forth in any of SEQ ID NOs: 1-45 and a cytokine and/or an adjuvant.

42. The kit of claim 41, comprising at least 2, 3, 4, or 5 target peptide compositions.

43. The kit of claim 41, wherein the at least one target peptide composition is one of the compositions of claims 1-18.

44. The kit of claim 41, wherein the cytokine is selected from the group consisting of a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-alpha, interferon-beta, and/or interferon-gamma; and a colony stimulating factor (CSF), optionally macrophage-CSF (M-CSF), granulocyte-macrophage-CSF (GM-CSF), and/or granulocyte-CSF (G-CSF).

45. The kit of claim 41, wherein the adjuvant is selected from the group consisting of montanide ISA-51, QS-21, a tetanus helper peptide, GM-CSF, cyclophosphamide, bacillus Calmette-Guerin (BCG), corynbacterium parvum, levamisole, azimezone, isoprinisone, dinitrochlorobenezene (DNCB), a keyhole limpet hemocyanin (KLH), complete Freund's adjuvant, incomplete Freund's adjuvant, a mineral gel, aluminum hydroxide, lysolecithin, a pluronic polyol, a polyanion, an adjuvant peptide, an oil emulsion, dinitrophenol, and diphtheria toxin (DT).

46. The kit of claim 41, wherein the cytokine is selected from the group consisting of a nerve growth factor, optionally nerve growth factor (NGF) beta; a platelet-growth factor; a transforming growth factor (TGF), optionally TGF-alpha and/or TGF-beta; insulin-like growth factor-I; insulin-like growth factor-II; erythropoietin (EPO); an osteoinductive factor; an interferon, optionally interferon-α, interferon-β, and/or interferon-γ; a colony stimulating factor (CSF), optionally macrophage-CSF (M-CSF), granulocyte-macrophage-CSF (GM-CSF), and/or granulocyte-CSF (G-CSF); an interleukin (IL), optionally IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, and/or IL-18; LIF; EPO; kit-ligand; fms-related tyrosine kinase 3 (FLT-3; also called CD135); angiostatin; thrombospondin; endostatin; tumor necrosis factor; and lymphotoxin (LT).

47. The kit of claim 41, further comprising at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS.

48. The kit of claim 41, wherein the at least one target peptide comprises an amino acid sequence as set forth in any of SEQ ID NOs: 1-45.

49. The composition of claim 1, comprising a peptide capable of binding to an MEW class I molecule of the HLA A*0201 allele, the B*0702 allele, or the B*35 allele.

50. A composition comprising at least one synthetic target peptide, wherein each synthetic target peptide:
(i) is between 8 and 50 amino acids long, and
(ii) comprises an amino acid sequence selected from the group consisting of SEQ ID NOs: 1-45.

51. The composition of claim 50, wherein at least one serine residue in at least one of the synthetic target peptides is replaced with a homo-serine residue.

52. The composition of claim 50, wherein the composition comprises at least 5, 10, 15, or 20 different peptides.

53. The composition of claim 50, further comprising at least one peptide derived from MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, and TPS.

54. The composition of claim 50, further comprising an agent selected from the group consisting of anti-CTLA-4 antibody, vermurafenib, ipilimumab, dacarbazine, IL-2, temozolomide, imatinib, gefitinib, erlotinib, sunitinib, tyrphostins, and telatinib.

55. The composition of claim 50, further comprising darcarbazine, carmustine, and tamoxifen.

56. The composition of claim 50, wherein the composition further comprises an adjuvant.

57. The composition of claim 50, wherein at least one of the synthetic target peptides is O-GlcNAcylated.

58. The composition of claim 50, wherein at least one of the synthetic target peptides comprises:
the amino acid sequence set forth in any of SEQ ID NOs: 9, 13, 14, and 41, and further wherein the amino acid at the third position in the sequence is O-GlcNAcylated; and/or
the amino acid sequence set forth in any of SEQ ID NOs: 1, 2, 4-20, 24-28, 31, 33, and 35-45, and further wherein the amino acid at the fourth position in the sequence is O-GlcNAcylated; and/or
the amino acid sequence set forth in any of SEQ ID NOs: 2, 3, 9, 12, 14, 19, 21, 23, 28-32, 34, and 41, and further wherein the amino acid at the fifth position in the sequence is O-GlcNAcylated; and/or
the amino acid sequence set forth in any of SEQ ID NOs: 2, 6, 23, and 31, and further wherein the amino acid at the sixth position in the sequence is O-GlcNAcylated; and/or
the amino acid sequence set forth in any of SEQ ID NOs: 22 and 25, and further wherein the amino acid at the seventh position in the sequence is O-GlcNAcylated.

59. The composition of claim 50, wherein at least one of the synthetic target peptides comprises the amino acid sequence set forth in SEQ ID NO: 2, and further wherein the amino acid at the fourth, fifth or sixth position, or at both the fourth and fifth positions, or at both the fifth and sixth positions in the sequence is O-GlcNAcylated.

60. The composition of claim 59, wherein:
the amino acid at the fourth, fifth or sixth position comprises a single GlcNAc moiety or a single hexose-GlcNAc moiety; or
the amino acids at the fourth and fifth positions both comprise GlcNAc moieties or hexose-GlcNAc moieties; or
the amino acids at the fifth and sixth positions both comprise GlcNAc moieties or hexose-GlcNAc moieties; or
the amino acids at the fifth position comprises a hexose-GlcNAc moiety and the amino acid at the sixth position comprises a GlcNAc moiety.

61. The composition of claim 50, wherein at least one of the synthetic target peptides comprises the amino acid sequence set forth in SEQ ID NO: 3, and further wherein the amino acid at the fifth position comprises an N-linked hexose-GlcNAc moiety.

62. The composition of claim 50, wherein at least one of the synthetic target peptides comprises the amino acid sequence set forth in SEQ ID NO: 12, and further wherein the amino acid at the fourth position comprises a GlcNAc moiety or the amino acids at the fourth and fifth positions both comprise a GlcNAc moiety.

63. The composition of claim 50, wherein at least one of the synthetic target peptides comprises the amino acid sequence set forth in SEQ ID NO: 18, and further wherein the amino acid at the fourth position comprises a GlcNAc moiety or a hexose-GlcNAc moiety.

64. The composition of claim 50, wherein at least one of the synthetic target peptides comprises the amino acid sequence set forth in SEQ ID NO: 19, and further wherein the amino acid at the fourth position comprises a GlcNAc moiety, a hexose-GlcNAc moiety, or an acetyl-GlcNAc moiety; or the amino acids at the fourth and fifth positions both comprise a GlcNAc moiety.

65. The composition of claim 50, wherein at least one of the synthetic target peptides comprises the amino acid sequence set forth in SEQ ID NO: 20, and further wherein the amino acid at the fourth position comprises a GlcNAc moiety or a hexose-GlcNAc moiety.

66. The composition of claim 50, wherein at least one of the synthetic target peptides comprises the amino acid sequence set forth in SEQ ID NO: 21, and further wherein the amino acid at the fifth position comprises a hexose-GlcNAc moiety.

67. The composition of claim 50, wherein at least one of the synthetic target peptides comprises the amino acid sequence set forth in SEQ ID NO: 22, and further wherein the amino acid at the seventh position comprises an N-linked hexose-GlcNAc moiety.

68. The composition of claim 50, wherein at least one of the synthetic target peptides comprises the amino acid sequence set forth in SEQ ID NO: 25, and further wherein the amino acid at the seventh position comprises a hexose-GlcNAc moiety or the amino acid at the fourth position comprises an asymmetric di-methyl moiety and the amino acid at the seventh position comprises a hexose-GlcNAc moiety.

69. The composition of claim 50, wherein at least one of the synthetic target peptides comprises the amino acid sequence set forth in SEQ ID NO: 28, and further wherein the amino acid at the fourth position or the amino acid at the fifth position comprises an O-linked hexose-GlcNAc moiety.

70. The composition of claim 50, wherein at least one of the synthetic target peptides comprises the amino acid sequence set forth in SEQ ID NO: 29, and further wherein the amino acid at the fifth position comprises a GlcNAc moiety, the amino acid at the first position comprises a mono-methyl moiety and the amino acid at the fifth position comprises a GlcNAc moiety, the amino acid at the first position comprises an asymmetric di-methyl moiety and the amino acid at the fifth position comprises a GlcNAc moiety, or the amino acid at the first position comprises an asymmetric di-methyl moiety and the amino acid at the fifth position comprises an acetyl-GlcNAc moiety.

71. The composition of claim 50, wherein at least one of the synthetic target peptides comprises the amino acid sequence set forth in SEQ ID NO: 30, and further wherein the amino acid at the fifth position comprises an O-linked hexose-GlcNAc moiety.

72. The composition of claim 50, wherein the composition has the ability to stimulate a T cell mediated immune response to at least one of the synthetic target peptides.

73. The composition of claim 56, wherein the adjuvant is selected from the group consisting of montanide ISA-51, QS-21, tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), incomplete Freunds adjuvant, complete Freunds adjuvant, mineral gels, aluminum hydroxide (Alum), lysolecithin, pluronic polyols, polyanions, peptides, oil emulsions, dinitrophenol, and diphtheria toxin (DT).

74. A composition comprising a synthetic target peptide and an adjuvant, wherein the synthetic target peptide is between 8 and 50 amino acids long and comprises the amino acid sequence set forth in any of SEQ ID NOs: 1-45.

75. The composition of any one of claims 61-71, wherein the composition further comprises an adjuvant.

76. The composition of claim 74, wherein the adjuvant is QS-21.

77. The composition of claim 75, wherein the adjuvant is QS-21.

78. The composition of any one of claims 1-18, 50-74, 76, and 77, wherein the composition further comprises a pharmaceutically acceptable carrier, optionally a pharmaceutically acceptable carrier that is pharmaceutically acceptable for use in a human.

79. The composition of claim 75, wherein the composition further comprises a pharmaceutically acceptable carrier, optionally a pharmaceutically acceptable carrier that is pharmaceutically acceptable for use in a human.

80. The composition of any one of claims 50-79, wherein the peptide is capable of binding to an MHC class I molecule of the HLA A*0201 allele, the B*0702 allele, or the B*35 allele.
